(19) United States
(12) Patent Application Publication
(10) Pub. No.: US 2021/0403885 A1
(43) Pub. Date: Dec. 30, 2021

(54) CHIMERIC ADAPTOR AND KINASE SIGNALING PROTEINS AND THEIR USE IN IMMUNOTHERAPY

(71) Applicant: The United States of America, as represented by the Secretary, Dept. of Health and Human Services, Bethesda, MD (US)

(72) Inventors: Lakshmi Balagopalan, Bethesda, MD (US); Chang Kyun Yi, Germantown, MD (US); Katherine Marie McIntire, Sandy, UT (US); Lawrence Elliot Samelson, Bethesda, MD (US)

(73) Assignee: The United States of America, as represented by the Secretary, Dept. of Health and Human Services, Bethesda, MD (US)

(21) Appl. No.: 17/475,810

(22) Filed: Sep. 15, 2021

Related U.S. Application Data
(63) Continuation-in-part of application No. PCT/US2020/022752, filed on Mar. 13, 2020.
(60) Provisional application No. 62/819,386, filed on Mar. 15, 2019.

(51) Int. Cl. C12N 9/12; C07K 14/705; C07K 14/535; C07K 16/28; A61K 35/17; A61P 35/02; A61P 35/00

(52) U.S. Cl. CPC C12N 9/12; A61K 2039/5156; C07K 14/70578; C07K 14/70521; C07K 14/70517; C07K 14/535; C07K 16/2803; A61K 35/17; C07K 16/2812; A61P 35/02; A61P 35/00; C07K 2319/03; C07K 2319/02; C07K 2317/622; C07K 2319/30; C07K 2319/33; C07K 2317/76; C12Y 207/10002

(57) ABSTRACT

Chimeric polypeptides including (a) an extracellular targeting domain; (b) a transmembrane domain; (c) an intracellular linker for activation of T cells (LAT) domain or SLP-76 domain; and (d) an intracellular ZAP70 domain, wherein (a)-(d) are in N-terminal to C-terminal order are provided. Chimeric polypeptides including (a) an extracellular targeting domain; (b) a transmembrane domain; and (c) a ZAP70 domain, wherein (a)-(c) are in N-terminal to C-terminal order are also provided. In some embodiments, the chimeric polypeptide further includes a hinge domain, a signal sequence domain, and/or an intracellular signaling domain. Nucleic acid molecules encoding the chimeric polypeptides and expression vectors including the nucleic acids are also provided. Isolated cells (such as T cells or natural killer cells) expressing the chimeric polypeptides and methods of treating a subject with cancer with the isolated cells are provided.

### CROSS REFERENCE TO RELATED APPLICATIONS

This application is a continuation-in-part of International Application No. PCT/US2020/022752, filed Mar. 13, 2020, which claims the benefit of U.S. Provisional Application No. 62/819,386, filed Mar. 15, 2019, each of which is incorporated by reference herein in its entirety.

### ACKNOWLEDGMENT OF GOVERNMENT SUPPORT

This invention was made with Government support under project number Z01 BC 010304 by the National Institutes of Health, National Cancer Institute. The Government has certain rights in the invention.

### FIELD

This disclosure relates to chimeric adaptor proteins, which in some examples include a Linker for Activation of T cells (LAT) domain and/or a ZAP70 domain, and their use in immunotherapy.

### BACKGROUND

Chimeric antigen receptors (CARs) are molecules composed of an antibody fragment specific for a tumor antigen, fused to a transmembrane domain and a T-cell-signaling moiety. When expressed on the surface of T cells, CARs mediate binding to a target and activate the T cells, ultimately inducing target cell lysis. CARs are emerging as a promising approach to treat hematological malignancies, including non-Hodgkin lymphoma, B-cell acute lymphoblastic leukemia, and multiple myeloma and use of CARs for treating solid tumors is also being tested. However, T cell exhaustion remains a challenge in CAR-T therapies.

### SUMMARY

There remains a need for effective immunotherapies to treat solid tumors as well as hematological malignancies. Disclosed herein are chimeric adaptor proteins (CAPs) that can be used in immunotherapy methods for treating cancer, including both hematological and solid malignancies.

Disclosed are chimeric polypeptides (CAPs) including (a) an extracellular targeting domain; (b) a transmembrane domain; (c) an intracellular Linker for Activation of T cells (LAT) domain; and (d) an intracellular ZAP70 domain, wherein (a)-(d) are in N-terminal to C-terminal order. In some embodiments, the LAT domain is replaced with an SLP-76 domain. In further embodiments, the CAPs further include an intracellular signaling domain (such as a 41BB or CD28 intracellular signaling domain) that is C-terminal to the transmembrane domain and N-terminal to the LAT or SLP-76 domain. In some embodiments, the chimeric polypeptide further includes a hinge domain (such as a CD8 or CD28 hinge domain) that is C-terminal to the extracellular targeting domain and N-terminal to the transmembrane domain. In additional embodiments, the chimeric polypeptide further includes a signal sequence domain (such as a GM-CSF signal sequence) that is N-terminal to the extracellular targeting domain. Linkers (e.g., spacers) may be present between any of the components of the disclosed CAPs, for example, to allow proper folding and/or function of the CAP.

In additional embodiments, the CAPs are chimeric polypeptides including (a) an extracellular targeting domain; (b) a transmembrane domain; and (c) a ZAP70 domain, wherein (a)-(c) are in N-terminal to C-terminal order. In some embodiments, the CAPs further include a 41BB or CD28 intracellular signaling domain that is C-terminal to the transmembrane domain and N-terminal to the ZAP70 domain. In some embodiments, the CAPs further include a hinge domain that is C-terminal to the extracellular targeting domain and N-terminal to the transmembrane domain. In additional embodiments, the CAPs further include a signal sequence domain that is N-terminal to the extracellular signaling domain. Linkers (e.g., spacers) may be present between any of the components of the disclosed CAPs, for example, to allow proper folding and/or function of the CAP.

In some embodiments, the ZAP70 domain is a full length ZAP70 kinase, a ZAP70 kinase domain, or a ZAP70 interdomain B and a ZAP70 kinase domain. In some examples, the full length ZAP70 includes the amino acid sequence of amino acids 377-995 of SEQ ID NO: 55, the ZAP70 kinase domain includes the amino acid sequence of amino acids 527-789 of SEQ ID NO: 5, or the ZAP70 interdomain B includes the amino acid sequence of amino acids 522-604 of SEQ ID NO: 9 or amino acids 522-604 of SEQ ID NO: 11. The ZAP70 domain may include an amino acid substitution at an amino acid position corresponding to M503 of SEQ ID NO: 27, C494 of SEQ ID NO: 27, K593 of SEQ ID NO: 55, Y668 of SEQ ID NO: 55, Y691 of SEQ ID NO: 55, Y695 of SEQ ID NO: 55, or a combination of two or more thereof.

In some examples, the extracellular targeting domain is an antibody, antigen binding domain, or scFv that binds to a target protein of interest, such as a tumor associated antigen (e.g., an antigen expressed by cancer cells). In non-limiting examples, the extracellular targeting domain binds to CD19, CD22, or BCMA. In some examples, the transmembrane domain is a CD8 transmembrane domain, a CD28 transmembrane domain, or a LAT transmembrane domain. In additional examples, the LAT domain is a full-length LAT polypeptide, or is amino acids 34-233 of LAT.

In some embodiments, the CAPs disclosed herein include the amino acid sequence of any one of SEQ ID NOs: 5, 7, 9, 16, 17, 19, 21, 23, 25, 27, 29, 31, 33, 35, 37, 39, 41, 43, 45, 47, 49, 51, 53, 55, 57, 59, 61, 63, 65, 70, 72, 74, 76, 78, 80, 82, 84, 86, 88, 90, 92, 94, 96, 98, and 100.

Also disclosed are nucleic acid molecules encoding the CAPs provided herein. In some embodiments, the CAPs are encoded by the nucleic acid sequence of any one of SEQ ID NOs: 6, 8, 10, 15, 18, 20, 22, 24, 26, 28, 30, 32, 34, 36, 38, 40, 42, 44, 46, 48, 50, 52, 54, 56, 58, 60, 62, 64, 66, 71, 73, 75, 77, 79. 81, 83, 85, 87, 89, 91, 93, 95, 97, 99, and 101. In additional embodiments, the nucleic acid molecule is included in an expression vector (such as a lentiviral or retroviral vector). Isolated cells (such as T cells or natural killer cells) expressing the CAPs are provided, as are compositions including the cells and a pharmaceutically acceptable carrier.

Further provided are methods of treating a subject with cancer (such as a hematological malignancy or solid tumor). Such methods include administering to the subject an isolated cell or composition disclosed herein. In some examples, the cells are T cells expressing a CAP, such as autologous T cells. In other examples, the cells are NK cells expressing a CAP, such as autologous NK cells.

The foregoing and other features of the disclosure will become more apparent from the following detailed description, which proceeds with reference to the accompanying figures.

### BRIEF DESCRIPTION OF THE DRAWINGS

FIGS. 1A-1D are a series of panels showing characterization of CD4 CAPs. FIG. 1A is a schematic diagram of LAT-based constructs including CD4 extracellular domain (ED) and full length (FL) LAT (CD4-LAT) or CD4-ED, FL LAT, and ZAP70 kinase domain (KD) (CD4-CAP). These constructs include a CD4 signal sequence and LAT TM domain. E6.1 Jurkat T cells expressing TCRζ-Emerald were activated on anti-CD3 antibody (HIT3)-coated coverslip (FIG. 1B). E6.1 Jurkat T cells expressing CD4-LAT and GRB2-Emerald (FIG. 1C) or CD4-CAP-GFP (FIG. 1D) were activated on anti-CD4 antibody (OKT4)-coated coverslips.

FIGS. 2A and 2B are panels showing microclusters in E6.1 Jurkat T cells expressing CD4-CAP-GFP activated on anti-CD4 antibody (OKT4)-coated coverslip (FIG. 2A) or anti-CD45 (left) or anti-CD43 (right) coated coverslips (FIG. 2B).

FIGS. 3A and 3B are panels showing microclusters in E6.1 Jurkat T cells expressing TCRζ-Halo and CD4-CAP-GFP (FIG. 3A) or ZAP70-Halo and CD4-CAP-GFP (FIG. 3B) activated on anti-CD4 antibody (OKT4)-coated coverslips.

FIGS. 4A-4C are a series of panels showing formation of microclusters in E6.1 Jurkat T cells expressing CD4-CAP-GFP and PLCγ1-Halo (FIG. 4A), SLP76-Halo (FIG. 4B), or GRB2-Halo (FIG. 4C) activated on anti-CD4 antibody (OKT4)-coated coverslips.

FIGS. 5A and 5B are a schematic diagram of the anti-CD19-scFv-LZ CAP (FIG. 5A) and a composite DIC image of CD19-LZ-GFP and GRB2-Scarlet-expressing E6.1 cells forming a conjugate with a Raji B cell (FIG. 5B).

FIG. 6 is a schematic diagram showing exemplary SLP76-containing CAP constructs.

FIG. 7 is a schematic diagram showing additional exemplary CAP constructs.

FIGS. 8A and 8B are graphs showing cytokine production by the indicated CAP constructs (shown in FIG. 7) in activated primary human T cells. Transduced or untransduced PBMCs were co-cultured with NALM6 or NALM6 CD19 knockout tumor cells or K562 (not expressing CD19) or K562 CD19 (expressing CD19) at a 1:1 ratio. IFNγ (FIG. 8A) or IL2 (FIG. 8B) release were measured by ELISA after 24 hours.

FIG. 9 is a graph showing tumor cell killing by the indicated CAP constructs (as shown in FIG. 7) expressed in primary human T cells. Transduced or untransduced PBMCs were co-cultured with NALM6 or NALM6 CD19 knockout tumor cells stably expressing luciferase at a 5:1 or 1:1 ratio as indicated. After 4 hours, luminescence was detected by adding Steady Glo reagent (Promega). Luminescence from target only wells (max CPS) and target only wells plus 1% Tween-20 (min CPS) was used to determine assay range. Percent specific lysis was calculated as: 1-(sample CPS-min CPS)/(max CPS-min CPS).

FIGS. 10A-10C are schematic diagrams showing additional CAP constructs. FIG. 10A shows constructs including only the indicated tyrosines in the LAT domain. FIG. 10B shows constructs with the indicated mutations in the ZAP70 domain. FIG. 10C shows second generation versions of CAP2 and CAP4 constructs.

FIG. 11 is a graph showing tumor cell killing by the indicated CAP constructs expressed in primary human T cells. Transduced or untransduced PBMCs were co-cultured with NALM6 or NALM6 CD19 knockout tumor cells stably expressing luciferase. After 4 hours, luminescence was detected by adding Steady Glo reagent (Promega). Luminescence from target only wells (max CPS) and target only wells plus 1% Tween-20 (min CPS) was used to determine assay range. Percent specific lysis was calculated as: 1-(sample CPS-min CPS)/(max CPS-min CPS).

FIGS. 12A and 12B are graphs showing cytokine production by the indicated CAP constructs in activated primary human T cells. Transduced or untransduced PBMCs were co-cultured with K562 (not expressing CD19) or K562 CD19 (expressing CD19) at a 1:1 ratio. IL2 (FIG. 12A) or IFNγ (FIG. 12B) release were measured by ELISA after 24 hours.

FIGS. 13A and 13B show CD4 and CD8 populations of primary human T cells expressing the indicated CAR and CAP constructs.

FIGS. 14A and 14B show basal activation states in CAR and CAP expressing T cells in culture.

FIG. 14A shows basal percentage of naïve T cells (T_{N}), central memory cells (T_{CM}) and effector memory cells (T_{EM}) and FIG. 14B shows basal percentage of exhausted T cells (T_{EX}) on day 8 and 15.

FIG. 15 is a schematic diagram of additional CAP4 constructs.

FIG. 16 is a series of panels showing expression of the indicated CAP4 constructs in T cells.

FIG. 17 is a graph showing tumor cell killing by the indicated CAP constructs expressed in primary human T cells. Transduced or untransduced PBMCs were co-cultured with NALM6 or NALM6 CD19 knockout tumor cells stably expressing luciferase. After 4 hours, luminescence was detected by adding Steady Glo reagent (Promega). Luminescence from target only wells (max CPS) and target only wells plus 1% Tween-20 (min CPS) was used to determine assay range. Percent specific lysis was calculated as: 1-(sample CPS-min CPS)/(max CPS-min CPS).

FIGS. 18A and 18B are graphs showing cytokine production by the indicated CAP constructs in activated primary human T cells. Transduced or untransduced PBMCs were co-cultured with K562 (not expressing CD19) or K562 CD19 (expressing CD19) at a 1:1 ratio. IL2 (FIG. 18A) or IFNγ (FIG. 18B) release were measured by ELISA after 24 hours.

FIG. 19 shows in vivo testing of the indicated CAP-T cells in NSG mice challenged with NALM6 tumor cells. Leukemia burden was detected using the Xenogen IVIS Lumina (Caliper Life Sciences).

FIG. 20 is a schematic diagram of additional CAP4 constructs including full-length ZAP70 domains.

FIG. 21 is a schematic diagram of additional CAP4 constructs including full-length ZAP70 domains with the indicated amino acid substitutions in ZAP70 (numbering based on amino acid position in full length ZAP70).

### SEQUENCE LISTING

Any nucleic acid and amino acid sequences listed herein or in the accompanying Sequence Listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

The Sequence Listing is submitted as an ASCII text file in the form of the file named Sequence_Listing.txt, which was created on Sep. 15, 2021, and is 523,620 bytes, which is incorporated by reference herein.

SEQ ID NO: 1 is the amino acid sequence of CD4-LAT. CD4 Signal peptide: amino acids 1-25; CD4 extracellular domain: amino acids 1-391; LAT 2-233 full length: amino acids 393-624.

SEQ ID NO: 2 is the nucleic acid sequence encoding CD4-LAT. CD4 Signal peptide: nucleotides 1-75; CD4 extracellular domain: nucleotides 1-1174; LAT 2-233 full length: nucleotides 1177-1872.

SEQ ID NO: 3 is the amino acid sequence of CD4-CAP. CD4 Signal peptide: amino acids 1-25; CD4 extracellular domain: amino acids 1-391; LAT full length 2-233: amino acids 393-624; linker: amino acids 625-645 (includes aa 318-377 in ZAP-70-end of Interdomain B); ZAP70-KD: amino acids 646-911.

SEQ ID NO: 4 is the nucleic acid sequence encoding CD4-CAP. CD4 Signal peptide: nucleotides 1-75; CD4 extracellular domain: nucleotides 1-1174; LAT full length 2-233: nucleotides 1177-1872; linker: nucleotides 1876-1935 (Linker includes aa 318-377 in ZAP-70-end of Interdomain B); ZAP70-KD: nucleotides 1936-2724.

SEQ ID NO: 5 is the amino acid sequence of anti-CD19-scFV-LZ. CD8 Signal peptide: amino acids 1-21; Myc sequence: amino acids 22-31; Anti-CD19scFV: amino acids 32-242; LAT full length 2-233: amino acids 276-507; linker: amino acids 508-526 (amino acids 509-524 are ZAP70 amino acids 318-333); ZAP70-KD: amino acids 527-789.

SEQ ID NO: 6 is the nucleic acid sequence encoding anti-CD19-scFV-LZ. CD8 Signal peptide: nucleotides 1-63; Myc sequence: nucleotides 64-93; Anti-CD19scFV: nucleotides 94-819; LAT full length 2-233: nucleotides 826-1521; linker: nucleotides 1522-1578 (nucleotides 1525-1572 encode ZAP70 amino acids 318-333); ZAP70-KD: nucleotides 1579-2367.

SEQ ID NO: 7 is the amino acid sequence of LAT-CAP1. GM-CSF Signal peptide: amino acids 1-22; Anti-CD19scFV: amino acids 23-267; LAT full length 2-233: amino acids 271-502 (amino acids 271-273, LAT extracellular/hinge; amino acids 274-295, LAT TM; amino acids 296-502, LAT intracellular domain); linker: amino acids 503-521 (amino acids 504-519 are amino acids 318-333 of ZAP70); ZAP70-KD: amino acids 522-784.

SEQ ID NO: 8 is the nucleic acid sequence encoding LAT-CAP1. GM-CSF Signal peptide: nucleotides 1-66; Anti-CD19scFV: nucleotides 67-801; LAT full length 2-233: nucleotides 811-1506 (nucleotides 811-819, LAT extracellular/hinge; nucleotides 820-885, LAT TM; nucleotides 886-1506, LAT intracellular domain); linker: nucleotides 1507-1563 (nucleotides 1510-1557 encode amino acids 318-333 of ZAP70); ZAP70-KD: nucleotides 1564-2352.

SEQ ID NO: 9 is the amino acid sequence of LAT-CAP2. GM-CSF Signal peptide: amino acids 1-22; Anti-CD19scFV: amino acids 23-267; LAT full length 2-233: amino acids 271-502 (amino acids 271-273, LAT extracellular/hinge; amino acids 274-295, LAT TM; amino acids 296-502, LAT intracellular domain); linker: amino acids 503-521 (amino acids 504-519 are amino acids 318-333 of ZAP70); ZAP70-IB: amino acids 522-604; ZAP70-KD: amino acids 605-867.

SEQ ID NO: 10 is the nucleic acid sequence encoding LAT-CAP2. GM-CSF Signal peptide: nucleotides 1-66; Anti-CD19scFV: nucleotides 67-801; LAT full length 2-233: nucleotides 811-1506 (nucleotides 811-819, LAT extracellular/hinge; nucleotides 820-885, LAT TM; nucleotides 886-1506, LAT intracellular domain); linker: nucleotides 1507-1563 (nucleotides 1510-1557 encode amino acids 318-333 of ZAP70); ZAP70-IB: nucleotides 1564-1812; ZAP70-KD: nucleotides 1813-2601.

SEQ ID NO: 11 is the amino acid sequence of an exemplary CD8 signal sequence.

SEQ ID NO: 12 is the nucleic acid sequence of an exemplary CD8 signal sequence.

SEQ ID NO: 13 is an exemplary nucleic acid sequence of SLP-76

SEQ ID NO: 14 is an exemplary amino acid sequence of SLP-76

SEQ ID NO: 15 is the nucleic acid sequence of an exemplary SLP-76 containing CAP. GM-CSF Signal peptide: nucleotides 1-66; Anti-CD19scFV: nucleotides 67-801; LAT extracellular/hinge LAT TM domain: nucleotides 811-912 (811-819 LAT extracellular/hinge, 820-885 LAT TM, 886-912); full length SLP-76: nucleotides 913-2508, linker: nucleotides 2509-2565 (nucleotides 1510-1557 encode amino acids 318-333 of ZAP70); ZAP70-KD: nucleotides 2566-3354.

SEQ ID NO: 16 is the amino acid sequence of an exemplary SLP-76 containing CAP. GM-CSF Signal peptide: amino acids 1-22; Anti-CD19scFV: amino acids 23-267; LAT extracellular/hinge and LAT TM domain: amino acids 271-301 (aa271-273: LAT extracellular/hinge, aa274-295: LAT TM, aa296-304: amino acids 27-25 near LAT TM); full length SLP76: amino acids 305-836, linker: 837-855 (aa 838-853 is ZAP70 aa 318-333); ZAP70-KD: amino acids 856-1118.

SEQ ID NO: 17 is the amino acid sequence of 28-CAP1. GM-CSF signal peptide aa 1-22; Anti-CD19 scFV aa 23-267; CD28 hinge and TM aa 270-335; LAT 34-233 aa 336-535; linker aa 536-547; ZAP70 kinase domain aa 548-810.

SEQ ID NO: 18 is the nucleic acid sequence of 28-CAP1. GM-CSF signal peptide bp 1-66; Anti-CD19 scFV bp 67-801; CD28 hinge and TM bp 808-1005; LAT 35-233 bp 1006-1605; linker bp 1606-1641; ZAP70 kinase domain bp 1642-2430.

SEQ ID NO: 19 is the amino acid sequence of 28-CAP2. GM-CSF signal peptide aa 1-22; Anti-CD19 scFV aa 23-267; CD28 hinge and TM aa 270-335; LAT 35-233 aa 336-534; linker aa 535-539; ZAP70 IB and kinase domain aa 540-885; ZAP70 601-619 aa 886-904.

SEQ ID NO: 20 is the nucleic acid sequence of 28-CAP2. GM-CSF signal peptide bp 1-66; Anti-CD19 scFV bp 67-801; CD28 hinge and TM bp 808-1005; LAT 35-233 bp 1006-1602; linker bp 1603-1617; ZAP70 IB and kinase domain bp 1618-2655; ZAP70 601-619 bp 2656-2712.

SEQ ID NO: 21 is the amino acid sequence of LAT-CAP3. GM-CSF signal peptide: amino acids 1-22; Anti-CD19 scFV: amino acids 23-267; LAT 1-35: amino acids 270-303; SLP76: amino acids 304-835; ZAP70 IB and kinase domain: amino acids 836-1181.

SEQ ID NO: 22 is the nucleic acid sequence of LAT-CAP3. GM-CSF signal peptide: nucleotides 1-66; Anti-CD19 scFV: nucleotides 67-801; LAT 1-35: nucleotides 808-909; SLP76: nucleotides 910-2505; ZAP70 IB and kinase domain: nucleotides 2506-3543.

SEQ ID NO: 23 is the amino acid sequence of 28-CAP4. GM-CSF signal peptide aa 1-22; Anti-CD19 scFV aa 23-267; CD28 hinge and TM aa 270-335; linker aa 336-340; ZAP70 IB and kinase domain aa 341-686; ZAP70 601-619 aa 687-705.

SEQ ID NO: 24 is the nucleic acid sequence of 28-CAP4. GM-CSF signal peptide bp 1-66; Anti-CD19 scFV bp 67-801; CD28 hinge and TM bp 808-1005; linker bp 1006-1020; ZAP70 IB and kinase domain bp 1021-2058; ZAP70 601-619 2059-2115.

SEQ ID NO: 25 is the amino acid sequence of 8-CAP2. GM-CSF signal peptide: amino acids 1-22; Anti-CD19 scFV: amino acids 23-267; CD8 hinge and TM: amino acids 268-336; LAT 34-233: amino acids 337-535; linker: amino acids 536-540; ZAP70 IB and kinase domain: amino acids 541-905.

SEQ ID NO: 26 is the nucleic acid sequence of 8-CAP2. GM-CSF signal peptide: nucleotides 1-66; Anti-CD19 scFV: nucleotides 67-801; CD8 hinge and TM: nucleotides 802-1008; LAT 34-233: nucleotides 1009-1605; linker: nucleotides 1606-1620; ZAP70 IB and kinase domain: nucleotides 1621-2715.

SEQ ID NO: 27 is the amino acid sequence of 8-CAP4. GM-CSF signal peptide: amino acids 1-22; Anti-CD19 scFV: amino acids 23-267; CD8 hinge and TM: amino acids 268-338; linker: amino acids 339-343; ZAP70 IB and kinase domain: amino acids 344-365.

SEQ ID NO: 28 is the nucleic acid sequence of 8-CAP4. GM-CSF signal peptide: nucleotides 1-66; Anti-CD19 scFV: nucleotides 67-801; CD8 hinge and TM: nucleotides 808-1014; linker: nucleotides 1015-1029; ZAP70 IB and kinase domain: nucleotides 1030-2124.

SEQ ID NO: 29 is the amino acid sequence of 8-CAP2 2Ya. GM-CSF signal peptide: amino acids 1-22; Anti-CD19 scFV: amino acids 23-267; CD8 hinge and TM: amino acids 268-336; LAT 34-233 Y132, Y171: amino acids 337-535; linker: amino acids 536-540; ZAP70 IB and kinase domain: amino acids 541-905.

SEQ ID NO: 30 is the nucleic acid sequence of 8-CAP2 2Ya. GM-CSF signal peptide: nucleotides 1-66; Anti-CD19 scFV: nucleotides 67-801; CD8 hinge and TM: nucleotides 802-1008; LAT 34-233 Y132, Y171: nucleotides 1009-1605; linker: nucleotides 1606-1620; ZAP70 IB and kinase domain: nucleotides 1621-2715.

SEQ ID NO: 31 is the amino acid sequence of 8-CAP2 2Yb. GM-CSF signal peptide: amino acids 1-22; Anti-CD19 scFV: amino acids 23-267; CD8 hinge and TM: amino acids 268-336; LAT 34-233 Y132, Y191: amino acids 337-535; linker: amino acids 536-540; ZAP70 IB and kinase domain: amino acids 541-905.

SEQ ID NO: 32 is the nucleic acid sequence of 8-CAP2 2Yb. GM-CSF signal peptide: nucleotides 1-66; Anti-CD19 scFV: nucleotides 67-801; CD8 hinge and TM: nucleotides 802-1008; LAT 34-233 Y132, Y191: nucleotides 1009-1605; linker: nucleotides 1606-1620; ZAP70 IB and kinase domain: nucleotides 1621-2715.

SEQ ID NO: 33 is the amino acid sequence of 8-CAP2 3Ya. GM-CSF signal peptide: amino acids 1-22; Anti-CD19 scFV: amino acids 23-267; CD8 hinge and TM: amino acids 268-336; LAT 34-233 Y132, Y171, Y191: amino acids 337-535; linker: amino acids 536-540; ZAP70 IB and kinase domain: amino acids 541-905.

SEQ ID NO: 34 is the nucleic acid sequence of 8-CAP2 3Ya. GM-CSF signal peptide: nucleotides 1-66; Anti-CD19 scFV: nucleotides 67-801; CD8 hinge and TM: nucleotides 802-1008; LAT 34-233 Y132, Y171, Y191: nucleotides 1009-1605; linker: nucleotides 1606-1620; ZAP70 IB and kinase domain: nucleotides 1621-2715.

SEQ ID NO: 35 is the amino acid sequence of 8-CAP2 3Yb. GM-CSF signal peptide: amino acids 1-22; Anti-CD19 scFV: amino acids 23-267; CD8 hinge and TM: amino acids 268-336; LAT 34-233 Y132, Y171, Y226: amino acids 337-535; linker: amino acids 536-540; ZAP70 IB and kinase domain: amino acids 541-905.

SEQ ID NO: 36 is the nucleic acid sequence of 8-CAP2 3Yb. GM-CSF signal peptide: nucleotides 1-66; Anti-CD19 scFV: nucleotides 67-801; CD8 hinge and TM: nucleotides 802-1008; LAT 34-233 Y132, Y171, Y226: nucleotides 1009-1605; linker: nucleotides 1606-1620; ZAP70 IB and kinase domain: nucleotides 1621-2715.

SEQ ID NO: 37 is the amino acid sequence of 8-CAP2 3Yc. GM-CSF signal peptide: amino acids 1-22; Anti-CD19 scFV: amino acids 23-267; CD8 hinge and TM: amino acids 268-336; LAT 34-233 Y132, Y191, Y226: amino acids 337-535; linker: amino acids 536-540; ZAP70 IB and kinase domain: amino acids 541-905.

SEQ ID NO: 38 is the nucleic acid sequence of 8-CAP2 3Yc. GM-CSF signal peptide: nucleotides 1-66; Anti-CD19 scFV: nucleotides 67-801; CD8 hinge and TM: nucleotides 802-1008; LAT 34-233 Y132, Y191, Y226: nucleotides 1009-1605; linker: nucleotides 1606-1620; ZAP70 IB and kinase domain: nucleotides 1621-2715.

SEQ ID NO: 39 is the amino acid sequence of 8-CAP4 ZAPAS1. GM-CSF signal peptide: amino acids 1-22; Anti-CD19 scFV: amino acids 23-267; CD8 hinge and TM: amino acids 268-338; linker: amino acids 339-343; ZAP70 IB and kinase domain M414A: amino acids 344-365.

SEQ ID NO: 40 is the nucleic acid sequence of 8-CAP4 ZAPAS1. GM-CSF signal peptide: nucleotides 1-66; Anti-CD19 scFV: nucleotides 67-801; CD8 hinge and TM: nucleotides 808-1014; linker: nucleotides 1015-1029; ZAP70 IB and kinase domain M414A: nucleotides 1030-2124.

SEQ ID NO: 41 is the amino acid sequence of 8-CAP4 ZAPAS2. GM-CSF signal peptide: amino acids 1-22; Anti-CD19 scFV: amino acids 23-267; CD8 hinge and TM: amino acids 268-338; linker: amino acids 339-343; ZAP70 IB and kinase domain C405V, M414A: amino acids 344-365.

SEQ ID NO: 42 is the nucleic acid sequence of 8-CAP4 ZAPAS2. GM-CSF signal peptide: nucleotides 1-66; Anti-CD19 scFV: nucleotides 67-801; CD8 hinge and TM: nucleotides 808-1014; linker: nucleotides 1015-1029; ZAP70 IB and kinase domain C405V, M414A: nucleotides 1030-2124.

SEQ ID NO: 43 is the amino acid sequence of 8-41BB CAP2. GM-CSF signal peptide: amino acids 1-22; Anti-CD19 scFV: amino acids 23-267; CD8 hinge and TM: amino acids 268-336; 41BB signaling domain: amino acids 337-378; LAT 35-233: amino acids 379-577; linker: amino acids 578-582; ZAP70 IB and kinase domain: amino acids 583-947.

SEQ ID NO: 44 is the nucleic acid sequence of 8-41BB CAP2. GM-CSF signal peptide: nucleotides 1-66; Anti-CD19 scFV: nucleotides 67-801; CD8 hinge and TM: nucleotides 802-1008; 41BB signaling domain: nucleotides 1009-1134; LAT 35-233: nucleotides 1135-1731; linker: nucleotides 1732-1746; ZAP70 IB and kinase domain: nucleotides 1747-2841.

SEQ ID NO: 45 is the amino acid sequence of 8-28 CAP2. GM-CSF signal peptide: amino acids 1-22; Anti-CD19 scFV: amino acids 23-267; CD8 hinge and TM: amino acids 268-336; CD28 signaling domain: amino acids 337-377; LAT 35-233: amino acids 378-576; linker: amino acids 577-581; ZAP70 IB and kinase domain: amino acids 582-946.

SEQ ID NO: 46 is the nucleic acid sequence of 8-28 CAP2. GM-CSF signal peptide: nucleotides 1-66; Anti-CD19 scFV: nucleotides 67-801; CD8 hinge and TM: nucleotides 802-1008; CD28 signaling domain: nucleotides 1009-1131; LAT 35-233: nucleotides 1132-1728; linker: nucleotides 1729-1743; ZAP70 IB and kinase domain: nucleotides 1744-2838.

SEQ ID NO: 47 is the amino acid sequence of 8-41BB CAP4. GM-CSF signal peptide: amino acids 1-22; Anti-CD19 scFV: amino acids 23-267; CD8 hinge and TM: amino acids 268-338; 41BB signaling domain: amino acids 339-380; linker: amino acids 381-385; ZAP70 IB and kinase domain: amino acids 386-750.

SEQ ID NO: 48 is the nucleic acid sequence of 8-41BB CAP4. GM-CSF signal peptide: nucleotides 1-66; Anti-CD19 scFV: nucleotides 67-801; CD8 hinge and TM: nucleotides 808-1014; 41BB signaling domain: nucleotides 1015-1140; linker: nucleotides 1141-1155; ZAP70 IB and kinase domain: nucleotides 1156-2250.

SEQ ID NO: 49 is the amino acid sequence of 8-28 CAP4. GM-CSF signal peptide: amino acids 1-22; Anti-CD19 scFV: amino acids 23-267; CD8 hinge and TM: amino acids 268-338; CD28 signaling domain: amino acids 339-379; linker: amino acids 380-384; ZAP70 IB and kinase domain: amino acids 385-749.

SEQ ID NO: 50 is the nucleic acid sequence of 8-28 CAP4. GM-CSF signal peptide: nucleotides 1-66; Anti-CD19 scFV: nucleotides 67-801; CD8 hinge and TM: nucleotides 808-1014; CD28 signaling domain: nucleotides 1015-1137; linker: nucleotides 1138-1152; ZAP70 IB and kinase domain: nucleotides 1153-2247.

SEQ ID NO: 51 is the amino acid sequence of CAP4.2. GMCSF signal peptide: amino acids 1-22; Anti-CD19 scFV: amino acids 23-265; CD28 hinge and TM: amino acids 270-335; linker: amino acids 336-340; ZAP70 IB+KD+end of protein: amino acids 341-705.

SEQ ID NO: 52 is the nucleic acid sequence of CAP4.2. GMCSF signal peptide: nucleotides 1-66; Anti-CD19 scFV: nucleotides 67-801; CD28 hinge and TM: nucleotides 808-1005; linker: nucleotides 1006-1020; ZAP70 IB+KD+end of protein: nucleotides 1021-2115.

SEQ ID NO: 53 is the amino acid sequence of CAP4.6. GMCSF signal peptide: amino acids 1-22; Anti-CD19 scFV: amino acids 23-265; CD28 hinge and TM: amino acids 270-335; CD28 intracellular domain: amino acids 336-376; ZAP70 IB+KD+end of protein: amino acids 377-741.

SEQ ID NO: 54 is the nucleic acid sequence of CAP4.6. GMCSF signal peptide: nucleotides 1-66; Anti-CD19 scFV: nucleotides 67-801; CD28 hinge and TM: nucleotides 808-1005; CD28 intracellular domain: nucleotides 1006-1128; ZAP70 IB+KD+end of protein: nucleotides 1129-2223.

SEQ ID NO: 55 is the amino acid sequence of CAP4.7. GMCSF signal peptide: amino acids 1-22; Anti-CD19 scFV: amino acids 23-265; CD28 hinge and TM: amino acids 270-335; CD28 intracellular domain: amino acids 336-376; full-length ZAP70: amino acids 377-995.

SEQ ID NO: 56 is the nucleic acid sequence of CAP4.7. GMCSF signal peptide: nucleotides 1-66; Anti-CD19 scFV: nucleotides 67-801; CD28 hinge and TM: nucleotides 808-1005; CD28 intracellular domain: nucleotides 1006-1128; ZAP70 full length: nucleotides 1129-2985.

SEQ ID NO: 57 is the amino acid sequence of CAP4.8. GMCSF signal peptide: amino acids 1-22; Anti-CD19 scFV: amino acids 23-265; CD28 hinge and TM: amino acids; mutated CD28 intracellular domain: amino acids 336-376; ZAP70 IB+KD+end of protein: amino acids 377-741.

SEQ ID NO: 58 is the nucleic acid sequence of CAP4.8. GMCSF signal peptide: nucleotides 1-66; Anti-CD19 scFV: nucleotides 67-801; CD28 hinge and TM: nucleotides 808-1005; CD28 mutated intracellular domain: nucleotides 1006-1128; ZAP70 IB+KD+end of protein: nucleotides 1129-2223.

SEQ ID NO: 59 is the amino acid sequence of CAP4.9. GMCSF signal peptide: amino acids 1-22; Anti-CD19 scFV: amino acids 23-265; CD28 hinge and TM: amino acids 270-335; mutated CD28 intracellular domain: amino acids 336-376; ZAP70 full-length: amino acids aa 377-995.

SEQ ID NO: 60 is the nucleic acid sequence of CAP4.9. GMCSF signal peptide: nucleotides 1-66; Anti-CD19 scFV: nucleotides 67-801; CD28 hinge and TM: nucleotides 808-1005; CD28 mutated intracellular domain: nucleotides 1006-1128; ZAP70 full length: nucleotides 1129-2985.

SEQ ID NO: 61 is the amino acid sequence of CAP4.10. GMCSF signal peptide: amino acids 1-22; Anti-CD19 scFV: amino acids 23-265; CD28 hinge and TM: amino acids 270-335; linker: amino acids 336-340; ZAP70 full-length: amino acids 341-959 SEQ ID NO: 62 is the nucleic acid sequence of CAP4.10. GMCSF signal peptide: nucleotides 1-66; Anti-CD19 scFV: nucleotides 67-801; CD28 hinge and TM: nucleotides 808-1005; linker: nucleotides 1006-1020; ZAP70 full length: nucleotides 1021-2880.

SEQ ID NO: 63 is the amino acid sequence of CAP4.11. GMCSF signal peptide: amino acids 1-22; Anti-CD19 scFV: amino acids 23-265; CD8 hinge and TM: amino acids 270-338; ZAP70 full length: amino acids 339-957.

SEQ ID NO: 64 is the nucleic acid sequence of CAP4.11. GMCSF signal peptide: nucleotides 1-66; Anti-CD19 scFV: nucleotides 67-801; CD8 hinge and TM: nucleotides 808-1014; ZAP70 full length: nucleotides 1015-2874.

SEQ ID NO: 65 is the amino acid sequence of CAP4.12. GMCSF signal peptide: amino acids 1-22; Anti-CD19 scFV: amino acids 23-265; CD8 hinge and TM: amino acids 270-338; 41BB signaling domain: amino acids 339-380; ZAP70 full-length: amino acids 381-999.

SEQ ID NO: 66 is the nucleic acid sequence of CAP4.12. GMCSF signal peptide: nucleotides 1-66; Anti-CD19 scFV: nucleotides 67-801; CD8 hinge and TM: nucleotides 808-1014; 41BB signaling domain: nucleotides 1015-1140; ZAP70 full length: nucleotides 1141-3000.

SEQ ID NOs: 67-69 are exemplary CD28 regulatory sequences.

SEQ ID NO: 70 is the amino acid sequence of 28-CAP2-2. GM-CSF signal peptide aa 1-22; Anti-CD19 scFV aa 23-267; CD28 hinge and TM aa 270-335; LAT 35-233 aa 336-534; linker aa 535-539; ZAP70 IB and kinase domain aa 540-885; ZAP70 601-619 aa 886-904.

SEQ ID NO: 71 is the nucleic acid sequence of 28-CAP2-2. GM-CSF signal peptide bp 1-66; Anti-CD19 scFV bp 67-801; CD28 hinge and TM bp 808-1005; LAT 35-233 bp 1006-1602; linker bp 1603-1617; ZAP70 IB and kinase domain bp 1618-2655; ZAP70 601-619 bp 2656-2712.

SEQ ID NO: 72 is the amino acid sequence of LAT-CAP3-2. GM-CSF signal peptide: amino acids 1-22; Anti-CD19 scFV: amino acids 23-267; LAT 1-35: amino acids 270-303; SLP76: amino acids 304-835; ZAP70 IB and kinase domain: amino acids 836-1181.

SEQ ID NO: 73 is the nucleic acid sequence of LAT-CAP3-2. GM-CSF signal peptide: nucleotides 1-66; Anti-CD19 scFV: nucleotides 67-801; LAT 1-35: nucleotides 808-909; SLP76: nucleotides 910-2505; ZAP70 IB and kinase domain: nucleotides 2506-3543.

SEQ ID NO: 74 is the amino acid sequence of 8-CAP2-2. GM-CSF signal peptide: amino acids 1-22; Anti-CD19 scFV: amino acids 23-267; CD8 hinge and TM: amino acids 268-336; LAT 34-233: amino acids 337-535; linker: amino acids 536-540; ZAP70 IB and kinase domain: amino acids 541-905.

SEQ ID NO: 75 is the nucleic acid sequence of 8-CAP2-2. GM-CSF signal peptide: nucleotides 1-66; Anti-CD19 scFV: nucleotides 67-801; CD8 hinge and TM: nucleotides 802-1008; LAT 34-233: nucleotides 1009-1605; linker: nucleotides 1606-1620; ZAP70 IB and kinase domain: nucleotides 1621-2715.

SEQ ID NO: 76 is the amino acid sequence of 8-CAP4-2. GM-CSF signal peptide: amino acids 1-22; Anti-CD19 scFV: amino acids 23-267; CD8 hinge and TM: amino acids 268-338; linker: amino acids 339-343; ZAP70 IB and kinase domain: amino acids 344-365.

SEQ ID NO: 77 is the nucleic acid sequence of 8-CAP4-2. GM-CSF signal peptide: nucleotides 1-66; Anti-CD19 scFV: nucleotides 67-801; CD8 hinge and TM: nucleotides 808-1014; linker: nucleotides 1015-1029; ZAP70 IB and kinase domain: nucleotides 1030-2124.

SEQ ID NO: 78 is the amino acid sequence of 8-CAP2 2Ya-2. GM-CSF signal peptide: amino acids 1-22; Anti-CD19 scFV: amino acids 23-267; CD8 hinge and TM: amino acids 268-336; LAT 34-233 Y132, Y171: amino acids 337-535; linker: amino acids 536-540; ZAP70 IB and kinase domain: amino acids 541-905.

SEQ ID NO: 79 is the nucleic acid sequence of 8-CAP2 2Ya-2. GM-CSF signal peptide: nucleotides 1-66; Anti-CD19 scFV: nucleotides 67-801; CD8 hinge and TM: nucleotides 802-1008; LAT 34-233 Y132, Y171: nucleotides 1009-1605; linker: nucleotides 1606-1620; ZAP70 IB and kinase domain: nucleotides 1621-2715.

SEQ ID NO: 80 is the amino acid sequence of 8-CAP2 2Yb-2. GM-CSF signal peptide: amino acids 1-22; Anti-CD19 scFV: amino acids 23-267; CD8 hinge and TM: amino acids 268-336; LAT 34-233 Y132, Y191: amino acids 337-535; linker: amino acids 536-540; ZAP70 IB and kinase domain: amino acids 541-905.

SEQ ID NO: 81 is the nucleic acid sequence of 8-CAP2 2Yb-2. GM-CSF signal peptide: nucleotides 1-66; Anti-CD19 scFV: nucleotides 67-801; CD8 hinge and TM: nucleotides 802-1008; LAT 34-233 Y132, Y191: nucleotides 1009-1605; linker: nucleotides 1606-1620; ZAP70 IB and kinase domain: nucleotides 1621-2715.

SEQ ID NO: 82 is the amino acid sequence of 8-CAP2 3Ya-2. GM-CSF signal peptide: amino acids 1-22; Anti-CD19 scFV: amino acids 23-267; CD8 hinge and TM: amino acids 268-336; LAT 34-233 Y132, Y171, Y191: amino acids 337-535; linker: amino acids 536-540; ZAP70 IB and kinase domain: amino acids 541-905.

SEQ ID NO: 83 is the nucleic acid sequence of 8-CAP2 3Ya-2. GM-CSF signal peptide: nucleotides 1-66; Anti-CD19 scFV: nucleotides 67-801; CD8 hinge and TM: nucleotides 802-1008; LAT 34-233 Y132, Y171, Y191: nucleotides 1009-1605; linker: nucleotides 1606-1620; ZAP70 IB and kinase domain: nucleotides 1621-2715.

SEQ ID NO: 84 is the amino acid sequence of 8-CAP2 3Yb-2. GM-CSF signal peptide: amino acids 1-22; Anti-CD19 scFV: amino acids 23-267; CD8 hinge and TM: amino acids 268-336; LAT 34-233 Y132, Y171, Y226: amino acids 337-535; linker: amino acids 536-540; ZAP70 IB and kinase domain: amino acids 541-905.

SEQ ID NO: 85 is the nucleic acid sequence of 8-CAP2 3Yb-2. GM-CSF signal peptide: nucleotides 1-66; Anti-CD19 scFV: nucleotides 67-801; CD8 hinge and TM: nucleotides 802-1008; LAT 34-233 Y132, Y171, Y226: nucleotides 1009-1605; linker: nucleotides 1606-1620; ZAP70 IB and kinase domain: nucleotides 1621-2715.

SEQ ID NO: 86 is the amino acid sequence of 8-CAP2 3Yc-2. GM-CSF signal peptide: amino acids 1-22; Anti-CD19 scFV: amino acids 23-267; CD8 hinge and TM: amino acids 268-336; LAT 34-233 Y132, Y191, Y226: amino acids 337-535; linker: amino acids 536-540; ZAP70 IB and kinase domain: amino acids 541-905.

SEQ ID NO: 87 is the nucleic acid sequence of 8-CAP2 3Yc-2. GM-CSF signal peptide: nucleotides 1-66; Anti-CD19 scFV: nucleotides 67-801; CD8 hinge and TM: nucleotides 802-1008; LAT 34-233 Y132, Y191, Y226: nucleotides 1009-1605; linker: nucleotides 1606-1620; ZAP70 IB and kinase domain: nucleotides 1621-2715.

SEQ ID NO: 88 is the amino acid sequence of 8-CAP4 ZAPAS1-2. GM-CSF signal peptide: amino acids 1-22; Anti-CD19 scFV: amino acids 23-267; CD8 hinge and TM: amino acids 268-338; linker: amino acids 339-343; ZAP70 IB and kinase domain M414A: amino acids 344-708.

SEQ ID NO: 89 is the nucleic acid sequence of 8-CAP4 ZAPAS1-2. GM-CSF signal peptide: nucleotides 1-66; Anti-CD19 scFV: nucleotides 67-801; CD8 hinge and TM: nucleotides 808-1014; linker: nucleotides 1015-1029; ZAP70 IB and kinase domain M414A: nucleotides 1030-2124.

SEQ ID NO: 90 is the amino acid sequence of 8-CAP4 ZAPAS2-2. GM-CSF signal peptide: amino acids 1-22; Anti-CD19 scFV: amino acids 23-267; CD8 hinge and TM: amino acids 268-338; linker: amino acids 339-343; ZAP70 IB and kinase domain C405V, M414A: amino acids 344-708.

SEQ ID NO: 91 is the nucleic acid sequence of 8-CAP4 ZAPAS2-2. GM-CSF signal peptide: nucleotides 1-66; Anti-CD19 scFV: nucleotides 67-801; CD8 hinge and TM: nucleotides 808-1014; linker: nucleotides 1015-1029; ZAP70 IB and kinase domain C405V, M414A: nucleotides 1030-2124.

SEQ ID NO: 92 is the amino acid sequence of 8-41BB CAP2-2. GM-CSF signal peptide: amino acids 1-22; Anti-CD19 scFV: amino acids 23-267; CD8 hinge and TM: amino acids 268-336; 41BB signaling domain: amino acids 337-378; LAT 35-233: amino acids 379-577; linker: amino acids 578-582; ZAP70 IB and kinase domain: amino acids 583-947.

SEQ ID NO: 93 is the nucleic acid sequence of 8-41BB CAP2-2. GM-CSF signal peptide: nucleotides 1-66; Anti-CD19 scFV: nucleotides 67-801; CD8 hinge and TM: nucleotides 802-1008; 41BB signaling domain: nucleotides 1009-1134; LAT 35-233: nucleotides 1135-1731; linker: nucleotides 1732-1746; ZAP70 IB and kinase domain: nucleotides 1747-2841.

SEQ ID NO: 94 is the amino acid sequence of 8-28 CAP2-2. GM-CSF signal peptide: amino acids 1-22; Anti-CD19 scFV: amino acids 23-267; CD8 hinge and TM: amino acids 268-336; CD28 signaling domain: amino acids 337-377; LAT 35-233: amino acids 378-576; linker: amino acids 577-581; ZAP70 IB and kinase domain: amino acids 582-946.

SEQ ID NO: 95 is the nucleic acid sequence of 8-28 CAP2-2. GM-CSF signal peptide: nucleotides 1-66; Anti-CD19 scFV: nucleotides 67-801; CD8 hinge and TM: nucleotides 802-1008; CD28 signaling domain: nucleotides 1009-1131; LAT 35-233: nucleotides 1132-1728; linker: nucleotides 1729-1743; ZAP70 IB and kinase domain: nucleotides 1744-2838.

SEQ ID NO: 96 is the amino acid sequence of 8-41BB CAP4-2. GM-CSF signal peptide: amino acids 1-22; Anti-CD19 scFV: amino acids 23-267; CD8 hinge and TM: amino acids 268-338; 41BB signaling domain: amino acids 339-380; linker: amino acids 381-385; ZAP70 IB and kinase domain: amino acids 386-750.

SEQ ID NO: 97 is the nucleic acid sequence of 8-41BB CAP4-2. GM-CSF signal peptide: nucleotides 1-66; Anti-CD19 scFV: nucleotides 67-801; CD8 hinge and TM: nucleotides 808-1014; 41BB signaling domain: nucleotides 1015-1140; linker: nucleotides 1141-1155; ZAP70 IB and kinase domain: nucleotides 1156-2250.

SEQ ID NO: 98 is the amino acid sequence of 8-28 CAP4-2. GM-CSF signal peptide: amino acids 1-22; Anti-CD19 scFV: amino acids 23-267; CD8 hinge and TM: amino acids 268-338; CD28 signaling domain: amino acids 339-379; linker: amino acids 380-384; ZAP70 IB and kinase domain: amino acids 385-749.

SEQ ID NO: 99 is the nucleic acid sequence of 8-28 CAP4-2. GM-CSF signal peptide: nucleotides 1-66; Anti-CD19 scFV: nucleotides 67-801; CD8 hinge and TM: nucleotides 808-1014; CD28 signaling domain: nucleotides 1015-1137; linker: nucleotides 1138-1152; ZAP70 IB and kinase domain: nucleotides 1153-2247.

SEQ ID NO: 100 is the amino acid sequence of LAT-CAP2-2. GM-CSF Signal peptide: amino acids 1-22; Anti-CD19scFV: amino acids 23-267; LAT full length 2-233: amino acids 271-502 (amino acids 271-273, LAT extracellular/hinge; amino acids 274-295, LAT TM; amino acids 296-502, LAT intracellular domain); linker: amino acids 503-521 (amino acids 504-519 are amino acids 318-333 of ZAP70); ZAP70-IB: amino acids 522-604; ZAP70-KD: amino acids 605-867.

SEQ ID NO: 101 is the nucleic acid sequence encoding LAT-CAP2-2. GM-CSF Signal peptide: nucleotides 1-66; Anti-CD19scFV: nucleotides 67-801; LAT full length 2-233: nucleotides 811-1506 (nucleotides 811-819, LAT extracellular/hinge; nucleotides 820-885, LAT TM; nucleotides 886-1506, LAT intracellular domain); linker: nucleotides 1507-1563 (nucleotides 1510-1557 encode amino acids 318-333 of ZAP70); ZAP70-IB: nucleotides 1564-1812; ZAP70-KD: nucleotides 1813-2601.

### DETAILED DESCRIPTION

Disclosed herein are chimeric membrane-spanning molecules, which in some examples include at least a portion of the adaptor molecule Linker for Activation of T cells (LAT) and/or a ZAP70 kinase domain fused to an extracellular targeting domain. These chimeric molecules are referred to as chimeric adapter proteins (CAPs) and provide advantages over current chimeric antigen receptors, which utilize CD3 zeta as the intracellular signaling domain.

LAT is a scaffold for a number of key signaling and adaptor molecules involved in TCR signal transduction downstream of TCR ligation. Incorporation of the downstream signaling molecule LAT allows signaling through the CAP complex to circumvent regulatory and inhibitory mechanisms that target upstream kinases and phosphatases involved in TCR activation. In addition, directly triggering the downstream signaling cascade may cause a more potent activation of T cells, allowing greater sensitivity to extracellular stimulus. Furthermore, T cell exhaustion is mediated by PD1, which targets upstream TCR activation. Therefore, LAT-based CAP-expressing T cells may be more resistant to PD1-mediated T cell exhaustion.

ZAP70 is a cytoplasmic protein tyrosine kinase that plays a critical role in the events involved in initiating T-cell responses by the antigen receptor. ZAP70 is the proximal protein tyrosine kinase downstream of TCRzeta, and clustering ZAP70 via the extracellular domain could be an efficient way to allow linkage to downstream signaling pathways and at the same time circumvent the inhibitory mechanisms that target the upstream receptor. Disclosed herein are constructs that only contain ZAP70 (without LAT), which are expected to allow for titering down the signaling strength compared to CAP constructs containing LAT and ZAP.

Costimulatory signals are required to achieve robust chimeric antigen receptor (CAR) T cell expansion, function, persistence and antitumor activity. Thus, in some examples, intracellular signaling domains from costimulatory molecules, such as CD28 or 4-1BB are incorporated into the CAPs to provide similar improvements in CAP function.

### I. Terms

Unless otherwise noted, technical terms are used according to conventional usage. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, Proteomics and Informatics, 3^{rd} Edition, Springer, 2008 (ISBN: 1402067534), and other similar references.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety, as are the GenBank Accession numbers (for the sequences present on Mar. 15, 2019). In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Antibody: A polypeptide ligand comprising at least one variable region that recognizes and binds (such as specifically recognizes and specifically binds) an epitope of an antigen. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273, 927-948, 1997; the “Chothia” numbering scheme), and the ImMunoGeneTics (IMGT) database (see, Lefranc, Nucleic Acids Res 29:207-9, 2001; the “IMGT” numbering scheme). The Kabat and IMGT databases are maintained online.

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242:423-426, 1988; Huston et al., Proc. Natl. Acad. Sci., 85:5879-5883, 1988; Ahmad et al., Clin. Dev. Immunol., 2012, doi:10.1155/2012/980250; Marbry, IDrugs, 13:543-549, 2010). The intramolecular orientation of the V_{H}-domain and the V_{L}-domain in a scFv, is typically not decisive for scFvs. Thus, scFvs with both possible arrangements (V_{H}-domain-linker domain-V_{L}-domain; V_{L}-domain-linker domain-V_{H}-domain) may be used. In a dsFv the V_{H} and V_{L} have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Isolated: An “isolated” biological component, such as a nucleic acid, protein (including antibodies) or organelle, has been substantially separated or purified away from other biological components in the environment (such as a cell) in which the component naturally occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Linker for Activation of T cells (LAT): A transmembrane protein that is phosphorylated by ZAP70 upon TCR activation and acts as a scaffold for Src homology 2 (SH2) containing molecules, such as GRB2 and PLCγ1. The resulting complex then acts a scaffold for recruitment of additional downstream effectors involved in TCR signaling. Exemplary human LAT nucleic acid and amino acid sequences are disclosed herein and also include GenBank Accession Nos. NM_001014989, NM_014387, NM_001014987, and NM_001014988 (nucleic acid sequences) and NP_001014989, NP_055202, NP_001014987, and NP_001014988 (amino acid sequences).

Natural Killer (NK) cells: Cells of the immune system that kill target cells in the absence of a specific antigenic stimulus and without restriction according to MHC class. Target cells can be tumor cells or cells harboring viruses. NK cells are characterized by the presence of CD56 and the absence of CD3 surface markers. NK cells typically comprise approximately 10 to 15% of the mononuclear cell fraction in normal peripheral blood. Historically, NK cells were first identified by their ability to lyse certain tumor cells without prior immunization or activation. NK cells are thought to provide a “back up” protective mechanism against viruses and tumors that might escape the CTL response by down-regulating MHC class I presentation. In addition to being involved in direct cytotoxic killing, NK cells also serve a role in cytokine production, which can be important to control cancer and infection.

In some examples, a “modified NK cell” is a NK cell transduced with a heterologous nucleic acid (such as one or more of the nucleic acids or vectors disclosed herein) or expressing one or more heterologous proteins. The terms “modified NK cell” and “transduced NK cell” are used interchangeably in some examples herein.

Purified: The term purified does not require absolute purity; rather, it is intended as a relative term. Thus, for example, a purified protein or nucleic acid preparation is one in which the protein or nucleic acid is more enriched than the protein or nucleic acid is in its natural environment (e.g., within a cell). In one embodiment, a preparation is purified such that the protein or nucleic acid represents at least 50% of the total protein or nucleic acid content of the preparation. Substantial purification denotes purification from other proteins or cellular components. A substantially purified protein or nucleic acid is at least 60%, 70%, 80%, 90%, 95% or 98% pure. Thus, in one specific, non-limiting example, a substantially purified protein or nucleic acid is 90% free of other components.

Recombinant: A nucleic acid or protein that has a sequence that is not naturally occurring or has a sequence that is made by an artificial combination of two otherwise separated segments of sequence (e.g., a “chimeric” sequence). This artificial combination can be accomplished by chemical synthesis or by the artificial manipulation of isolated segments of nucleic acids, for example, by genetic engineering techniques.

SLP-76: Also known as lymphocyte cytosolic protein 2 (LCP2). An adapter protein that is phosphorylated upon T cell receptor activation. Exemplary human SLP-76 nucleic acid and amino acid sequences are disclosed herein and also include GenBank Accession No. NM_005565 (nucleic acid sequence) and NP_005556 (amino acid sequence).

Subject: A living multi-cellular vertebrate organism, a category that includes both human and veterinary subjects, including human and non-human mammals.

T cell: A white blood cell (lymphocyte) that is an important mediator of the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker.

In one embodiment, a CD8⁺ T cell is a cytotoxic T lymphocyte (CTL). In another embodiment, a CD8⁺ cell is a suppressor T cell.

Activated T cells can be detected by an increase in cell proliferation and/or expression of or secretion of one or more cytokines (such as IL-2, IL-4, IL-6, IFNγ, or TNFα). Activation of CD8+ T cells can also be detected by an increase in cytolytic activity in response to an antigen.

In some examples, a “modified T cell” is a T cell transduced with a heterologous nucleic acid (such as one or more of the nucleic acids or vectors disclosed herein) or expressing one or more heterologous proteins. The terms “modified T cell” and “transduced T cell” are used interchangeably in some examples herein.

Transduced or Transformed: A transformed cell is a cell into which a nucleic acid molecule has been introduced by molecular biology techniques. As used herein, the terms transduction and transformation encompass all techniques by which a nucleic acid molecule might be introduced into such a cell, including transfection with viral vectors, the use of plasmid vectors, and introduction of DNA by electroporation, lipofection, and particle gun acceleration.

Treating or ameliorating a disease: “Treating” refers to a therapeutic intervention that decreases or inhibits a sign or symptom of a disease or pathological condition after it has begun to develop, such as a reduction in tumor size or tumor burden. “Ameliorating” refers to the reduction in the number or severity of signs or symptoms of a disease, such as cancer.

Vector: A nucleic acid molecule that can be introduced into a host cell (for example, by transfection or transduction), thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. A replication deficient viral vector is a vector that requires complementation of one or more regions of the viral genome required for replication due to a deficiency in at least one replication-essential gene function.

ZAP70: A protein tyrosine kinase involved in T cell development and activation. ZAP70 is tyrosine phosphorylated following TCR stimulation as part of the TCR-mediated signaling.

Exemplary human ZAP70 nucleic acid and amino acid sequences are disclosed herein and also include GenBank Accession Nos. NM_001079 and NM_207519 (nucleic acid sequences) and NP_001070 and NP_997402 (amino acid sequences).

### II. Chimeric Adaptor Proteins and Nucleic Acids

Disclosed herein are recombinant chimeric adaptor proteins (CAPs) and nucleic acids encoding said CAPs. In some embodiments, the CAPs are chimeric polypeptides including (a) an extracellular targeting domain; (b) a transmembrane domain; (c) an intracellular Linker for Activation of T cells (LAT) domain or SLP-76 domain; and (d) an intracellular ZAP70 domain, wherein (a)-(d) are in N-terminal to C-terminal order. In further embodiments, the CAPs further include an intracellular signaling domain (such as a 41BB or CD28 intracellular signaling domain) that is C-terminal to the transmembrane domain and N-terminal to the LAT or SLP-76 domain. In some embodiments, the CAPs further include a hinge domain that is C-terminal to the extracellular targeting domain and N-terminal to the transmembrane domain. In additional embodiments, the CAPs further include a signal sequence domain that is N-terminal to the extracellular signaling domain. Linkers (e.g., spacers) may be present between any of the components of the disclosed CAPs, for example, to allow proper folding and/or function of the CAP.

In additional embodiments, the CAPs are chimeric polypeptides including (a) an extracellular targeting domain; (b) a transmembrane domain; and (c) a ZAP70 domain, wherein (a)-(c) are in N-terminal to C-terminal order. In some embodiments, the ZAP70 domain is full length ZAP70. In other embodiments, the ZAP70 domain is a ZAP70 kinase domain (KD) or a ZAP70 interdomain B (IB) domain and a ZAP70 KD. In some embodiments, the CAPs further include a 41BB or CD28 intracellular signaling domain that is C-terminal to the transmembrane domain and N-terminal to the ZAP70 domain. In some embodiments, the CAPs further include a hinge domain that is C-terminal to the extracellular targeting domain and N-terminal to the transmembrane domain. In additional embodiments, the CAPs further include a signal sequence domain that is N-terminal to the extracellular signaling domain. Linkers (e.g., spacers) may be present between any of the components of the disclosed CAPs, for example, to allow proper folding and/or function of the CAP.

Individual components, as well as exemplary CAPs, are discussed below.

A. Extracellular Region

The extracellular region of the disclosed CAPs includes the extracellular targeting domain. In some embodiments, the extracellular region also includes a signal sequence (such as a signal sequence and an extracellular targeting domain). In other examples, the extracellular region also includes a signal sequence and a hinge domain (such as a signal sequence, an extracellular targeting domain, and a hinge domain). In other embodiments, the CAP does not include a signal sequence, for example, when the signal sequence has been cleaved from the CAP extracellular region.

In some embodiments, the extracellular targeting domain is an antigen binding domain of an antibody (such as an antigen binding domain of a monoclonal antibody) that specifically binds a target protein on the surface of a cell of interest (such as a tumor cell). In some examples, the antigen binding domain can include a V_{H} and a V_{L} including the HCDR1, HCDR2, HCDR3, LCDR1, LCDR2, and LCDR3 of the V_{H} and V_{L}, respectively, that specifically binds the target protein. In other examples, the antigen binding domain can be an scFv that specifically binds the target protein.

In particular embodiments, the targeting domain is an antigen binding domain or scFv that binds to a target of interest, such as a tumor associated antigen. Any targeting domain can be inserted in the CAPs described herein. In some embodiments, the targeting domain binds to a protein expressed on a hematological malignancy or a solid tumor. In some non-limiting examples, the targeting domain binds to CD19 (such as an scFv that binds to CD19). Exemplary targets of the extracellular targeting domain and corresponding malignancies are shown in Table 1.

TABLE 1
Exemplary extracellular targeting domain targets and malignancies

| Target | Malignancies |
|---|---|
| CD19 | Acute lymphoblastic leukemia (ALL), refractory or |
| | relapsed ALL, leukemia, lymphoma, B-cell |
| | lymphoma, T-cell lymphoma, Non-Hodgkin |
| | lymphoma, Diffuse large B-cell lymphoma |
| | (DLBCL), Primary mediastinal B-cell lymphoma, |
| | High grade B cell lymphoma, follicular lymphoma, |
| | DLBCL that results from follicular lymphoma, |
| | Chronic lymphocytic leukemia (CLL), Small |
| | lymphocytic leukemia (SLL), B-cell Non-Hodgkin |
| | lymphoma, B-cell Acute lymphoblastic leukemia, |
| | hematological cancer, acute myelogenous (myeloid) |
| | leukemia (AML), solid cancer, B-cell marginal zone |
| | lymphoma, Hodgkin's lymphoma, myeloma, |
| | multiple myeloma (MM), mantle cell lymphoma, |
| | Waldenstrom's hypergammaglobulinaemia, brain |
| | cancer, pancreatic cancer, gastrointestinal cancer, |
| | stomach cancer |
| CD22 | ALL, lymphoma, Non-Hodgkin lymphoma, |
| | autoimmune disease, DLBCL, CLL, B-cell |
| | lymphoma, leukemia |
| CD2 | Hematological cancer |
| CD4 | Leukemia, T-cell peripheral lymphoma, T-cell |
| | lymphoma, myeloma, MM |
| CD28 | Myeloma, MM |
| TNF receptor superfamily member 8 | Anaplastic large cell lymphoma, Hodgkin's |
| | lymphoma, T-cell peripheral lymphoma, T-cell |
| | lymphoma, non-Hodgkin's lymphoma |
| TNF receptor superfamily member 9 | Hematological cancer |
| TNF receptor superfamily member 10b | Solid cancer |
| TNF receptor superfamily member 13B | Hematological cancer, myeloma, solid cancer, MM |
| TNF receptor superfamily member 13C | Autoimmune disease, leukemia, lymphoma |
| B cell maturation factor (BCMA; TNF | Myeloma, MM, myasthenia gravis, gastrointestinal |
| receptor superfamily member 17) | cancer, stomach cancer, liver cancer, B-cell |
| | lymphoma, esophageal cancer, pancreatic cancer |
| CD171 | Neuroblastoma |
| Epidermal growth factor receptor | Brain cancer, lung cancer, biliary cancer, non-small |
| (EGFR) | cell lung cancer (NSCLC), pancreatic cancer, renal |
| | cancer, liver cancer, colorectal cancer |
| Epidermal growth factor receptor variant | Glioblastoma |
| III (EGFRvIII) | |
| Interleukin 3 receptor subunit alpha | AML, Myelodysplastic syndrome, Blastic |
| (CD123; IL3Ra) | plasmacytoid dendritic cell neoplasm, Hodgkin's |
| | lymphoma, chronic myelogenous (myeloid) leukemia |
| | (CML), hairy cell leukemia, mastocytosis, ALL, |
| | leukemia |
| Interleukin 7 receptor | Cancer |
| Interleukin 12 receptor subunit beta 1 | Ovarian cancer, peritoneal cancer |
| Interleukin-13 receptor alpha (IL13Ra) | Glioblastoma |
| Interleukin 13 receptor subunit alpha 2 | Brain cancer |
| Mesothelin | Ovarian cancer, cervical cancer, breast cancer, |
| | Fallopian tube cancer, pancreatic cancer, lung cancer, |
| | colorectal cancer, peritoneal carcinoma, solid cancer, |
| | mesothelioma, endometrial cancer |
| Mucin 16 (MUC-16) | Ovarian cancer, Peritoneal cancer |
| Mucin 1 (Muc1) | Sarcoma, breast cancer, cervical cancer, pancreatic |
| | cancer, lung cancer, liver cancer, glioma, colorectal |
| | cancer, gastric cancer, brain cancer, gastrointestinal |
| | cancer, stomach cancer, NSCLC, myeloma, MM, |
| | ovarian cancer, ovarian cancer, renal cancer |
| Receptor tyrosine kinase like orphan | Breast cancer, ovarian cancer, lung adenocarcinoma, |
| receptor 1 (ROR-1) | lung cancer, lymphoblastic leukemia, CLL, ALL, |
| | NSCLC, mantle cell lymphoma |
| Prostate Stem Cell Antigen (PSCA) | Pancreatic cancer, lung cancer, prostate cancer, |
| | gastrointestinal cancer, stomach cancer, bladder |
| | cancer |
| CD33 | Myeloid leukemia, AML, hematological cancer, |
| | leukemia |
| Prostate specific membrane antigen | Prostate cancer, bladder cancer, cervical cancer |
| (PSMA) | |
| CD70 | B cell malignancies, breast cancer, ovarian cancer, |
| | pancreatic cancer, melanoma, renal cell cancer, B- |
| | cell lymphoma, T-cell lymphoma, renal cancer, non- |
| | Hodgkin's lymphoma |
| Human epidermal growth factor receptor | Breast cancer, ovarian cancer, lung cancer, gastric |
| 2 (HER2) | cancer, colorectal cancer, pancreatic cancer, |
| | glioblastoma, glioma |
| Carcinoembryonic antigen (CEA) | Breast cancer, lung cancer, colorectal cancer, gastric |
| | cancer, pancreatic cancer, liver metastases, liver |
| | cancer, peritoneal cancer, gastrointestinal cancer, |
| | stomach cancer |
| GTPase-activating protein (GAP) | Solid tumors |
| CD5 | T-cell ALL, T-cell non-Hodgkin lymphoma, T-cell |
| | peripheral lymphoma, leukemia, ALL, non- |
| | Hodgkin's lymphoma |
| CD38 | Myeloma, MM, AML |
| Ephrin type-A receptor 2 (EphA2) | Glioma |
| Fibroblast activation protein alpha | Mesothelioma |
| (FAP) | |
| Ganglioside G2 (GD2) | Glioma, neuroblastoma, sarcoma, cervical cancer, |
| | lung cancer, small cell lung cancer, melanoma, |
| | osteosarcoma |
| Epithelial cell adhesion molecule | Breast cancer, prostate cancer, colon cancer, |
| (EpCam) | pancreatic cancer, gastric cancer, hepatic carcinoma, |
| | esophageal carcinoma, lymphoma, leukemia, |
| | gastrointestinal cancer, stomach cancer, liver cancer, |
| | ovarian cancer |
| CD133 | AML, breast cancer, ovarian cancer, colorectal |
| | cancer, glioma, pancreatic cancer, liver cancer |
| Glypican 2 | Bladder cancer, small cell lung cancer, lung cancer, |
| | neuroblastoma |
| Glypican 3 (GPC3) | Lymphoma, leukemia, pancreatic cancer, colorectal |
| | cancer, lung cancer, liver cancer, brain cancer, breast |
| | cancer, gastrointestinal cancer, stomach cancer, |
| | NSCLC, squamous cell carcinoma |
| CD44 | AML, myeloma, MM, myeloma, breast cancer, |
| | colorectal cancer, lung cancer, leukemia |
| C-type lectin domain family 12 member | AML, hematological cancer |
| A (CLL1) | |
| C-type lectin domain containing 14A | Solid cancer |
| GDNF family receptor alpha 4 | Thyroid cancer |
| Membrane spanning 4-domains A1 | Leukemia, Lymphoma, ALL, CLL, B-cell |
| (CD20) | lymphoma, non-Hodgkin's lymphoma, DLBCL, |
| | melanoma, Waldenstrom's |
| | hypergammaglobulinaemia, brain cancer, pancreatic |
| | cancer |
| Programmed cell death 1 (PD-1) | Solid cancer, hematological cancer, esophageal |
| | cancer, DLBCL, non-Hodgkin's lymphoma, B-cell |
| | lymphoma |
| Erb-B2 receptor tyrosine kinase 2 | Breast cancer, brain cancer, osteosarcoma, sarcoma, |
| | ovarian cancer, colorectal cancer, gastrointestinal |
| | cancer, stomach cancer |
| Chondroitin sulfate proteoglycan 4 | Brain cancer, breast cancer, neuroblastoma |
| CD274 (PD-L1) | ALL, myeloma, MM, non-Hodgkin's lymphoma, |
| | pancreatic cancer, breast cancer, NSCLC, lung |
| | cancer |
| EPH receptor A2 | Solid cancer |
| EPH receptor A3 | Solid cancer |
| Folate hydrolase 1 (PSMA) | Prostate cancer |
| Folate receptor alpha | Ovarian cancer |
| Folate receptor beta | AML, solid cancer |
| Natural killer cell cytotoxicity receptor 3 | Hematological cancer, solid cancer |
| ligand 1 | |
| CD7 | AML, T-cell lymphoma, T-cell peripheral |
| | lymphoma, ALL, non-Hodgkin's lymphoma |
| KIT proto-oncogene, receptor tyrosine | Solid cancer |
| kinase | |
| Interleukin 6 | DLBCL |
| Interleukin 12A | Solid cancer |
| Interleukin 12B | Colorectal cancer |
| Interleukin 13 | Brain cancer |
| Interleukin 15 | CLL, B-cell lymphoma, hematological cancer |
| Fc fragment of IgE receptor II | B-cell lymphoma |
| Fc fragment of IgG receptor IIIa | Hematological cancer, solid cancer |
| Fms related tyrosine kinase 3 | Hematological cancer, AML, solid cancer |
| CD40 | B-cell lymphoma |
| Killer cell lectin like receptor K1 | Bladder cancer, breast cancer, colorectal cancer, |
| | fallopian tube cancer, AML, NSCLC, lung cancer, |
| | myeloma, ovarian cancer, pancreatic cancer, |
| | Myelodysplastic syndrome, hematological cancer |
| Cancer/testis antigen 1B | Solid cancer |
| MET proto-oncogene, receptor tyrosine | Solid cancer |
| kinase | |
| Thyroid stimulating hormone receptor | Thyroid cancer |
| (TSHR) | |
| T cell receptor beta constant 1 | Anaplastic large cell cancer, non-Hodgkin's |
| | lymphoma, T-cell lymphoma, T-cell peripheral |
| | lymphoma |
| T cell receptor beta constant 2 | T-cell lymphoma |
| AXL receptor tyrosine kinase | Renal cancer, bladder cancer, gastrointestinal cancer, |
| | stomach cancer, pancreatic cancer, soft tissue |
| | sarcoma |
| RAR related orphan receptor A | Renal cancer, bladder cancer, gastrointestinal cancer, |
| | stomach cancer, pancreatic cancer, soft tissue |
| | sarcoma |
| Alpha fetoprotein | Liver cancer |
| SLAM family member 7 (SLAMF7) | Myeloma, MM, B-cell lymphoma |
| Transforming growth factor beta | Prostate cancer |
| receptor 1 | |
| Transforming growth factor beta | Non-Hodgkin's lymphoma |
| receptor 2 | |
| Delta like canonical Notch ligand 3 | Small cell lung cancer, lung cancer |
| Claudin 3 | Colorectal cancer, ovarian cancer, pancreatic cancer, |
| | prostate cancer |
| Claudin 6 | Solid cancer |
| Claudin 18 | Gastrointestinal cancer, stomach cancer, pancreatic |
| | cancer, hematological cancer, ovarian cancer |
| Lewis Y (LeY) antigen | Lung cancer, NSCLC |
| L1 cell adhesion molecule (L1CAM) | Neuroblastoma, lung cancer, pancreatic cancer, renal |
| | cancer, brain cancer |
| Trophoblast glycoprotein | Solid cancer |
| Coagulation factor VIII | Hemophilia A |
| Leukocyte cell derived chemotaxin 2 | AL amyloidosis, LECT2 amyloidosis, transthyretin- |
| (LECT2) | related hereditary amyloidosis, transthyretin-related |
| | wild-type amyloidosis, amyloidosis |
| Transthyretin | AL amyloidosis, LECT2 amyloidosis, transthyretin- |
| | related hereditary amyloidosis, transthyretin-related |
| | wild-type amyloidosis, amyloidosis |
| Muscle associated receptor tyrosine | Myasthenia gravis |
| kinase (MuSK) | |
| CD160 | CLL |
| Enhancer of Zeste 2 polycomb | Solid cancer |
| repressive complex 2 subunit (ENX) | |
| Sialophorin (CD43) | Leukemia, T-cell lymphoma |
| HIV-1 Env | HIV infection, AIDS |
| Complement C3d receptor 2 (CR2) | Gastrointestinal cancer, stomach cancer, lymphoma, |
| | nasopharyngeal cancer, non-Hodgkin's lymphoma |
| Kappa myeloma antigen (KMA) | Myeloma, MM |
| Lambda myeloma antigen (LMA) | Myeloma, MM |
| Kinase insert domain receptor | Solid cancer |
| (VEGFR2) | |
| Intercellular adhesion molecule 1 | Thyroid cancer |
| (ICAM-1) | |
| Aspartate beta-hydroxylase (ASPH) | Hematological cancer, solid cancer |
| Prominin 1 | Brain cancer |
| Cadherin 17 | Colorectal cancer, liver cancer, pancreatic cancer |
| Tumor associated calcium signal | Colorectal cancer, liver cancer, pancreatic cancer |
| transducer 2 (Trop-2; EGP-1) | |
| TRAF interacting protein (TRAIP) | Solid cancer |
| Tyrosinase (TYR) | Solid cancer, melanoma |
| Desmoglein 1 | Pemphigus |
| Desmoglein 3 | Pemphigus |
| Integrin beta 7 | Myeloma, MM |
| Adhesion G protein coupled receptor E1 | AML, eosinophilic asthma, Churg-Strauss syndrome, |
| | eosinophilic esophagitis |
| Gonadotropin releasing hormone | Ovarian cancer, prostate cancer, pancreatic cancer |
| receptor | |
| Tumor-associated glycoprotein 72 | Colorectal cancer, ovarian cancer, prostate cancer |
| (TAG-72) | |

Thus, in some embodiments, the extracellular targeting domain binds to one or more of CD19, CD22, B cell maturation factor (BCMA), CD171, epidermal growth factor receptor variant III (EGFRvIIJ), interleukin-13 receptor alpha (IL-13Ra), mesothelin, mucin 16, mucin 1, receptor tyrosine kinase-like orphan receptor 1 (ROR-1), prostate stem cell antigen (PSCA), CD33, prostate-specific membrane antigen (PMSA), CD123, CD70, human epidermal growth factor receptor 2 (HER2), carcinoembryonic antigen (CEA), GPTase-activating protein (GAP), CD5, CD38, ephrin type-A receptor 2 (EphA2), fibroblast activation protein alpha (FAP), ganglioside G2 (GD2), epithelial cell adhesion molecule (EpCam), CD133, and glypican 3 (GPC3). This list is non-limiting, and additional extracellular targeting domains can also be utilized. For example, the extracellular domain may include any TCR, for example, a TCR clonally expressed in a leukemia or lymphoma.

In particular examples, the extracellular targeting region is a scFv that binds to CD19. In some examples, the CD19 scFv has an amino acid sequence at least 90%, at least 95%, at least 96%, at least 97%, at least 98% or at least 99% identical to, or including or consisting of amino acids 23-267 of SEQ ID NO: 17. In additional examples, the CD19 scFv is encoded by a nucleic acid molecule with at least 90%, at least 95%, at least 96%, at least 97%, at least 98% or at least 99% identical to, or including or consisting of nucleotides 67-801 of SEQ ID NO: 18.

In some embodiments, the extracellular region of the CAP includes a signal sequence domain, e.g., N-terminal to the targeting domain, for example, to facilitate expression of the CAP on the cell surface. In some examples, following expression of the CAP on the cell surface, the signal sequence domain may be cleaved off of the CAP. Therefore, in some embodiments, the CAP lacks a signal sequence domain.

The signal sequence domain can include any suitable signal peptide sequence. In one non-limiting example, the signal peptide domain is a human granulocyte-macrophage colony-stimulating factor (GM-CSF) signal sequence, such as an amino acid sequence at least 90%, at least 95%, at least 96%, at least 97%, at least 98% or at least 99% identical to, or including or consisting of amino acids 1-22 of SEQ ID NO: 17. In additional examples, the GM-CSF signal sequence is encoded by a nucleic acid molecule with at least 90%, at least 95%, at least 96%, at least 97%, at least 98% or at least 99% identical to, or including or consisting of nucleotides 1-66 of SEQ ID NO: 18. In additional examples, the signal peptide domain is a CD8 signal sequence, such as an amino acid sequence at least 90%, at least 95%, at least 96%, at least 97%, at least 98% or at least 99% identical to, or including or consisting of SEQ ID NO: 11. In some examples, the CD8 signal sequence is encoded by a nucleic acid molecule with at least 90%, at least 95%, at least 96%, at least 97%, at least 98% or at least 99% identical to, or including or consisting of SEQ ID NO: 12. However, other signal sequences known in the art can be utilized.

In some embodiments, the disclosed CAPs also include a hinge domain, which is in some examples a spacer between the extracellular targeting domain and the transmembrane region. However, in other embodiments, the CAP does not include a hinge domain. In other examples, the hinge domain is part of the transmembrane domain. For example, a LAT hinge domain (which is three amino acids—EEA) may be included as part of the LAT TM domain in some examples. The hinge domain of the CAP, if included, is C-terminal to the extracellular targeting domain and N-terminal to the transmembrane domain. In some examples, the hinge domain is about 3 to 250 amino acids long (such as about 3-25, 5-30, 10-50, 40-80, 60-100, 70-120, 90-140, 110-150, 125-160, 130-180, 150-200, 170-225, or 210-250 amino acids long). In some examples, a spacer or linker is included between the targeting domain and the hinge domain, for example a linker 2-4 amino acids long.

In some embodiments, the extracellular hinge domain is a CD28 hinge domain or a CD8 hinge domain. In some examples, the hinge domain is a CD28 hinge domain at least 90%, at least 95%, at least 96%, at least 97%, at least 98% or at least 99% identical to, or including or consisting of the amino acid sequence of amino acids 270-308 of SEQ ID NO: 17. In some examples, the CD28 hinge domain is encoded by a nucleic acid molecule with at least 90%, at least 95%, at least 96%, at least 97%, at least 98% or at least 99% identical to, or including or consisting of nucleotides 808-924 of SEQ ID NO: 18. In other embodiments, the hinge domain is a CD8 hinge domain at least 90%, at least 95%, at least 96%, at least 97%, at least 98% or at least 99% identical to, or including or consisting of the amino acid sequence of amino acids 266-312 of SEQ ID NO: 25. In some examples, the CD8 hinge domain is encoded by a nucleic acid molecule with at least 90%, at least 95%, at least 96%, at least 97%, at least 98% or at least 99% identical to, or including or consisting of nucleotides 796-936 of SEQ ID NO: 26. In other examples, the extracellular hinge domain is from an immunoglobulin such as an IgG1, IgG4, or IgD hinge domain.

B. Transmembrane Domain

The disclosed CAPs include a transmembrane domain that is linked to the hinge domain, if present (e.g., C-terminal to the hinge domain) and the LAT (e.g., N-terminal to the LAT). In other examples, the transmembrane is linked to the extracellular targeting domain (e.g., C-terminal to the extracellular targeting domain) and the LAT domain (e.g., N-terminal to the LAT domain), if a hinge domain is not present. In other embodiments, the transmembrane domain is linked to the hinge domain (e.g., C-terminal to the hinge domain, if present) and the ZAP70 domain (e.g., N-terminal to the ZAP70 domain) or the intracellular signaling domain (e.g., N-terminal to the intracellular signaling domain, if present).

The transmembrane domain can be from any membrane-bound or transmembrane protein, or could be synthetic. One of ordinary skill in the art can identify transmembrane sequences, for example using transmembrane domain prediction programs, such as TMpred (available at embnet.vital-it.ch/software/TMPRED_form.html), TMHMM v.2.0 (available at cbs.dtu.dk/services/TMHMM-2.0/), and other publicly available prediction programs.

In some embodiments, the transmembrane domain of the disclosed CAPs is a transmembrane domain from CD8, CD28, LAT, CD3 epsilon, CD45, CD4, CD5, CD8, CD9, CD16, CD22, CD33, CD37, CD64, CD80, CD86, CD134, CD137, or CD154. In other embodiments, the transmembrane domain is a transmembrane domain from a T cell receptor (TCR), such as TCRα, TCRβ, or TCRζ chain.

In particular examples, the transmembrane domain is the transmembrane domain of CD8, for example, a transmembrane domain at least 90%, at least 95%, at least 96%, at least 97%, at least 98% or at least 99% identical to, or including or consisting of the amino acid sequence of amino acids 313-336 of SEQ ID NO: 25. In some examples, the CD8 transmembrane domain is encoded by a nucleic acid molecule with at least 90%, at least 95%, at least 96%, at least 97%, at least 98% or at least 99% identical to, or including or consisting of nucleotides 937-1008 of SEQ ID NO: 26. In other examples, the transmembrane domain is the transmembrane domain of CD28, for example, a transmembrane domain at least 90%, at least 95%, at least 96%, at least 97%, at least 98% or at least 99% identical to, or including or consisting of the amino acid sequence of amino acids 309-335 of SEQ ID NO: 17. In some examples, the CD28 transmembrane domain is encoded by a nucleic acid molecule with at least 90%, at least 95%, at least 96%, at least 97%, at least 98% or at least 99% identical to, or including or consisting of nucleotides 925-1005 of SEQ ID NO: 18.

In yet other examples, the transmembrane domain is the transmembrane domain of LAT, for example, a transmembrane domain at least 90%, at least 95%, at least 96%, at least 97%, at least 98% or at least 99% identical to, or including or consisting of the amino acid sequence of amino acids 271-295 of SEQ ID NO: 15, wherein the first three amino acids are a LAT hinge domain. In some examples, the LAT hinge and transmembrane domain is encoded by a nucleic acid molecule with at least 90%, at least 95%, at least 96%, at least 97%, at least 98% or at least 99% identical to, or including or consisting of nucleotides 811-885 of SEQ ID NO: 16.

In particular non-limiting examples, when the transmembrane domain is from CD8, the hinge domain is also from CD8 or when the transmembrane domain is from CD28, the hinge domain is also from CD28. In other examples, the transmembrane domain and the hinge domain may be from different sources, for example a combination of a hinge domain and a transmembrane domain from any of CD8, CD28, and LAT. In some non-limiting examples, the transmembrane domain is from CD8 and the hinge domain is from CD28, the transmembrane domain is from CD28 and the hinge domain is from CD8, or the transmembrane domain is from LAT and the hinge domain is from CD8 or CD28.

C. LAT Domain

In some embodiments, the disclosed CAPs include an intracellular LAT domain that is C-terminal of the transmembrane domain. In some examples, a spacer may be included between the TM domain and the LAT domain. Current CARs utilize CD3ζ as the signaling domain with varied combinations of co-stimulatory, transmembrane, hinge, and extracellular targeting domains. LAT serves as a key scaffold for a number of key signaling and adaptor molecules involved in TCR signal transduction downstream of TCR ligation. After TCR activation, LAT forms a distinct signaling complex through a crosslinking mechanism mediated by GRB2 and SOS. In addition, recruitment of the LAT complex is kinetically regulated by co-stimulatory and inhibitory molecules and its formation is sufficient to cause full T cell activation independent of TCR activation. Thus, in one aspect the CAPs disclosed herein differ from CARs in that the LAT domain replaces CD3ζ as the signaling domain.

The LAT domain may include all or a portion of the LAT protein. In some examples, the CAP includes full-length LAT, such as an amino acid sequence at least 90%, at least 95%, at least 96%, at least 97%, at least 98%, or at least 99% identical to, or including or consisting of the amino acid sequence of amino acids 2-233 of LAT, such as amino acids 276-507 of SEQ ID NO: 5. The full-length LAT includes the extracellular domain, TM domain and intracellular domains of LAT. In some examples, the LAT domain is encoded by a nucleic acid molecule with at least 90%, at least 95%, at least 96%, at least 97%, at least 98% or at least 99% identical to, or including or consisting of nucleotides 826-1521 of SEQ ID NO: 6. In particular non-limiting examples, the LAT domain is at least 90%, at least 95%, at least 96%, at least 97%, at least 98%, or at least 99% identical to, or includes or consists of amino acids 34-233 of LAT, such as amino acids 336-535 of SEQ ID NO: 17.

In other examples the LAT domain encodes amino acids 34-233 of LAT and is a nucleic acid with at least 90%, at least 95%, at least 96%, at least 97%, at least 98%, or at least 99% identical to, or includes or consists of nucleotides 1006-1605 of SEQ ID NO: 18. Amino acids 34-233 correspond to the intracellular domain of LAT, and is used for example, when hinge and TM domains from another source are used in the CAP construct.

In additional embodiments, the LAT domain includes one or more mutations, for example to improve protein stability, expression, and/or signaling functions. In one example, the LAT domain includes K52R and/or K204R substitutions. See, e.g., U.S. Pat. No. 8,779,095, incorporated herein by reference.

In other embodiments, the LAT domain includes one or more mutations of a tyrosine residue. In some embodiments, the LAT domain includes only two or three of the ten cytosolic tyrosine residues present in the wild type LAT domain. The LAT cytosolic domain contains ten tyrosines, of which all are not needed for LAT function. The four membrane-distal tyrosine residues Y132, Y171, Y191 and Y226 have been shown to be most important for TCR-mediated signaling (e.g., Zhang et al., J. Biol. Chem. 275:23355-23361, 2000). Thus, LAT mutants with two or three of these four membrane-distal tyrosines intact might allow for titering down of signaling strength from LAT-containing CAPs. Thus, in some examples, the CAPs include a LAT domain in which six or more (for example, 6, 7, 8, 9, or 10) of the tyrosine residues are substituted with phenylalanines. In some examples, the LAT domain only includes tyrosines at amino acids Y132, Y171, Y191, or Y226 of the LAT domain (e.g., corresponding to amino acid positions 434, 473, 493, and 528, respectively of SEQ ID NO: 25), or a combination of two or more thereof, with other tyrosine residues in the LAT domain substituted for phenylalanine. In some examples, the LAT domain includes tyrosine at amino acids corresponding to Y434 and Y473 of SEQ ID NO: 29, with other tyrosine residues in the LAT domain substituted for phenylalanine. In other examples, the LAT domain includes tyrosine at amino acids corresponding to Y434 and Y493 of SEQ ID NO: 31, with other tyrosine residues in the LAT domain substituted for phenylalanine. In further examples, the LAT domain includes tyrosine at amino acids corresponding to Y434, Y473, and Y493 of SEQ ID NO: 33, with other tyrosine residues in the LAT domain substituted for phenylalanine. In additional examples, the LAT domain includes tyrosine at amino acids corresponding to Y434, Y473, and Y538 of SEQ ID NO: 528, with other tyrosine residues in the LAT domain substituted for phenylalanine. In other examples, the LAT domain includes tyrosine at amino acids corresponding to Y434, Y493, and Y528 of SEQ ID NO: 37, with other tyrosine residues in the LAT domain substituted for phenylalanine.

In other embodiments, the LAT domain is replaced with an SLP-76 domain. Upon TCR activation, ZAP-70 phosphorylates SLP-76 and LAT and both SLP-76 and LAT are involved in T cell signaling. A previous study showed that an important role for LAT is to recruit SLP-76 and its associated molecules to the membrane, where signaling molecules are concentrated. This study showed that the LAT cytosolic domain could be replaced by SLP-76 (Boerth et al., J Exp Med 192(7):1047-1058, 2000). Therefore, including SLP-76 in the CAPs described herein could enhance signaling in a T cell.

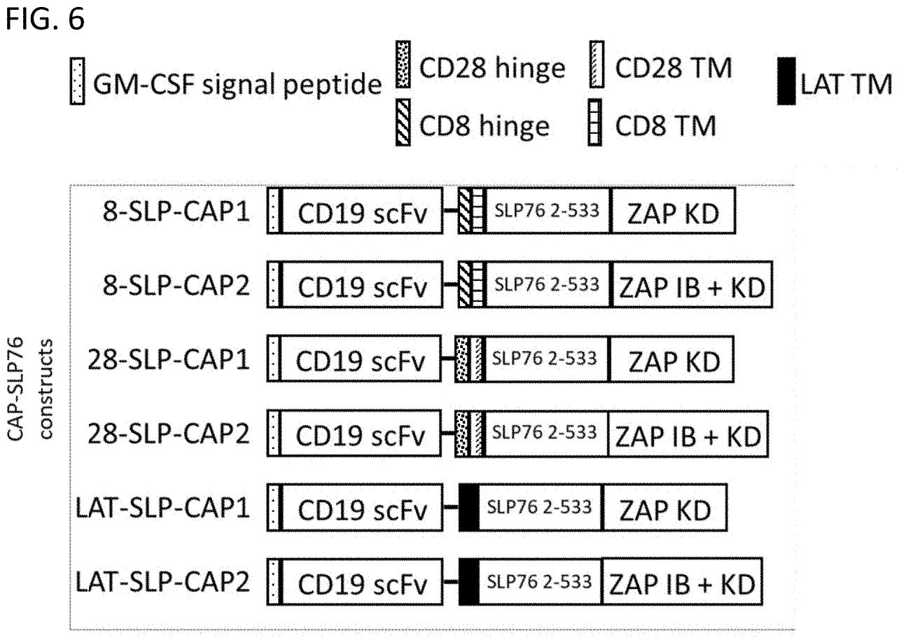

Thus, in some examples, the LAT domain in the CAPs described herein is replaced with an SLP-76 domain. Exemplary CAP constructs including SLP-76 (CAP-SLP76 constructs) are shown in FIG. 6. In some examples, the SLP-76 domain may include all or a portion of the SLP-76 protein. In some examples, the CAP includes full-length SLP-76 (excluding the starting methionine), such as an amino acid sequence at least 90%, at least 95%, at least 96%, at least 97%, at least 98%, or at least 99% identical to, or including or consisting of the amino acid sequence of amino acids 2-533 of SEQ ID NO: 14 or amino acids 305-836 of SEQ ID NO: 16. In some examples, the SLP-76 domain is encoded by a nucleic acid molecule with at least 90%, at least 95%, at least 96%, at least 97%, at least 98% or at least 99% identical to, or including or consisting of nucleotides 4-1602 of SEQ ID NO: 13 or nucleotides 913-2508 of SEQ ID NO: 15.

D. ZAP70 Domain

The disclosed CAPs also include at least a portion of a ZAP70 protein. In some embodiments, the ZAP70 domain is located C-terminal to the LAT domain. In other embodiments, the ZAP70 domain is C-terminal to the transmembrane domain or C-terminal to additional domains (such as a 41BB or CD28 intracellular domain), for example in embodiments that do not include a LAT domain.

ZAP70 is a protein tyrosine kinase, which upon T cell stimulation through the T cell antigen receptor, plays a critical role in T cell signaling. Activated ZAP70 phosphorylates adapters LAT and SLP-76, which then function as scaffolds for various adapters and enzymes. Together these signaling molecules lead to T cell activation. Thus, a fusion of LAT and ZAP-70 kinase domain is expected to bypass TCR activation. In some embodiments, the ZAP-70 IB domain is included because the ZAP70 IB domain contains tyrosine residues that play a role in the regulation of ZAP70 activity.

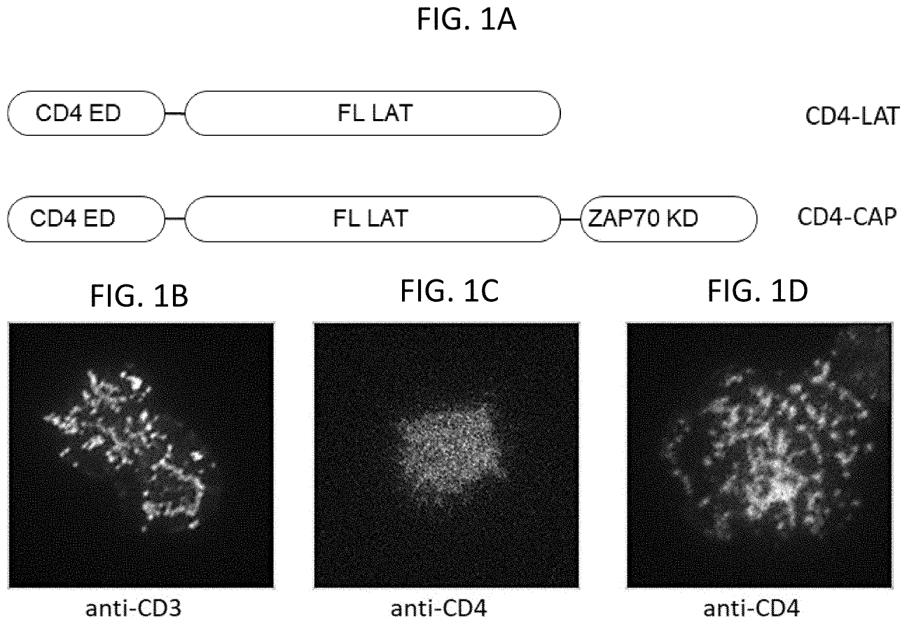

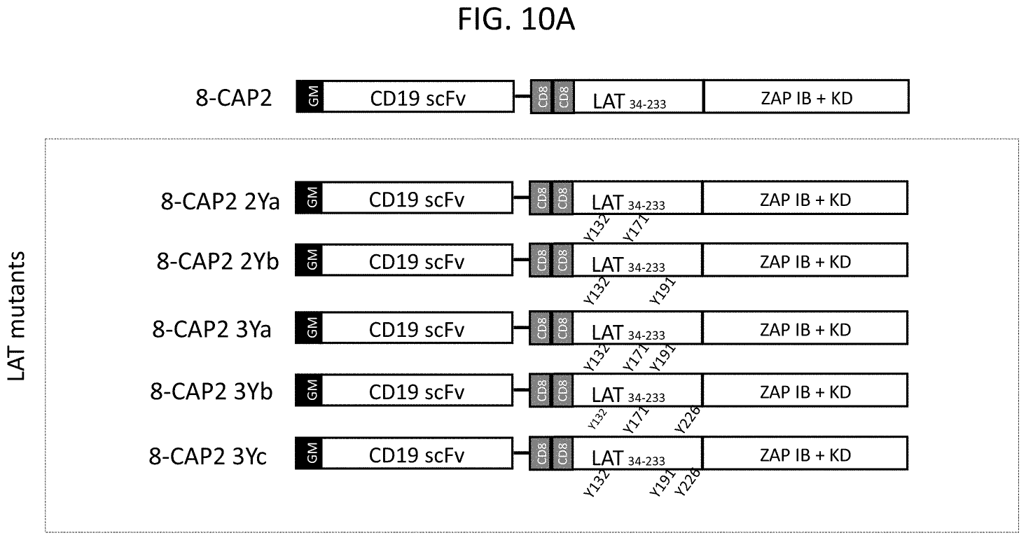

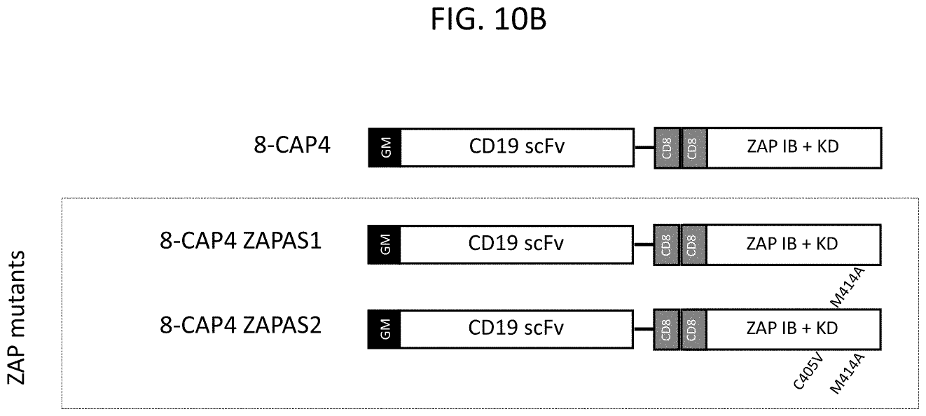

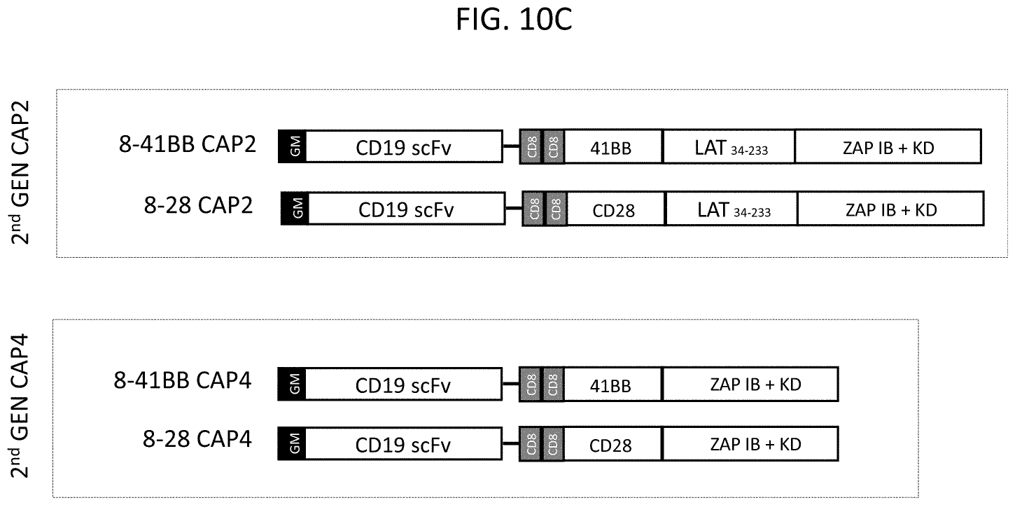

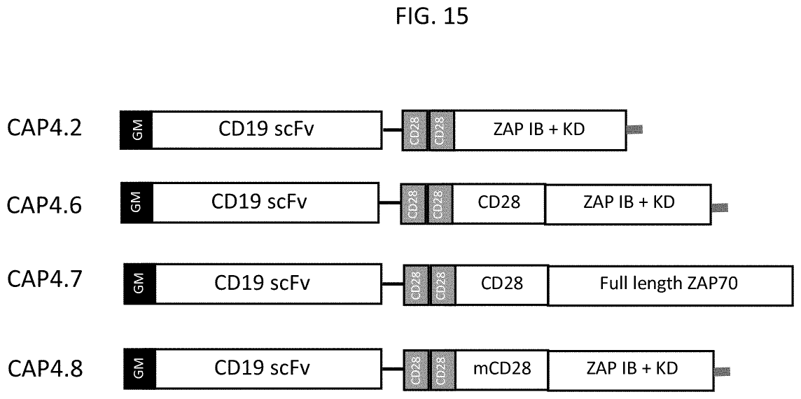

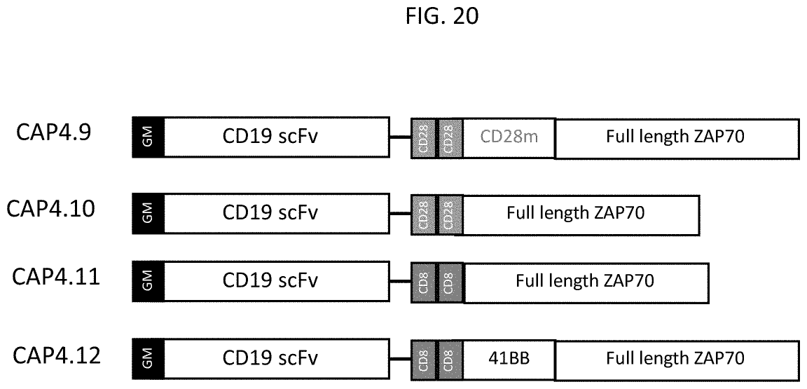

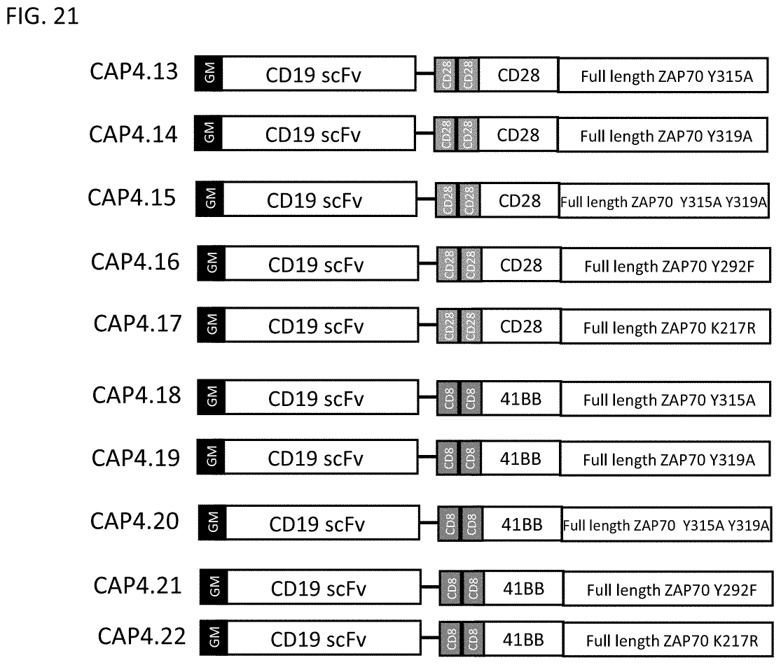

In some embodiments, the disclosed constructs include a full length ZAP70 protein. Without being bound by theory, inclusion of the full length ZAP70 protein may improve regulation of ZAP70 activity. In other embodiments, the disclosed constructs include a ZAP70 interdomain B (IB) domain and a ZAP70 KD or a ZAP70 KD. In any of the constructs described herein, such as the exemplary constructs illustrated with either a full length ZAP70 domain (e.g., FIGS. 15, 20, and 21) or a ZAP70 KD or ZAP70 IB and KD (e.g., FIGS. 1A, 5A, 6, 7, 10A-10C, and 15), any of the ZAP70 domains described herein can be used interchangeably.

In some examples, the ZAP70 domain is a full length ZAP70 protein. In some examples, the full length ZAP70 protein has an amino acid sequence at least 90%, at least 95%, at least 96%, at least 97%, at least 98%, or at least 99% identical to, or including or consisting of the amino acid sequence of amino acids 377-995 of SEQ ID NO: 55. In some examples, the full length ZAP70 is encoded by a nucleic acid at least 90%, at least 95%, at least 96%, at least 97%, at least 98%, or at least 99% identical to, or that includes or consists of nucleotides 1129-2985 of SEQ ID NO: 56.

In some examples, the ZAP70 domain is a ZAP70 kinase domain (KD). In other examples, the ZAP70 domain is a ZAP70 interdomain B (IB) domain and a ZAP70 KD. In some examples, the ZAP70 domain includes a ZAP70 KD, such as an amino acid sequence at least 90%, at least 95%, at least 96%, at least 97%, at least 98%, or at least 99% identical to, or including or consisting of the amino acid sequence of amino acids 527-789 of SEQ ID NO: 5. In some examples, the ZAP70 KD is encoded by a nucleic acid at least 90%, at least 95%, at least 96%, at least 97%, at least 98%, or at least 99% identical to, or includes or consists of nucleotides 1579-2367 of SEQ ID NO: 6.

In other examples the ZAP70 domain includes a ZAP70 IB, such as an amino acid sequence at least 90%, at least 95%, at least 96%, at least 97%, at least 98%, or at least 99% identical to, or including or consisting of the amino acid sequence of amino acids 522-604 of SEQ ID NO: 9 or amino acids 522-604 of SEQ ID NO: 100. In some examples, the ZAP70 IB is encoded by a nucleic acid at least 90%, at least 95%, at least 96%, at least 97%, at least 98%, or at least 99% identical to, or includes or consists of nucleotides 1564-1812 of SEQ ID NO: 10 or nucleotides 1564-1812 of SEQ ID NO: 101. In one non-limiting example, the CAP includes both a ZAP70 IB and a ZAP70 KD (e.g., a ZAP70 IB followed by a ZAP70 KD).

In additional embodiments, the ZAP70 domain includes one or more mutations. The analog sensitive (AS) ZAP70 mutants retain catalytic activity, but can be inhibited by a small molecule mutant-specific kinase inhibitor 3-MB-PP1 (see, e.g., Levin et al., J. Biol. Chem. 283:15419-15430, 2008). Incorporation of these mutations allow for mutant-specific, small molecule-mediated control of CAPs containing ZAP70 in a tunable and reversible way during therapy. In one example, the ZAP70 domain includes amino acid substitutions M414A and/or C405V, corresponding to amino acid positions M503 and C494 of SEQ ID NO: 27.

In other embodiments, the ZAP70 domain includes one or more mutations at a position corresponding to one or more of amino acids 217, 292, 315, and 319 of the full length ZAP70 protein (for example, corresponding to amino acids 593, 668, 691, and 695 of SEQ ID NO: 55). K217 in ZAP70 is targeted for ubiquitination, so mutation at this position may protect ZAP70 protein from degradation. Y292 in ZAP70 is the binding site for c-Cbl, a E3 ubiuitin ligase, so mutation at this amino acid may also protect ZAP70 protein from degradation. Y315 and Y319 are regulatory tyrosines in the Interdomain-B region of ZAP-70. Y315A and Y319A mutations have been shown to increase ZAP70 activity, so inclusion of these mutations could potentially increase CAP activity. In one example, the ZAP70 domain includes mutations at positions corresponding to amino acids 315 and 319 of the full length protein (e.g., corresponding to amino acids 691 and 695 of SEQ ID NO: 55). In particular examples, the mutations are selected from one or more of K217R, Y292F, Y315A, Y319A, and Y315A/Y319A.

E. Additional Components

In some embodiments, the disclosed CAPs include a linker between the LAT domain and the ZAP70 domain or between the transmembrane domain and ZAP70 domain, for example, to maintain flexibility between the domains of the CAP. In some examples, linker may include all or a portion of a ZAP70 protein, such as amino acids 318-333 of ZAP70. This sequence is part of the ZAP70 IB and can function to regulate the kinase activity of the ZAP70 KD. In some examples, the linker includes an amino acid sequence at least 90%, at least 95%, at least 96%, at least 97%, at least 98%, or at least 99% identical to, or including or consisting of the amino acid sequence of amino acids 504-519 of SEQ ID NO: 9. In other examples, the linker is encoded by a nucleic acid at least 90%, at least 95%, at least 96%, at least 97%, at least 98%, or at least 99% identical to, or includes or consists of nucleotides 1510-1557 of SEQ ID NO: 10. Additional linkers include glycine-serine peptide linkers, such as G₃S, G₄S, (G₃S)₃, (G₄S)₃, (G₄S)₄, (G₄S)₅, or (G₄S)₆. Other linkers can be selected as well.

In other embodiments, the disclosed CAPs include one or more additional intracellular signaling domains (for example from at least one of 41BB, CD28, ICOS, CD27, DAP10, and DAP12, or a combination of two or more thereof). In some examples, the additional intracellular signaling domain is C-terminal to the transmembrane domain and N-terminal to the LAT domain. In other examples, the CAP does not include a LAT domain and the additional intracellular signaling domain is C-terminal to the transmembrane domain and N-terminal to the ZAP70 domain. In some examples, the intracellular signaling domain is a 41BB intracellular signaling domain and includes an amino acid sequence at least 90%, at least 95%, at least 96%, at least 97%, at least 98%, or at least 99% identical to, or including or consisting of the amino acid sequence of amino acids 337-378 of SEQ ID NO: 43. In other examples, the intracellular signaling domain is a 41BB intracellular signaling domain and is encoded by a nucleic acid at least 90%, at least 95%, at least 96%, at least 97%, at least 98%, or at least 99% identical to, or includes or consists of nucleotides 1009-1134 of SEQ ID NO: 44.

In some examples, the intracellular signaling domain is a CD28 intracellular signaling domain and includes an amino acid sequence at least 90%, at least 95%, at least 96%, at least 97%, at least 98%, or at least 99% identical to, or including or consisting of the amino acid sequence of amino acids 337-377 of SEQ ID NO: 45. In other examples, the intracellular signaling domain is a CD28 intracellular signaling domain and is encoded by a nucleic acid at least 90%, at least 95%, at least 96%, at least 97%, at least 98%, or at least 99% identical to, or includes or consists of nucleotides 1009-1131 of SEQ ID NO: 46. In additional examples, the CD28 intracellular signaling domain includes one or more mutations. CD28 includes at least three intracellular subdomains (YMNM (SEQ ID NO: 67), PRRP (SEQ ID NO: 68), and PYAP (SEQ ID NO: 69)) that regulate costimulation upon TCR stimulation. CD28 directly activates PI3K and Grb2 signaling through YMNM (SEQ ID NO: 67), the PRRP (SEQ ID NO: 68) motif can associate with ITK, and the PYAP (SEQ ID NO: 69) subdomain initiates signaling by binding LCK. Thus, in some examples, the CD28 intracellular domain contains mutations in one or more or all of these three domains to decrease binding and thus signaling via these sites. In particular examples, the CD28 intracellular domain includes substitutions at one or more (such as 1, 2, 3, 4, or 5) amino acids corresponding to amino acid positions 347, 352, 355, 364, and 367 of SEQ ID NO: 53. In some examples, the substitutions include one or more or all of Y347F, P352A, P355A, P364A, and P367A. In one example, a mutated CD28 intracellular domain includes an amino acid sequence at least 90%, at least 95%, at least 96%, at least 97%, at least 98%, or at least 99% identical to, or including or consisting of the amino acid sequence of amino acids 336-376 of SEQ ID NO: 57 or is encoded by a nucleic acid at least 90%, at least 95%, at least 96%, at least 97%, at least 98%, or at least 99% identical to, or includes or consists of nucleotides 1006-1128 of SEQ ID NO: 58.

In some embodiments, the disclosed CAP constructs further include a domain that allows tracking of cells expressing the CAP. The domain is not part of the CAP, but may be expressed from the same nucleic acid as the CAP. In one example, the domain is a truncated EGFR polypeptide that does not include ligand binding or intracellular receptor tyrosine kinase activity, but retains cell surface localization and an epitope for anti-EGFR monoclonal antibodies (for example, cetuximab). An exemplary truncated EGFR polypeptide is described in Wang et al. (Blood 118:1255-1263, 2011) and use of the truncated EGFR polypeptide in the context of CARs is described in Li et al. (Cell Reports Medicine 2:100297, 2021).

F. Exemplary CAP Polypeptides and Nucleic Acids

Exemplary CAP polypeptides are provided herein, such as those shown schematically in FIGS. 1A, 5A, 6, 7, 10A-10C, 15, 20, and 21.

In some embodiments, the CAP includes (in N-terminal to C-terminal order) a GM-CSF signal sequence, an anti-CD19 scFv, a CD8 hinge domain, a CD8 transmembrane domain, a LAT 34-233 domain, and a ZAP70 IB and KD (referred to as 8-CAP2). In some examples, 8-CAP2 has an amino acid sequence at least 90%, at least 95%, at least 96%, at least 97%, at least 98%, or at least 99% identical to, or including or consisting of the amino acid sequence of SEQ ID NO: 25 or SEQ ID NO: 74 (8-CAP2-2). In some examples, 8-CAP2 is encoded by a nucleic acid molecule at least 90%, at least 95%, at least 96%, at least 97%, at least 98%, or at least 99% identical to, or including or consisting of the nucleic acid sequence of SEQ ID NO: 26 or SEQ ID NO: 75 (8-CAP2-2).

In other embodiments, the CAP includes (in N-terminal to C-terminal order) a GM-CSF signal sequence, an anti-CD19 scFv, a CD28 hinge domain, a CD28 transmembrane domain, a LAT 34-233 domain, and a ZAP70 KD (referred to as 28-CAP1). In some examples, 28-CAP1 has an amino acid sequence at least 90%, at least 95%, at least 96%, at least 97%, at least 98%, or at least 99% identical to, or including or consisting of the amino acid sequence of SEQ ID NO: 17. In some examples, 28-CAP1 is encoded by a nucleic acid molecule at least 90%, at least 95%, at least 96%, at least 97%, at least 98%, or at least 99% identical to, or including or consisting of the nucleic acid sequence of SEQ ID NO: 18.

In other embodiments, the CAP includes (in N-terminal to C-terminal order) a GM-CSF signal sequence, an anti-CD19 scFv, a CD28 hinge domain, a CD28 transmembrane domain, a LAT 34-233 domain, and a ZAP70 IB and KD (referred to as 28-CAP2). In some examples, 28-CAP2 has an amino acid sequence at least 90%, at least 95%, at least 96%, at least 97%, at least 98%, or at least 99% identical to, or including or consisting of the amino acid sequence of SEQ ID NO: 19 or SEQ ID NO: 70 (28-CAP2-2). In some examples, 28-CAP2 is encoded by a nucleic acid molecule at least 90%, at least 95%, at least 96%, at least 97%, at least 98%, or at least 99% identical to, or including or consisting of the nucleic acid sequence of SEQ ID NO: 20 or SEQ ID NO: 71 (28-CAP2-2).

In further embodiments, the CAP includes (in N-terminal to C-terminal order) a GM-CSF signal sequence, an anti-CD19 scFv, a CD8 hinge, a CD8 TM domain, a SLP-76 2-533 domain, and a ZAP70 KD (referred to as 8-SLP-CAP1). In other embodiments, the CAP includes (in N-terminal to C-terminal order) a GM-CSF signal sequence, an anti-CD19 scFv, a CD8 hinge, a CD8 TM domain, a SLP-76 2-533 domain, a ZAP70 IB, and a ZAP70 KD (referred to as 8-SLP-CAP2). In additional embodiments, the CAP includes (in N-terminal to C-terminal order) a GM-CSF signal sequence, an anti-CD19 scFv, a CD28 hinge, a CD28 TM domain, a SLP-76 2-533 domain, and a ZAP70 KD (referred to as 28-SLP-CAP1). In other embodiments, the CAP includes (in N-terminal to C-terminal order) a GM-CSF signal sequence, an anti-CD19 scFv, a CD28 hinge, a CD28 TM domain, a SLP-76 2-533 domain, a ZAP70 IB, and a ZAP70 KD (referred to as 28-SLP-CAP2). In further embodiments, the CAP includes (in N-terminal to C-terminal order) a GM-CSF signal sequence, an anti-CD19 scFv, a LAT extracellular and TM domain, a SLP-76 2-533 domain, a ZAP70 IB, and a ZAP70 KD (referred to as LAT-SLP-CAP2).

In other embodiments, the CAP includes (in N-terminal to C-terminal order) a GM-CSF signal sequence, an anti-CD19 scFv, a LAT extracellular and TM domain, a SLP-76 2-533 domain, a ZAP70 IB, and a ZAP70 KD (referred to as LAT-SLP-CAP1). In some examples, LAT-SLP76-CAP1 has an amino acid sequence at least 90%, at least 95%, at least 96%, at least 97%, at least 98%, or at least 99% identical to, or including or consisting of the amino acid sequence of SEQ ID NO: 15. In some examples, LAT-SLP76-CAP1 is encoded by a nucleic acid molecule at least 90%, at least 95%, at least 96%, at least 97%, at least 98%, or at least 99% identical to, or including or consisting of the nucleic acid sequence of SEQ ID NO: 16.

In other embodiments, the CAP includes (in N-terminal to C-terminal order) a GM-CSF signal sequence, an anti-CD19 scFv, a LAT hinge and TM domain, a SLP-76 2-533 domain, a ZAP70 IB, and a ZAP70 KD (referred to as LAT-CAP3). In some examples, LAT-CAP3 has an amino acid sequence at least 90%, at least 95%, at least 96%, at least 97%, at least 98%, or at least 99% identical to, or including or consisting of the amino acid sequence of SEQ ID NO: 21 or SEQ ID NO: 72 (LAT-CAP3-2). In some examples, LAT-CAP3 is encoded by a nucleic acid molecule at least 90%, at least 95%, at least 96%, at least 97%, at least 98%, or at least 99% identical to, or including or consisting of the nucleic acid sequence of SEQ ID NO: 22 or SEQ ID NO: 73 (LAT-CAP3-2).

In other embodiments, the CAP includes (in N-terminal to C-terminal order) a GM-CSF signal sequence, an anti-CD19 scFv, a CD28 hinge and TM domain, a ZAP70 IB, and a ZAP70 KD (referred to as 28-CAP4). In some examples, 28-CAP4 has an amino acid sequence at least 90%, at least 95%, at least 96%, at least 97%, at least 98%, or at least 99% identical to, or including or consisting of the amino acid sequence of SEQ ID NO: 23. In some examples, 28-CAP4 is encoded by a nucleic acid molecule at least 90%, at least 95%, at least 96%, at least 97%, at least 98%, or at least 99% identical to, or including or consisting of the nucleic acid sequence of SEQ ID NO: 24.

In other embodiments, the CAP includes (in N-terminal to C-terminal order) a GM-CSF signal sequence, an anti-CD19 scFv, a CD8 hinge domain, a CD8 transmembrane domain, a LAT 34-233 domain with tyrosines at positions Y132 and Y171, and a ZAP70 IB and KD (referred to as 8-CAP2 2Ya). In some examples, 8-CAP2 2Ya has an amino acid sequence at least 90%, at least 95%, at least 96%, at least 97%, at least 98%, or at least 99% identical to, or including or consisting of the amino acid sequence of SEQ ID NO: 29 or SEQ ID NO: 78 (8-CAP2 2Ya-2). In some examples, 8-CAP2 2Ya is encoded by a nucleic acid molecule at least 90%, at least 95%, at least 96%, at least 97%, at least 98%, or at least 99% identical to, or including or consisting of the nucleic acid sequence of SEQ ID NO: 30 or SEQ ID NO: 79 (8-CAP2 2Ya-2).

In additional embodiments, the CAP includes (in N-terminal to C-terminal order) a GM-CSF signal sequence, an anti-CD19 scFv, a CD8 hinge domain, a CD8 transmembrane domain, a LAT 34-233 domain with tyrosines at positions Y132 and Y191, and a ZAP70 IB and KD (referred to as 8-CAP2 2Yb). In some examples, 8-CAP2 2Yb has an amino acid sequence at least 90%, at least 95%, at least 96%, at least 97%, at least 98%, or at least 99% identical to, or including or consisting of the amino acid sequence of SEQ ID NO: 31 or SEQ ID NO: 80 (8-CAP2 2Yb-2). In some examples, 8-CAP2 2Yb is encoded by a nucleic acid molecule at least 90%, at least 95%, at least 96%, at least 97%, at least 98%, or at least 99% identical to, or including or consisting of the nucleic acid sequence of SEQ ID NO: 32 or SEQ ID NO: 81 (8-CAP2 2Yb-2).

In further embodiments, the CAP includes (in N-terminal to C-terminal order) a GM-CSF signal sequence, an anti-CD19 scFv, a CD8 hinge domain, a CD8 transmembrane domain, a LAT 34-233 domain with tyrosines at positions Y132, Y171, and Y191, and a ZAP70 IB and KD (referred to as 8-CAP2 3Ya). In some examples, 8-CAP2 3Ya has an amino acid sequence at least 90%, at least 95%, at least 96%, at least 97%, at least 98%, or at least 99% identical to, or including or consisting of the amino acid sequence of SEQ ID NO: 33 or SEQ ID NO: 82 (8-CAP2 3Ya-2). In some examples, 8-CAP2 3Ya is encoded by a nucleic acid molecule at least 90%, at least 95%, at least 96%, at least 97%, at least 98%, or at least 99% identical to, or including or consisting of the nucleic acid sequence of SEQ ID NO: 34 or SEQ ID NO: 83 (8-CAP2 3Ya-2).

In some embodiments, the CAP includes (in N-terminal to C-terminal order) a GM-CSF signal sequence, an anti-CD19 scFv, a CD8 hinge domain, a CD8 transmembrane domain, a LAT 34-233 domain with tyrosines at positions Y132, Y171, and Y226, and a ZAP70 IB and KD (referred to as 8-CAP2 3Yb). In some examples, 8-CAP2 3Yb has an amino acid sequence at least 90%, at least 95%, at least 96%, at least 97%, at least 98%, or at least 99% identical to, or including or consisting of the amino acid sequence of SEQ ID NO: 35 or SEQ ID NO: 84 (8-CAP2 3Yb-2). In some examples, 8-CAP2 3Yb is encoded by a nucleic acid molecule at least 90%, at least 95%, at least 96%, at least 97%, at least 98%, or at least 99% identical to, or including or consisting of the nucleic acid sequence of SEQ ID NO: 36 or SEQ ID NO: 85 (8-CAP2 3Yb-2).

In other embodiments, the CAP includes (in N-terminal to C-terminal order) a GM-CSF signal sequence, an anti-CD19 scFv, a CD8 hinge domain, a CD8 transmembrane domain, a LAT 34-233 domain with tyrosines at positions Y132, Y191, and Y226, and a ZAP70 IB and KD (referred to as 8-CAP2 3Yc). In some examples, 8-CAP2 3Yc has an amino acid sequence at least 90%, at least 95%, at least 96%, at least 97%, at least 98%, or at least 99% identical to, or including or consisting of the amino acid sequence of SEQ ID NO: 37 or SEQ ID NO: 86 (8-CAP2 3Yc-2). In some examples, 8-CAP2 3Yc is encoded by a nucleic acid molecule at least 90%, at least 95%, at least 96%, at least 97%, at least 98%, or at least 99% identical to, or including or consisting of the nucleic acid sequence of SEQ ID NO: 38 or SEQ ID NO: 87 (8-CAP2 3Yc-2).

In other embodiments, the CAP includes (in N-terminal to C-terminal order) a GM-CSF signal sequence, an anti-CD19 scFv, a CD8 hinge domain, a CD8 TM domain, a ZAP70 IB, and a ZAP70 KD (referred to as 8-CAP4). In some examples, 8-CAP4 has an amino acid sequence at least 90%, at least 95%, at least 96%, at least 97%, at least 98%, or at least 99% identical to, or including or consisting of the amino acid sequence of SEQ ID NO: 27 or SEQ ID NO: 76 (8-CAP4-2, also referred to as CAP4.3). In some examples, 8-CAP4 is encoded by a nucleic acid molecule at least 90%, at least 95%, at least 96%, at least 97%, at least 98%, or at least 99% identical to, or including or consisting of the nucleic acid sequence of SEQ ID NO: 28 or SEQ ID NO: 77 (8-CAP4-2). In some examples, 8-CAP4 further includes a M414A substitution in the ZAP70 domain (referred to as 8-CAP4 ZAPAS1).

In some examples, 8-CAP4 ZAPAS1 has an amino acid sequence at least 90%, at least 95%, at least 96%, at least 97%, at least 98%, or at least 99% identical to, or including or consisting of the amino acid sequence of SEQ ID NO: 39 or SEQ ID NO: 88 (8-CAP4 ZAPAS1-2). In some examples, 8-CAP4 ZAPAS1 is encoded by a nucleic acid molecule at least 90%, at least 95%, at least 96%, at least 97%, at least 98%, or at least 99% identical to, or including or consisting of the nucleic acid sequence of SEQ ID NO: 40 or SEQ ID NO: 89 (8-CAP4 ZAPAS1-2). In other examples, 8-CAP4 further includes a C405V and a M414A substitution in the ZAP70 domain (referred to as 8-CAP4 ZAPAS2). In some examples, 8-CAP4 ZAPAS2 has an amino acid sequence at least 90%, at least 95%, at least 96%, at least 97%, at least 98%, or at least 99% identical to, or including or consisting of the amino acid sequence of SEQ ID NO: 41 or SEQ ID NO: 90 (8-CAP4 ZAPAS2-2). In some examples, 8-CAP4 ZAPAS2 is encoded by a nucleic acid molecule at least 90%, at least 95%, at least 96%, at least 97%, at least 98%, or at least 99% identical to, or including or consisting of the nucleic acid sequence of SEQ ID NO: 42 or SEQ ID NO: 91 (8-CAP4 ZAPAS2-2).

In still further embodiments, the CAP includes (in N-terminal to C-terminal order) a GM-CSF signal sequence, an anti-CD19 scFv, a CD8 hinge domain, a CD8 transmembrane domain, a 41BB signaling domain, a LAT 34-233 domain, and a ZAP70 IB and KD (referred to as 8-41BB CAP2). In some examples, 8-41BB CAP2 has an amino acid sequence at least 90%, at least 95%, at least 96%, at least 97%, at least 98%, or at least 99% identical to, or including or consisting of the amino acid sequence of SEQ ID NO: 43 or SEQ ID NO: 92 (8-41BB CAP2-2). In some examples, 8-41BB CAP2 is encoded by a nucleic acid molecule at least 90%, at least 95%, at least 96%, at least 97%, at least 98%, or at least 99% identical to, or including or consisting of the nucleic acid sequence of SEQ ID NO: 44 or SEQ ID NO: 83 (8-41BB CAP2-2).

In additional embodiments, the CAP includes (in N-terminal to C-terminal order) a GM-CSF signal sequence, an anti-CD19 scFv, a CD8 hinge domain, a CD8 transmembrane domain, a CD28 signaling domain, a LAT 34-233 domain, and a ZAP70 IB and KD (referred to as 8-28 CAP2). In some examples, 8-28 CAP2 has an amino acid sequence at least 90%, at least 95%, at least 96%, at least 97%, at least 98%, or at least 99% identical to, or including or consisting of the amino acid sequence of SEQ ID NO: 45 or SEQ ID NO: 94 (8-28 CAP2-2). In some examples, 8-28 CAP2 is encoded by a nucleic acid molecule at least 90%, at least 95%, at least 96%, at least 97%, at least 98%, or at least 99% identical to, or including or consisting of the nucleic acid sequence of SEQ ID NO: 46 or SEQ ID NO: 95 (8-28 CAP2-2).

In other embodiments, the CAP includes (in N-terminal to C-terminal order) a GM-CSF signal sequence, an anti-CD19 scFv, a CD8 hinge domain, a CD8 transmembrane domain, a 41BB signaling domain, and a ZAP70 IB and KD (referred to as 8-41BB CAP4). In some examples, 8-41BB CAP4 has an amino acid sequence at least 90%, at least 95%, at least 96%, at least 97%, at least 98%, or at least 99% identical to, or including or consisting of the amino acid sequence of SEQ ID NO: 47 or SEQ ID NO: 96 (8-41BB CAP4-2). In some examples, 8-41BB CAP4 is encoded by a nucleic acid molecule at least 90%, at least 95%, at least 96%, at least 97%, at least 98%, or at least 99% identical to, or including or consisting of the nucleic acid sequence of SEQ ID NO: 48 or SEQ ID NO: 97 (8-41BB CAP4-2).

In additional embodiments, the CAP includes (in N-terminal to C-terminal order) a GM-CSF signal sequence, an anti-CD19 scFv, a CD8 hinge domain, a CD8 transmembrane domain, a CD28 signaling domain, and a ZAP70 IB and KD (referred to as 8-28 CAP4). In some examples, 8-28 CAP4 has an amino acid sequence at least 90%, at least 95%, at least 96%, at least 97%, at least 98%, or at least 99% identical to, or including or consisting of the amino acid sequence of SEQ ID NO: 49 or SEQ ID NO: 98 (8-28 CAP4-2). In some examples, 8-28 CAP4 is encoded by a nucleic acid molecule at least 90%, at least 95%, at least 96%, at least 97%, at least 98%, or at least 99% identical to, or including or consisting of the nucleic acid sequence of SEQ ID NO: 50 or SEQ ID NO: 99 (8-28 CAP4-2).

In additional embodiments, the CAP includes (in N-terminal to C-terminal order) a GM-CSF signal sequence, an anti-CD19 scFv, a CD28 hinge domain, a CD28 transmembrane domain, and a ZAP70 IB and KD (referred to as CAP4.2). In some examples, CAP4.2 has an amino acid sequence at least 90%, at least 95%, at least 96%, at least 97%, at least 98%, or at least 99% identical to, or including or consisting of the amino acid sequence of SEQ ID NO: 51. In some examples, CAP4.2 is encoded by a nucleic acid molecule at least 90%, at least 95%, at least 96%, at least 97%, at least 98%, or at least 99% identical to, or including or consisting of the nucleic acid sequence of SEQ ID NO: 52.

In another embodiment, the CAP includes (in N-terminal to C-terminal order) a GM-CSF signal sequence, an anti-CD19 scFv, a CD28 hinge domain, a CD28 transmembrane domain, a CD28 signaling domain, and a ZAP70 IB and KD (referred to as CAP4.6). In some examples, CAP4.6 has an amino acid sequence at least 90%, at least 95%, at least 96%, at least 97%, at least 98%, or at least 99% identical to, or including or consisting of the amino acid sequence of SEQ ID NO: 53. In some examples, CAP4.6 is encoded by a nucleic acid molecule at least 90%, at least 95%, at least 96%, at least 97%, at least 98%, or at least 99% identical to, or including or consisting of the nucleic acid sequence of SEQ ID NO: 54.

In a further embodiment, the CAP includes (in N-terminal to C-terminal order) a GM-CSF signal sequence, an anti-CD19 scFv, a CD28 hinge domain, a CD28 transmembrane domain, a CD28 signaling domain, and a full length ZAP70 domain (referred to as CAP4.7). In some examples, CAP4.7 has an amino acid sequence at least 90%, at least 95%, at least 96%, at least 97%, at least 98%, or at least 99% identical to, or including or consisting of the amino acid sequence of SEQ ID NO: 55. In some examples, CAP4.7 is encoded by a nucleic acid molecule at least 90%, at least 95%, at least 96%, at least 97%, at least 98%, or at least 99% identical to, or including or consisting of the nucleic acid sequence of SEQ ID NO: 56.

In other embodiments, the CAP includes (in N-terminal to C-terminal order) a GM-CSF signal sequence, an anti-CD19 scFv, a CD28 hinge domain, a CD28 transmembrane domain, a mutated CD28 signaling domain, and a ZAP70 IB and KD (referred to as CAP4.8). In some examples, CAP4.8 has an amino acid sequence at least 90%, at least 95%, at least 96%, at least 97%, at least 98%, or at least 99% identical to, or including or consisting of the amino acid sequence of SEQ ID NO: 57. In some examples, CAP4.8 is encoded by a nucleic acid molecule at least 90%, at least 95%, at least 96%, at least 97%, at least 98%, or at least 99% identical to, or including or consisting of the nucleic acid sequence of SEQ ID NO: 58.

In another embodiment, the CAP includes (in N-terminal to C-terminal order) a GM-CSF signal sequence, an anti-CD19 scFv, a CD28 hinge domain, a CD28 transmembrane domain, a mutated CD28 signaling domain, and a full length ZAP70 domain (referred to as CAP4.9). In some examples, CAP4.9 has an amino acid sequence at least 90%, at least 95%, at least 96%, at least 97%, at least 98%, or at least 99% identical to, or including or consisting of the amino acid sequence of SEQ ID NO: 59. In some examples, CAP4.9 is encoded by a nucleic acid molecule at least 90%, at least 95%, at least 96%, at least 97%, at least 98%, or at least 99% identical to, or including or consisting of the nucleic acid sequence of SEQ ID NO: 60.

In other embodiments, the CAP includes (in N-terminal to C-terminal order) a GM-CSF signal sequence, an anti-CD19 scFv, a CD28 hinge domain, a CD28 transmembrane domain, a linker, and a full length ZAP70 domain (referred to as CAP4.10). In some examples, CAP4.10 has an amino acid sequence at least 90%, at least 95%, at least 96%, at least 97%, at least 98%, or at least 99% identical to, or including or consisting of the amino acid sequence of SEQ ID NO: 61. In some examples, CAP4.10 is encoded by a nucleic acid molecule at least 90%, at least 95%, at least 96%, at least 97%, at least 98%, or at least 99% identical to, or including or consisting of the nucleic acid sequence of SEQ ID NO: 62.

In further embodiments, the CAP includes (in N-terminal to C-terminal order) a GM-CSF signal sequence, an anti-CD19 scFv, a CD8 hinge domain, a CD8 transmembrane domain, and a full length ZAP70 domain (referred to as CAP4.11). In some examples, CAP4.11 has an amino acid sequence at least 90%, at least 95%, at least 96%, at least 97%, at least 98%, or at least 99% identical to, or including or consisting of the amino acid sequence of SEQ ID NO: 63. In some examples, CAP4.11 is encoded by a nucleic acid molecule at least 90%, at least 95%, at least 96%, at least 97%, at least 98%, or at least 99% identical to, or including or consisting of the nucleic acid sequence of SEQ ID NO: 64.

In another embodiment, the CAP includes (in N-terminal to C-terminal order) a GM-CSF signal sequence, an anti-CD19 scFv, a CD8 hinge domain, a CD8 transmembrane domain, a 41BB signaling domain, and a full length ZAP70 domain (referred to as CAP4.12). In some examples, CAP4.12 has an amino acid sequence at least 90%, at least 95%, at least 96%, at least 97%, at least 98%, or at least 99% identical to, or including or consisting of the amino acid sequence of SEQ ID NO: 65. In some examples, CAP4.12 is encoded by a nucleic acid molecule at least 90%, at least 95%, at least 96%, at least 97%, at least 98%, or at least 99% identical to, or including or consisting of the nucleic acid sequence of SEQ ID NO: 66.

In further embodiments, the CAP includes (in N-terminal to C-terminal order) a GM-CSF signal sequence, an anti-CD19 scFv, a CD28 hinge, a CD28 TM domain, a CD28 intracellular signaling domain, and a full length ZAP70 domain with a Y315A substitution (referred to as CAP4.13). In other embodiments, the CAP includes (in N-terminal to C-terminal order) a GM-CSF signal sequence, an anti-CD19 scFv, a CD28 hinge, a CD28 TM domain, a CD28 intracellular signaling domain, and a full length ZAP70 domain with a Y319A substitution (referred to as CAP4.14). In additional embodiments, the CAP includes (in N-terminal to C-terminal order) a GM-CSF signal sequence, an anti-CD19 scFv, a CD28 hinge, a CD28 TM domain, a CD28 intracellular signaling domain, and a full length ZAP70 domain with Y315A and Y319A substitutions (referred to as CAP4.15). In other embodiments, the CAP includes (in N-terminal to C-terminal order) a GM-CSF signal sequence, an anti-CD19 scFv, a CD28 hinge, a CD28 TM domain, a CD28 intracellular signaling domain, and a full length ZAP70 domain with a Y292F substitution (referred to as CAP4.16). In further embodiments, the CAP includes (in N-terminal to C-terminal order) a GM-CSF signal sequence, an anti-CD19 scFv, a CD28 hinge, a CD28 TM domain, a CD28 intracellular signaling domain, and a full length ZAP70 domain with a K217R substitution (referred to as CAP4.17).

In further embodiments, the CAP includes (in N-terminal to C-terminal order) a GM-CSF signal sequence, an anti-CD19 scFv, a CD8 hinge, a CD8 TM domain, a 41BB intracellular signaling domain, and a full length ZAP70 domain with a Y315A substitution (referred to as CAP4.18). In other embodiments, the CAP includes (in N-terminal to C-terminal order) a GM-CSF signal sequence, an anti-CD19 scFv, a CD8 hinge, a CD8 TM domain, a 41BB intracellular signaling domain, and a full length ZAP70 domain with a Y319A substitution (referred to as CAP4.19). In additional embodiments, the CAP includes (in N-terminal to C-terminal order) a GM-CSF signal sequence, an anti-CD19 scFv, a CD8 hinge, a CD8 TM domain, a 41BB intracellular signaling domain, and a full length ZAP70 domain with Y315A and Y319A substitutions (referred to as CAP4.20). In other embodiments, the CAP includes (in N-terminal to C-terminal order) a GM-CSF signal sequence, an anti-CD19 scFv, a CD8 hinge, a CD8 TM domain, a 41BB intracellular signaling domain, and a full length ZAP70 domain with a Y292F substitution (referred to as CAP4.21). In further embodiments, the CAP includes (in N-terminal to C-terminal order) a GM-CSF signal sequence, an anti-CD19 scFv, a CD8 hinge, a CD8 TM domain, a 41BB intracellular signaling domain, and a full length ZAP70 domain with a K217R substitution (referred to as CAP4.22).

Also provided are functional variants of the CAPs or the domains thereof described herein, which retain the biological activity of the CAP of which it is a variant or retains the biological activity of the particular domain. The functional variant can be at least about 80%, about 85%, about 90%, about 91%, about 92%, about 93%, about 94%, about 95%, about 96%), about 97%, about 98%, about 99% or more identical in amino acid sequence to the parent CAP or domain. Substitutions can be made, for example, in one or more of the signal sequence domain, extracellular targeting domain, hinge domain, transmembrane domain, LAT domain, intracellular signaling domain, and ZAP70 domain.

In some examples, the functional variant includes the amino acid sequence of the parent CAP or domain with at least one conservative amino acid substitution (such as up to 10 conservative amino acid substitutions, for example, 1, 2, 3, 4, 5, 6, 7, 8, 9, or 10 conservative substitutions). In other examples, the functional variant includes the amino acid sequence of the parent CAP or domain with at least one non-conservative amino acid substitution (such as up to 10 non-conservative amino acid substitutions, for example, 1, 2, 3, 4, 5, 6, 7, 8, 9, or 10 non-conservative substitutions). In this case, the non-conservative amino acid substitution does not interfere with or inhibit the biological activity of the functional variant. The non-conservative amino acid substitution may enhance the biological activity of the functional variant, such that the biological activity of the functional variant is increased as compared to the parent CAP or domain.

The CAPs or the CAP domains can in some examples, include one or more synthetic amino acids in place of one or more naturally-occurring amino acids. Such synthetic amino acids include, for example, aminocyclohexane carboxylic acid, norleucine, a-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, oc-aminocycloheptane carboxylic acid, -(2-amino-2-norbornane)-carboxylic acid, γ-diaminobutyric acid, α,β-diaminopropionic acid, homophenylalanine, and α-tert-butylglycine. The CAPs may be glycosylated, amidated, carboxylated, phosphorylated, esterified, N-acylated, cyclized via, e.g., a disulfide bridge, or converted into an acid addition salt and/or optionally dimerized or polymerized, or conjugated.

### III. Cells Expressing Chimeric Adaptor Proteins

Also provided herein are cells (for example, immune cells) that express the disclosed CAPs and compositions including cells expressing the disclosed CAPs. In particular embodiments, the compositions include cells (such as T cells or natural killer (NK) cells) expressing a CAP and a pharmaceutically acceptable carrier.

In some embodiments, a nucleic acid molecule encoding a disclosed CAP is included in an expression vector (such as a viral vector) for expression in a host cell, such as a T cell or NK cell. In some examples, the expression vector includes a promoter operably linked to the nucleic acid molecule encoding the CAP. Additional expression control sequences, such as one or more enhancers, transcription and/or translation terminators, and initiation sequences can also be included in the expression vector.

The disclosed nucleic acids can be expressed in a host cell, such as a bacterial, plant, yeast, insect, or mammalian cell, for example, using an expression vector including a nucleic acid encoding the CAP. When the host is a eukaryote, methods of transfection of DNA such as calcium phosphate coprecipitation, microinjection, electroporation, insertion of a plasmid encased in liposomes, or virus vectors may be used. Eukaryotic cells can also be co-transformed with polynucleotide sequences encoding the CAP, and a second nucleic molecule encoding a selectable phenotype, such as the herpes simplex thymidine kinase gene. Another method is to use a eukaryotic viral vector, such as simian virus 40 (SV40), a lentivirus, or a retrovirus, to transduce or transform eukaryotic cells and express the CAP (see for example, Viral Expression Vectors, Springer Press, Muzyczka ed., 2011). In some examples, such expression systems are used to produce recombinant proteins in cells such as 293, COS, CHO, HeLa, or myeloma cell lines.

In some embodiments, a viral vector is utilized for expression of the CAP. Viral vectors include, but are not limited to simian virus 40, adenoviruses, adeno-associated virus (AAV), lentiviral vectors, and retroviruses, such as gamma retroviruses. Retroviral vectors provide a highly efficient method for gene transfer into eukaryotic cells. Moreover, retroviral integration takes place in a controlled fashion and results in the stable integration of one or a few copies of the new genetic information per cell. Without being bound by theory, lentiviral vectors have the advantage over vectors derived from onco-retroviruses such as murine leukemia viruses in that they can transduce non-proliferating cells, such as hepatocytes. They also have the added advantage of low immunogenicity. In one non-limiting example, the vector is a lentivirus vector such as pELNS, for example, with an EF1a promoter. Other exemplary vectors include pLV-ER1a-IRES-Neo, with neomycin deleted or retroviral vector MSGV.

Also provided are immune cells (such as T cells or NK cells) expressing a CAP disclosed herein. The immune cells are transduced or transformed with an expression vector including a nucleic acid encoding a CAP. In some examples, the transduced or transformed cells are peripheral blood lymphocytes (for example, obtained from a subject), peripheral blood mononuclear cells (for example, obtained from a subject), isolated T cells (such as a primary T cell or T cells obtained from a subject), or isolated NK cells (such as a primary NK cell or NK cells obtained from a subject). T cells or NK cells can be obtained from a sample from a subject, for example, blood, plasma, bone marrow, lymph node, or thymus. In some examples, T cells or NK cells are also enriched, purified, and/or expanded from a sample from a subject, for example before and/or after transduction or transformation with the CAP expression vector. In some examples, the T cells are a CD3⁺ T cell, such as a CD8⁺ T cell or a CD4⁺ T cell.

### IV. Methods of Immunotherapy

Disclosed herein are methods of treating cancer (such as a hematological malignancy or a solid tumor) in a subject, utilizing a CAP. In some embodiments, the methods include administering to the subject a composition including a T cell or NK cell expressing a CAP (for example, transduced with a vector encoding the CAP) and a pharmaceutically acceptable carrier. In other examples, the methods include administering to the subject a pharmaceutical composition including an expression vector encoding a CAP and a pharmaceutically acceptable carrier. The extracellular targeting domain of the CAP is selected based on the cancer being treated, for example, as shown in Table 1. In some examples, the subject is a human or veterinary subject.

Examples of hematological malignancies include leukemias, including acute leukemias (such as 11q23-positive acute leukemia, acute lymphocytic leukemia (ALL), T-cell ALL, acute myelocytic leukemia, acute myelogenous leukemia (AML), and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), lymphoblastic leukemia, polycythemia vera, lymphoma, diffuse large B cell lymphoma, Burkitt lymphoma, T cell lymphoma, follicular lymphoma, mantle cell lymphoma, Hodgkin disease, non-Hodgkin lymphoma, multiple myeloma, Waldenstrom macroglobulinemia, heavy chain disease, myelodysplastic syndrome, hairy cell leukemia, and myelodysplasia.

Examples of solid tumors include sarcomas (such as fibrosarcoma, myxosarcoma, liposarcoma, chondrosarcoma, osteogenic sarcoma, and other sarcomas), synovioma, mesothelioma, Ewing sarcoma, leiomyosarcoma, rhabdomyosarcoma, colon cancer, colorectal cancer, peritoneal cancer, esophageal cancer, pancreatic cancer, breast cancer (including basal breast carcinoma, ductal carcinoma and lobular breast carcinoma), lung cancer, ovarian cancer, prostate cancer, liver cancer (including hepatocellular carcinoma), gastric cancer, squamous cell carcinoma (including head and neck squamous cell carcinoma), basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, hepatoma, bile duct carcinoma, choriocarcinoma, Wilms tumor, cervical cancer, fallopian tube cancer, testicular tumor, seminoma, bladder cancer (such as renal cell cancer), melanoma, and CNS tumors (such as a glioma, glioblastoma, astrocytoma, medulloblastoma, craniopharyrgioma, ependymoma, pinealoma, hemangioblastoma, acoustic neuroma, oligodendroglioma, meningioma, neuroblastoma and retinoblastoma). Solid tumors also include tumor metastases (for example, metastases to the lung, liver, brain, or bone).

A variety of pharmaceutically acceptable carriers can be used in the compositions provided herein, for example, buffered saline and the like, for introducing the cells or vectors to a subject. These solutions are sterile and generally free of undesirable matter. The compositions may be sterilized. In some examples, the compositions also include pharmaceutically acceptable auxiliary substances such as pH adjusting and buffering agents, toxicity adjusting agents, and preservatives, for example, sodium acetate, sodium chloride, potassium chloride, calcium chloride, sodium lactate and the like. The concentration in these formulations can vary, and will be selected primarily based on fluid volumes, viscosities, body weight and the like in accordance with the particular mode of administration selected and the subject's needs.

The precise amount of the composition to be administered can be determined by a physician with consideration of individual differences in age, weight, tumor size, extent of metastasis, and condition of the patient (subject). In some embodiments, a pharmaceutical composition comprising the T cells or NK cells expressing a CAP described herein is administered at a dosage of about 10⁴ to 10⁹ cells/kg body weight (for example, about 10⁴, 10⁵, 10⁶, 10⁷, 10⁸, or 10⁹ cells/kg), such as about 10⁴ to 10⁶ cells/kg, about 10⁵ to 10⁷ cells/kg, or about 10⁶ to 10⁸ cells/kg. Exemplary doses are about 10⁵ cells/kg to about 10⁹ cells/kg, such as about 10⁶ cells/kg, about 5×10⁶ cells/kg, about 10⁷ cells/kg, about 5×10⁷ cells/kg, about 10⁸ cells/kg, or about 5×10⁸ cells/kg. The population of modified T cells or NK cells is typically administered parenterally, for example intravenously; however, injection or infusion to a tumor or close to a tumor (local administration) or administration to the peritoneal cavity can also be used. One of skill in the art can determine appropriate routes of administration.

In some examples, the composition (such as a composition including the T cells or NK cells expressing the CAP) is administered one or more times, such as 1, 2, 3, 4, 5, 6, 7, 8, 9, or 10 times. The composition can be administered by intravenous injection or infusion. In some examples, the composition is administered daily, weekly, bimonthly or monthly. If multiple doses are administered, the time between administrations may be 1, 2, 3, 4, 5, 6, 7, 8, 9, 10, 11, 12, 13, 14 days, or more. In some non-limiting examples, the composition is formulated for intravenous administration and is administered multiple times. The quantity and frequency of administration will be determined by such factors as the condition of the subject, and the type and severity of the subject's disease, although appropriate dosages may be determined by clinical trials.

In some examples, the CAP-modified T cells or NK cells are able to persist and/or replicate in vivo in the subject, resulting in long-term persistence that can lead to sustained tumor control. In same examples, the T or NK cells cells administered to the subject, or the progeny of these cells, persist in the subject for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, or for years after administration to the subject. In other embodiments, the cells or their progeny are present for less than six months, five month, four months, three months two months, or one month, e.g., three weeks, two weeks, one week, after administration of the cells to the subject.

In one embodiment, the CAP is introduced into cells, such T cells or NK cells, and the subject receives an administration of the cells. In some embodiments, the methods include isolating T or NK cells from a subject, transducing or transforming the T or NK cells with an expression vector (such as a lentiviral vector or a retroviral vector) encoding the CAP to produce modified T cells or NK cells, and administering the modified T cells or NK cells expressing the CAP to the subject with cancer for treatment. The T cells or NK cells can be autologous to a recipient or allogeneic (for example, the isolated and transformed T cells or NK cells are not from the subject being treated). In some examples, the subject may undergo an immunosuppressive regimen (e.g., lymphodepletion or partial lymphodepletion) prior to administering the modified T cells or NK cells. Immune system supportive therapies (such as IL-2 and/or G-CSF) may also be administered to the subject, for example to promote expansion of the modified cells in the subject and/or to support recovery of neutrophils.

In some embodiments, a population of cells including lymphocytes (such as PBMCs) can be obtained by any method, including, but not limited to apheresis. All or a portion of the population of cells can be utilized immediately or all or a portion of the cells can be cryopreserved for future use. When ready for use, all or a portion of the population of cells is thawed (if previously cryopreserved) and T cells or NK cells are activated, enriched, and/or expanded in culture. Methods of isolating, activating, and expanding T cells or NK cells are known in the art (e.g., WO 2018/006054 and WO 2018/022646, incorporated herein by reference in their entirety). The cells are transduced or transformed with a vector including a CAP. In particular examples, about 10⁷-10⁹ cells are transduced or transformed (for example, about 1×10⁷, 5×10⁷, 1×10⁸, 5×10⁸, or 1×10⁹ cells). Transduced or transformed T cells or NK cells can be expanded ex vivo and can be cryopreserved at appropriate dosage amounts (for example, about 10⁶ to 10¹² cells) following expansion. The transduced or transformed T cells or NK cells are thawed (if previously frozen), prior to administration to the subject. The subject may undergo an immunosuppressive regimen (e.g., lymphodepletion) prior to administering the modified T cells or NK cells. The modified T cells or NK cells are administered to the subject, for example by infusion.

Treatment efficacy is monitored by methods such as tumor size, number of lesions, tumor stage, response rate, or other criteria. In some examples, a decrease in size of a primary tumor or metastases (for example, as defined by standard RECIST or irRECIST criteria) indicates inhibition of cancer in the subject. In other examples, progression-free survival and/or overall survival (for example, for 1 month, 3 months, 6 months, 9 months, 12 months, 18 months 2 years, or more, such as 1-12 months, 6-18 months, 1-2 years, or more) indicates inhibition of cancer in the subject. In other examples, one or more of persistence of circulating CAP-expressing T cells or NK cells, changes in immune cell subsets and activation status of T cells, as well as other immunologic determinants are evaluated, with clinical outcomes evaluated at baseline (e.g., prior to or at the time of administration of the modified cells), at different time points during treatment, and at the time of disease progression.

In some examples, the subject is also treated with one or more of surgery, chemotherapy, radiation, immunosuppressive agents, and chemotherapeutic agents. Exemplary agents include, but are not limited to alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and BBR3464), busulfan, dacarbazine, mechlorethamine, procarbazine, temozolomide, thiotepa, and uramustine; antimetabolites, such as folic acid (for example, methotrexate, pemetrexed, and raltitrexed), purine (for example, cladribine, clofarabine, fludarabine, mercaptopurine, and thioguanine), pyrimidine (for example, capecitabine), cytarabine, fluorouracil, and gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, and valrubicin), bleomycin, hydroxyurea, and mitomycin; topoisomerase inhibitors, such as topotecan and irinotecan; monoclonal antibodies, such as alemtuzumab, bevacizumab, cetuximab, gemtuzumab, rituximab, panitumumab, atezolizumab, avelumab, ipilimumab, ofatumumab, nivolumab, pembrolizumab, rituximab, durvalumab, and trastuzumab; photosensitizers, such as aminolevulinic acid, methyl aminolevulinate, porfimer sodium, and verteporfin; proteasome inhibitors, such as bortezomib, carfilzomib, oprozomib, ixazomib, marizomib, and delanzomib; kinase inhibitors, such as gefitinib, imatinib, sunitinib, sorafenib, vemurafenib, trametinib, and ruxolitinib; growth factor receptor inhibitors, such as acitinib, erlotinib, cabozantinib, and crizotinib; mTOR inhibitors, such as everolimus, temsirolimus, and temisorotimus; and other agents, such as alitretinoin, altretamine, amsacrine, anagrelide, arsenic trioxide, asparaginase, bexarotene, celecoxib, denileukin diftitox, enzalutamide, flutamide, nilutamide, bicalutamide, topilutamide, apalutamide, estramustine, hydroxycarbamide, pentostatin, masoprocol, mitotane, pegaspargase, tamoxifen, clomifene, raloxifene, anastrozole, fulvestrant, and tretinoin. Additional agents include checkpoint inhibitors, such as antibodies (e.g., nivolumab, pembrolizumab, ipilimumab, durvalumab, and atezolizumab) or small molecule inhibitors (e.g., BMS-1001, BMS-1166, CCX4503).

In some embodiments, the subject can also be administered an agent which enhances the activity of a CAP-expressing cell. For example, in one embodiment, the agent can inhibit a molecule that decreases the ability of a CAP-expressing cell to mount an immune effector response. Examples of such inhibitory molecules include PD-1, PD-L1, CTLA4, TIM3, LAG3, VISTA, BTLA, TIGIT, LAIR1, CD160, 2B4 and TGFR-beta. In one embodiment, the inhibitor of an inhibitory signal is an antibody or antibody fragment that binds to an inhibitory molecule. For example, the agent can be an antibody or antibody fragment that binds to PD-1 (e.g., pembrolizumab, nivolumab, pidilizumab, or lambrolizumab), PD-L1 (e.g., MDPL3280A, YW243.55.S70, or MDX-1 105), PD-L2 (e.g., Amplimmune), or CTLA4 (e.g., ipilimumab or tremelimumab), TM3, or LAG3. In other examples, the inhibitor is a small molecule checkpoint inhibitor, such as BMS-1001, BMS-1166, BMS936559, CCX4503, MAX10043, MAX10129.

### EXAMPLES

The following examples are provided to illustrate certain particular features and/or embodiments. These examples should not be construed to limit the disclosure to the particular features or embodiments described.

### Example 1

### Materials and Methods

DNA constructs: The generation and use of the following constructs have been previously described in Yi et al., Nat. Commun. 10:277, 2019): GRB2-Emerald, TCRζ-Halo, ZAP70-Halo, SLP76-Halo, Grb2-Halo and PLCyl-Halo. 28z CAR (Kochenderfer et al., J. Immunother. 32:689-702, 2009) and CD4-LAT (Balagopalan et al., J. Immunol. 190:3849-3853, 2013) have been previously described. Using Infusion cloning methods, GFP was first added to CD4-LAT to generate CD4-LAT-GFP. In a second step of infusion cloning, ZAP70 KD was inserted between CD4-LAT and GFP with a 20aa linker between LAT and ZAP70KD to generate CD4-CAP-GFP. GFP was then removed from CD4-CAP-GFP by restriction digest to generate CD4-CAP. CD19-CAP was generated by swapping out the CD4 signal sequence and extracellular domain for GM-CSF signal sequence and anti-CD19 scFv.

Reagents: Human anti-CD3 (clone HIT3a), anti-CD4 (clone OKT4), anti-CD45 and anti-CD43 monoclonal antibodies were purchased from BD Pharmingen and were used to coat coverslips for imaging assays. The following antibodies were used for immunostaining: mouse monoclonal anti-TCRζ (pY142) antibody (BD Biosciences), mouse monoclonal anti-phosphotyrosine (clone 4G10, EMD Millipore). Halo-tag ligand conjugated to Janelia Fluor (JF-646) was from Janelia Research Institute.

Cell culture and transient transfection of Jurkat cells: Jurkat E6.1 cells have been described previously (Balagopalan et al., Mol. Cell Biol. 27:8622-8636, 2007). E6.1 Jurkat cells were cultured in RPMI (Life Technologies), 10% fetal bovine serum (Life Technologies), and 1% penicillin-streptomycin (Life Technologies). For transient transfections, 1×10⁶ Jurkat cells were transfected with 2 μg DNA using the Nucleofector Kit V (Lonza, catalog no. VCA-1003), Program X-001 24 hours prior to imaging. Prior to imaging, Jurkat cells were spun down and resuspended in imaging buffer (20 mM Hepes pH 7.2, 137 mM NaCl, 5 mM KCl, 0.7 mM Na₂HPO₄, 6 mM D-glucose, 2 mM MgCl₂, 1 mM CaCl₂), 1% BSA).

Generation of K562CD19 cell line: The cDNA for full-length CD19 was purchased from Invitrogen and cloned into the MSGV retroviral backbone. Retrovirus-containing supernatant was prepared as described below under transient retrovirus and lentivirus production. K562 cells (ATCC) were transduced with this supernatant and then CD19-expressing cells were sorted by flow cytometry to obtain a population of K562 cells that uniformly expressed high levels of CD19.

Transient retrovirus and lentivirus production: To transiently produce retrovirus (CD19) and lentivirus (28z CAR and CAP), 293T cells were transfected with the CAR or CAP expressing viral vector along with packaging plasmid psPAX2 (Addgene) and a plasmid encoding viral envelope protein pVSV-G (Addgene) using Lipofectamine 2000 (Invitrogen). The transfected cells were incubated at 37° C. for 6-8 hours in DMEM medium without antibiotics (DMEM, 10% heat-inactivated FBS). The medium used for transfection was then replaced with fresh DMEM medium (DMEM, 10% heat-inactivated FBS, 1% Penn-Strep) and the cells were incubated for another 36-48 hours. Supernatant containing retro- or lentiviruses was removed from the dishes and centrifuged to remove cellular debris and used for transduction experiments.

Cell culture and lentiviral transduction of primary human T cells: Primary human peripheral blood T cells were obtained without donor identifiers from the National Institutes of Health Blood Bank. Mononuclear cells were isolated by Ficoll density gradient centrifugation. Isolated cells were resuspended at a concentration of 1×10⁶ cell per mL in T cell medium containing 50 ng/mL OKT3 and 300 IU/mL of IL-2 (Day 0). Cells were cultured in 75 cm² flasks were cultured upright at 37° C. and 5% C02. On Day 2 of stimulation, cells were transduced using lentiviral supernatants transiently produced by 293T cells. Transductions were performed by spinoculation of cells for 2 hours at 2000×g at 28° C. on retronectin coated plates. Lentiviral transduction was repeated on Day 3. Cells were cultured at 37° C. in 5% CO₂ for 6 days in exponential growth phase. On Day 6, expression of CD3, CD4, CD8 and CD19 were assessed by flow cytometry as described below.

Live cell imaging: Preparation of antibody-coated coverslip has been described previously (Bunnell et al., Sci. STKE 2003:PL8, 2003). Jurkat cells were added to HIT3a or OKT4 antibody-coated 8-well coverslip chambers (Lab-Tek, Thermo Fisher) and imaged in imaging buffer. Jurkat cells expressing Halo-tagged constructs were labeled with 100 nM Janelia Fluor Halo-647 ligand for 30 minutes at 37° C. Cells were imaged after three washes in complete medium and then resuspended in imaging buffer. All chambers used for live cell imaging contained imaging buffer. TIRF images from live cells were collected with a Nikon Ti-E inverted microscope, using a 100×SR Apochromat TIRF objective lens (1.49 numerical aperture), and an Andor iXon Ultra 897 EM charge-coupled device camera (512×512 pixels, 16 m pixel). Time-lapse images were collected at 3 sec/frame

Immunofluorescence imaging: Jurkat T cells were allowed to adhere to the HIT3a antibody-coated coverslip for 2 min at 37° C. in imaging buffer and then fixed for 30 min in 4% (wt/vol) PFA solution (Electron Microscopy Sciences). Samples were permeabilized in 0.1% Triton-X-100 for 3 minutes and then incubated in a blocking solution consisting of 10% FBS (Sigma-Aldrich), 0.01% sodium azide (Sigma-Aldrich), 1×PBS for 1 h at room temperature (RT). After three washes in 1×PBS, the cells were stained with anti-phosphotyrosine or anti-pTCR antibody in blocking solution (20 μg/ml) for 1 hour at RT. The cells were washed 3× in PBS and imaged using the TIRF microscope as detailed above.

Flow cytometry: Cell surface markers on primary human PBMCs were evaluated by flow cytometry on Day 6 after isolation and stimulation with OKT3 and IL-2 as described above. Cells were stained with anti-CD4-APC, anti-CD8-FITC, anti-CD3-PerCP and anti-CD19-PE (eBiosciences) to evaluate percentages and profiles of T cells. For CAR or CAP detection, biotin-labeled polyclonal goat anti-mouse-F(ab)₂ antibodies (anti-Fab, Jackson Immunoresearch) were added to detect the CD19 scFv. Biotin-labeled normal polyclonal goat IgG antibodies (Jackson Immunoresearch) served as an isotype control. The cells were incubated at 4° C. for 25 minutes and washed once. The cells were suspended in FACs buffer (1×PBS, 10% FBS, 0.02% sodium azide) and blocked with normal mouse IgG (Invitrogen). The cells were then stained with phycoerythrin (PE)-labeled streptavidin (BD Pharmingen). Flow cytometry acquisition was performed on a BD LSRFortessa Cell Analyzer (BD Biosciences), and analysis was performed with FlowJo (Treestar, Inc.).

IL-2 and IFN-γ Enzyme-Linked Immunosorbent Assay (ELISA): Target K562 or K562CD19 cells were washed and suspended at 1×10⁶ cells per mL in T cell media without IL-2. 1×10⁵ target cells of each target cell type were added to the wells of a 96 well round bottom plate (Corning). Effector T cell cultures were washed and suspended at 1×10⁶ cells per mL in T cell media without IL-2. 1×10⁵ effector T cells were combined with 1×10⁵ target cells in a total volume of 200 μl in the wells of the round bottom 96 well plate. In addition, wells containing T cells alone were prepared. As a positive control T cells were stimulated with plate-bound anti-CD3 (10 μg/ml) and anti-CD28 (1 μg/ml). The plates were incubated at 37° C. for 18-20 hours. Following the incubation, supernatants were harvested and then subjected to IL-2 or IFNγ ELISA assay as per manufacturer's instructions (R&D Systems).

Luciferase based killing assay: Transduced or untransduced PBMCs were co-cultured with NALM6 or NALM6 CD19 knockout tumor cells stably expressing luciferase at a 5:1 or 1:1 ratio as indicated. After 4 hours, luminescence was detected by adding Steady Glo reagent (Promega). Luminescence from target only wells (max CPS) and target only wells plus 1% Tween-20 (min CPS) was used to determine assay range. Percent specific lysis was calculated as: 1-(sample CPS-min CPS)/(max CPS-min CPS).

### Example 2

### Characterization of LAT-Based CD4 and CD19 Chimeric Adapter Proteins

LAT-based chimeric adapter proteins (CAP) were constructed (FIG. 1A). E6.1 Jurkat T cells expressing TCRζ-Emerald were activated on anti-CD3 antibody (HIT3)-coated coverslips. E6.1 Jurkat T cells expressing CD4-LAT and GRB2-Emerald or CD4-CAP-GFP were activated on anti-CD4 antibody (OKT4)-coated coverslips and structures defined as microclusters were observed when cells were activated. Microclusters were not present in anti-CD4-activated CD4-LAT-expressing cells (FIG. 1C) but were observed in CD4-CAP-GFP-expressing cells (FIG. 1D), suggesting that a chimeric molecule containing LAT and ZAP70-KD can be used to activate T cell signaling.

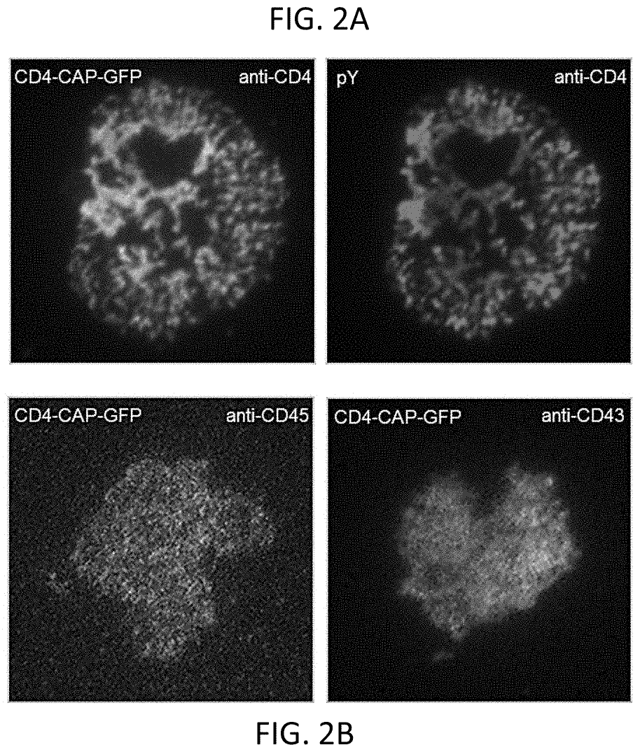

E6.1 Jurkat T cells expressing CD4-CAP-GFP were activated on anti-CD4 antibody (OKT4)-coated coverslip, fixed and stained with anti-phospho-Tyrosine (pY) antibody (4G10). The pY signal co-localized with the CD4-CAP-GFP signal showing that these microclusters are sites of active signaling (FIG. 2A). CD4-CAP-expressing cells did not form microclusters on anti-CD45 or anti-CD43 antibody-coated coverslips (FIG. 2B) showing that CD4-CAP-GFP activation is specific to CD4 ligation.

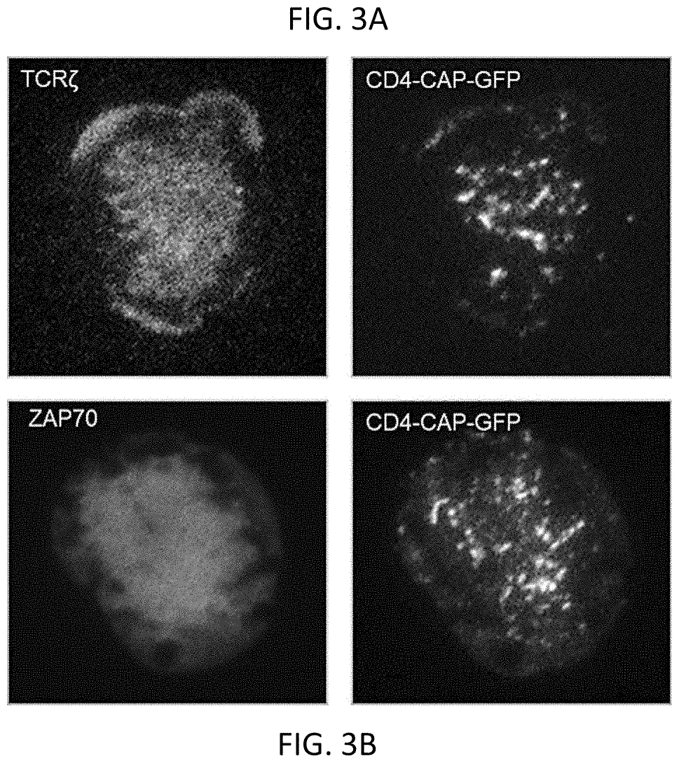

E6.1 Jurkat T cells expressing TCRζ-Halo and CD4-CAP-GFP or ZAP70-Halo and CD4-CAP-GFP were activated on anti-CD4 antibody (OKT4)-coated coverslips. TCRζ-Halo (FIG. 3A) and ZAP70-Halo (FIG. 3B) did not localize to CD4-CAP-GFP microclusters, showing that activation of CAP is independent of the TCR complex. TCRζ and ZAP-70 are normally recruited to microclusters downstream of anti-CD3 stimulation, as shown in FIG. 1.

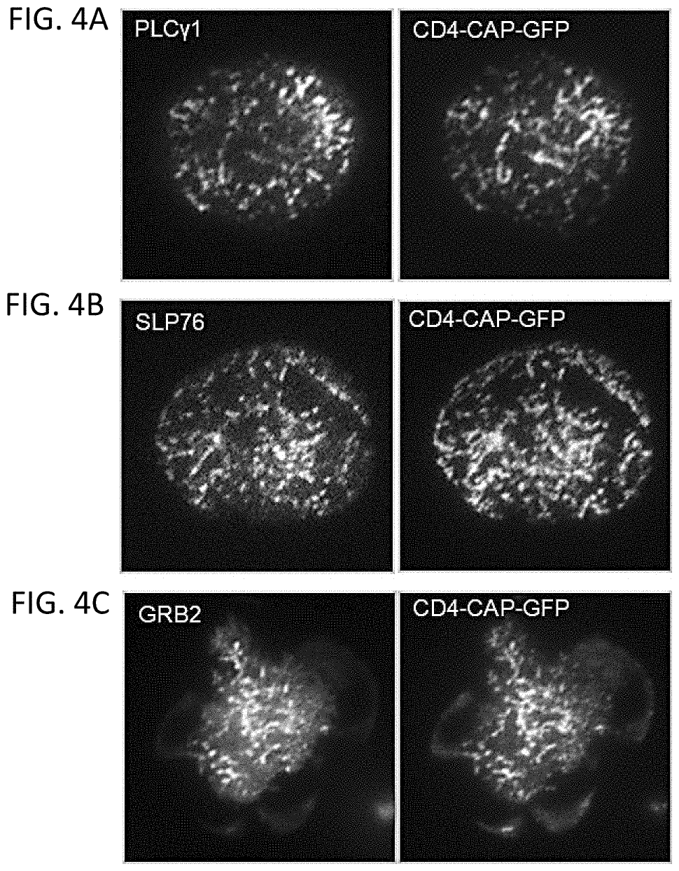

E6.1 Jurkat T cells expressing CD4-CAP-GFP and PLCγ1-Halo (FIG. 4A), SLP76-Halo (FIG. 4B), or GRB2-Halo (FIG. 4C) were activated on anti-CD4 antibody (OKT4)-coated coverslips. CD4-CAP-GFP recruited to microclusters upon anti-CD4 stimulation. PLCγ1-Halo, SLP76-Halo and GRB2-Halo signaling proteins were also localized to CD4-CAP-GFP microclusters. This demonstrates that LAT-associated signaling complexes are formed in CAP activated T cells.

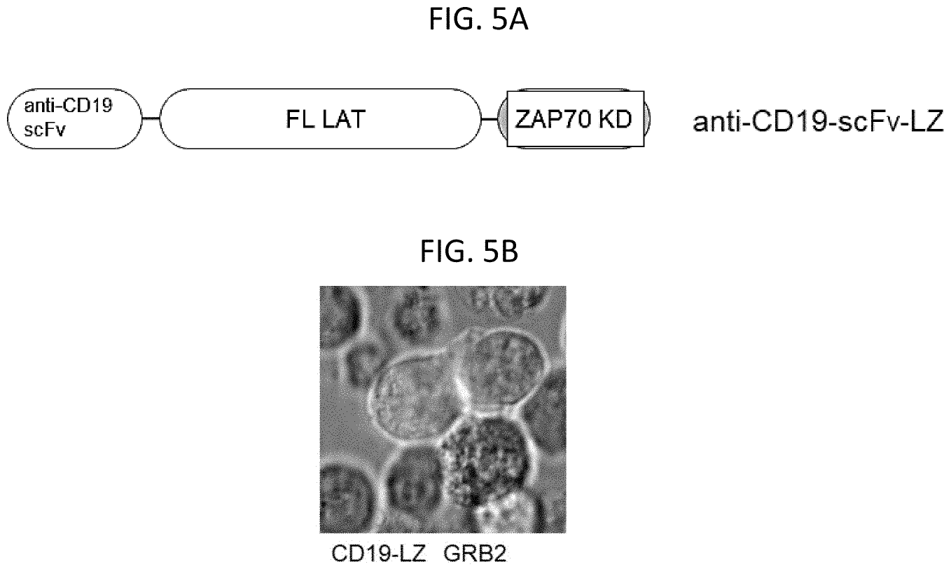

An anti-CD19-scFv-LAT-ZAP70 KD construct was also produced (FIG. 5A). A CD19-LZ-GFP and GRB2-Scarlet-expressing E6.1 cell formed a conjugate with a Raji B cell (FIG. 5B). Microclusters containing CD19-LZ-GFP and GRB2-Scarlet were formed near the center of the conjugate, showing T cell activation.

### Example 3

### Further Evaluation of CD19-CAP Molecules

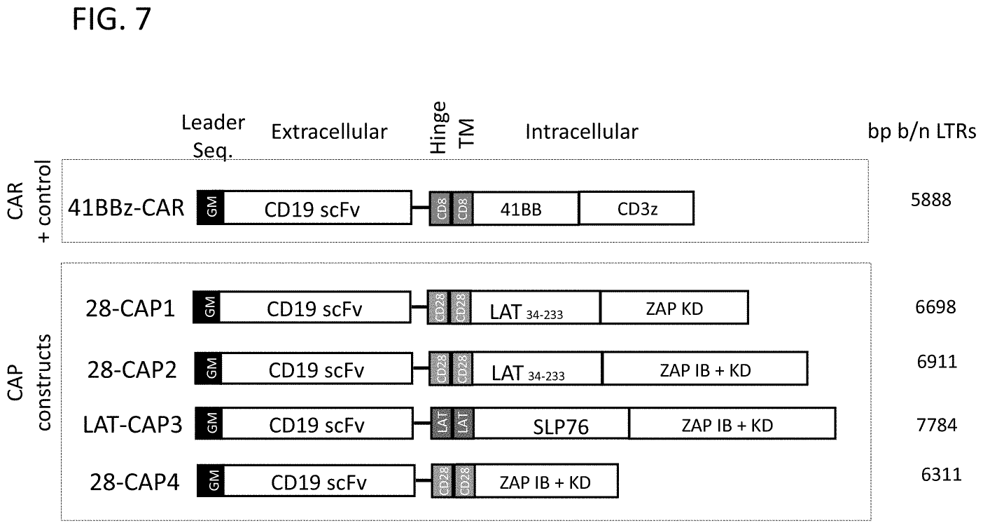

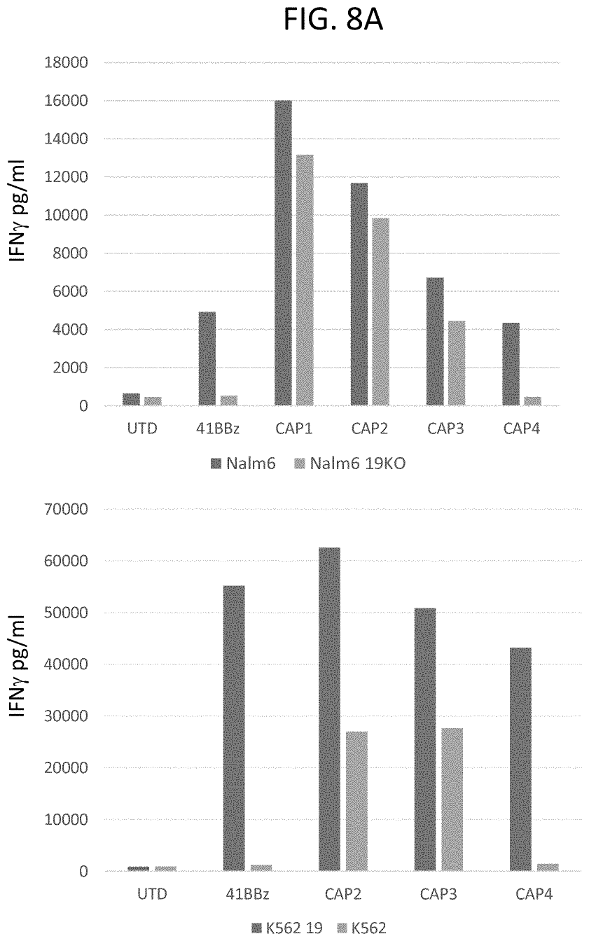

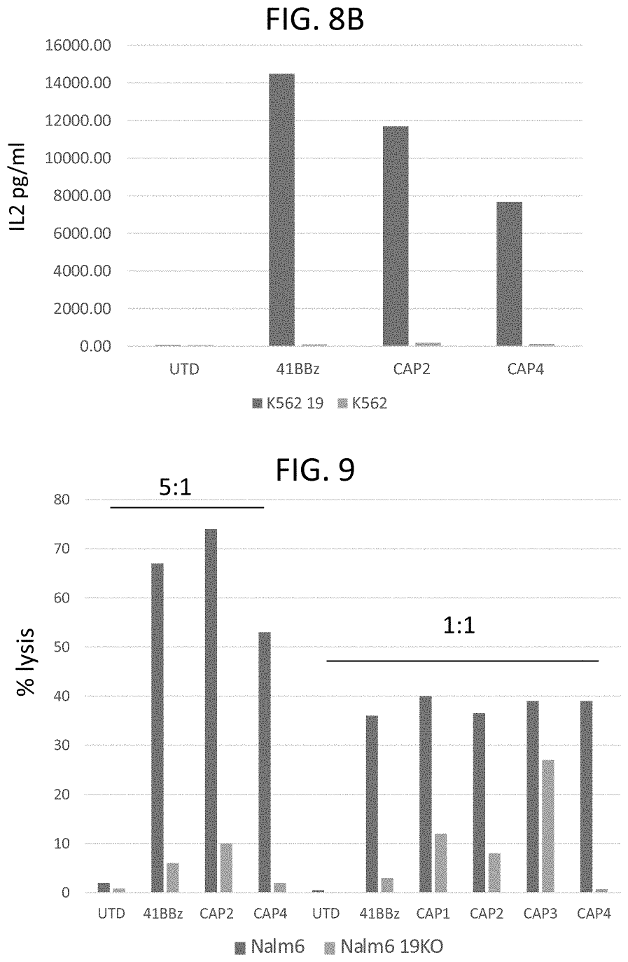

PBMCs transduced with CAP1, CAP2 and CAP3 (FIG. 7) released high amounts of interferon 7 upon co-culture with Nalm6 and K652 CD19 cells at a 1:1 ratio (FIG. 8A), but also displayed high levels of antigen-independent cytokine release in Nalm6 CD19 KO and K562 cells not expressing CD19. In comparison, CAP4 expressing PBMCs showed low basal, but high antigen-induced interferon 7 release, similar to the 41BBz control. PBMCs transduced with CAP2 and CAP4 released interleukin-2 upon co-culture with K562 CD19 expressing cells, demonstrating functional activation (FIG. 8B). CAP1, CAP2, CAP3 and CAP4 expressing PBMCs showed similar cytolytic activity towards Nalm6 CD19⁺ acute lymphoblastic leukemia cells as 41BBz CAR positive control at 5:1 and 1:1 effector:target ratios (FIG. 9).

### Example 4

### In Vivo Evaluation of CAP Efficacy

This example describes methods that can be used to evaluate efficacy of one or more of the disclosed CAPs in vivo in a mouse xenograft model. However, one skilled in the art will appreciate that methods that deviate from these specific methods can also be used to test efficacy of CAPs in vivo.

NSG mice are injected intravenously or subcutaneously with luciferase-expressing human lymphoma or leukemia cell lines (including NAML6 leukemia cell line), followed by treatment with human T cells genetically modified to express CAP constructs. Mock-transduced T cells (such as constructs that are designed not to work or have specificity toward irrelevant target) are used as negative controls. One or more CARs that are proven to work against target malignancies are used as a positive control. Leukemia burden is detected using the Xenogen IVIS Lumina (Caliper Life Sciences).

### Example 5

### Treating Leukemia with CD19-CAP Expressing T Cells

This example describes methods that can be used to treat a subject with leukemia with CD19-CAP transduced T cells. However, one skilled in the art will appreciate that methods that deviate from these specific methods can also be used to successfully treat a subject with leukemia. In addition, similar methods can be used to treat a subject with other CAP-expressing T cells and other cancers.

A subject with leukemia (such as acute lymphoblastic leukemia) undergoes apheresis to collect peripheral blood mononuclear cells. T cells are activated and transduced with a retroviral vector including a CD19-CAP, such as those described herein (e.g., SEQ ID NOs: 5-10 and 15-50). The transduced T cells are expanded. The subject undergoes chemotherapy-induced lymphodepletion 2-14 days prior to infusion with the transduced T cells.

### Example 6

### Treating B-Cell Lymphoma with CD19-CAP Expressing NK Cells

This example describes methods that can be used to treat a subject with a B-cell lymphoma with CD19-CAP transduced NK cells. However, one skilled in the art will appreciate that methods that deviate from these specific methods can also be used to successfully treat a subject with B-cell lymphoma. In addition, similar methods can be used to treat a subject with other CAP-expressing NK cells and other cancers.

A subject with a B-cell lymphoma undergoes apheresis to collect peripheral blood mononuclear cells. NK cells (e.g., CD56-positive/CD3-negative cells) are isolated from the PBMCs by positive and/or negative selection using immune-magnetic methods. transduced with a retroviral vector including a CD19-CAP, such as those described herein (e.g., SEQ ID NOs: 5-10 and 15-50). The transduced NK cells can be cryopreserved for later use or can be formulated for administration to the subject (for example, in a pharmaceutically acceptable carrier). A composition comprising 10⁶ to 10¹² of the expanded NK cells is administered to the subject intravenously.

### Example 7

### Additional In Vitro Evaluation of CAP4 Constructs

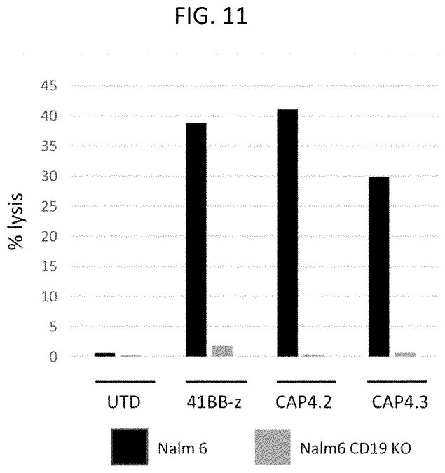

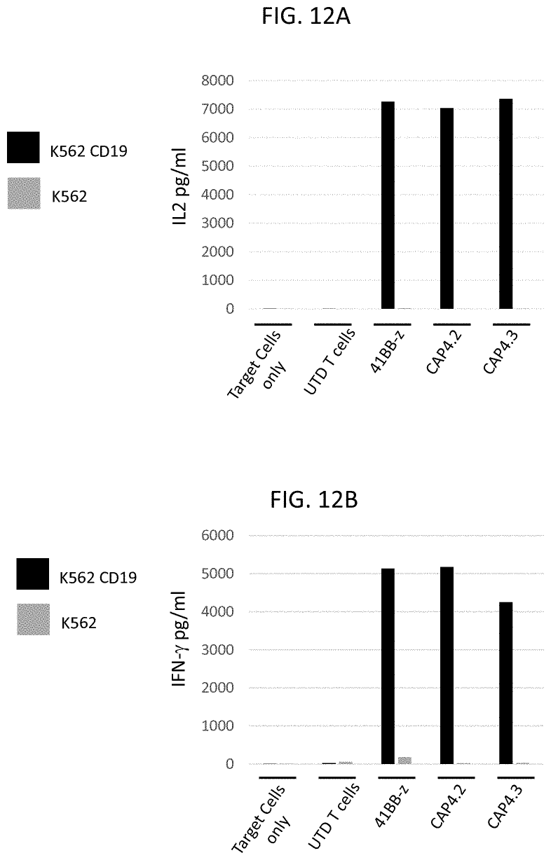

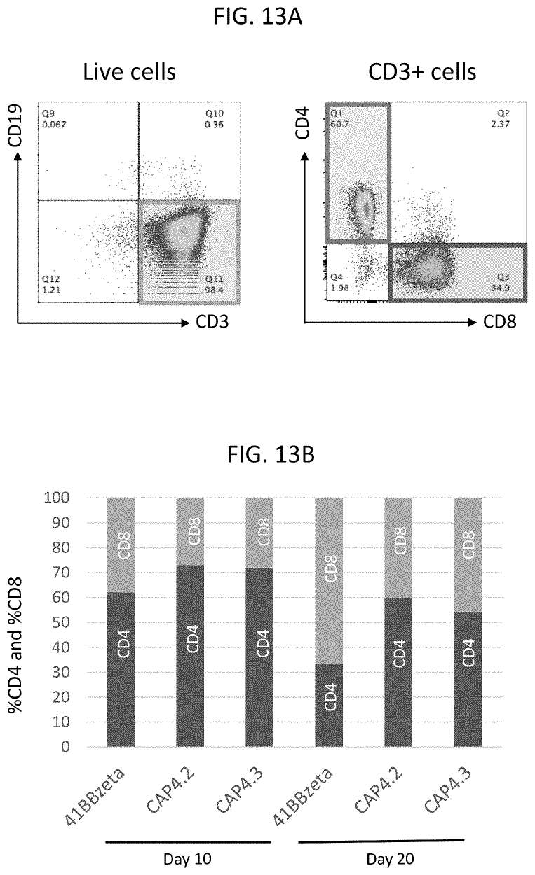

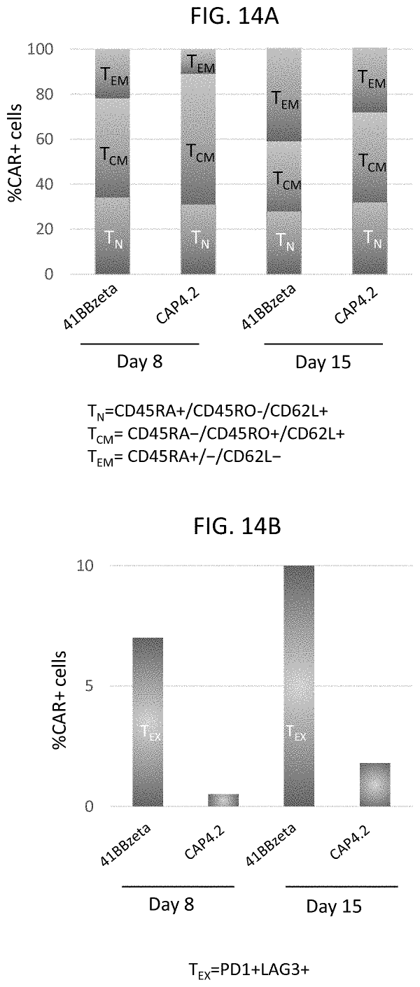

Cytolytic killing of Nalm6 CD19⁺ and Nalm6 CD19 knockout cells by T cells expressing CAP4.2 (SEQ ID NO: 51) or CAP4.3 (also referred to as 8-CAP4-2; SEQ ID NO: 76) was evaluated. As shown in FIG. 11, both CAPs selectively killed CD19-expressing cells. The CAP-expressing T cells also selectively elicited cytokine production by K562 cells expressing CD19 (FIGS. 12A and 12B). Primary human T cells transduced with CAR and CAP constructs were gated on CD3+ cells (FIG. 13A) and evaluated for CD4 and CD8 populations on Day 10 and 20 in culture (FIG. 13B). The basal T cell activation state of CAP4.2 T cells was evaluated. The CAP4.2 T cells were basally less activated (FIG. 14A) and less exhausted (FIG. 14B) than 41BBzeta CAR T cells.

### Example 8

### In Vitro and In Vivo Evaluation of Additional CAP4 Construct

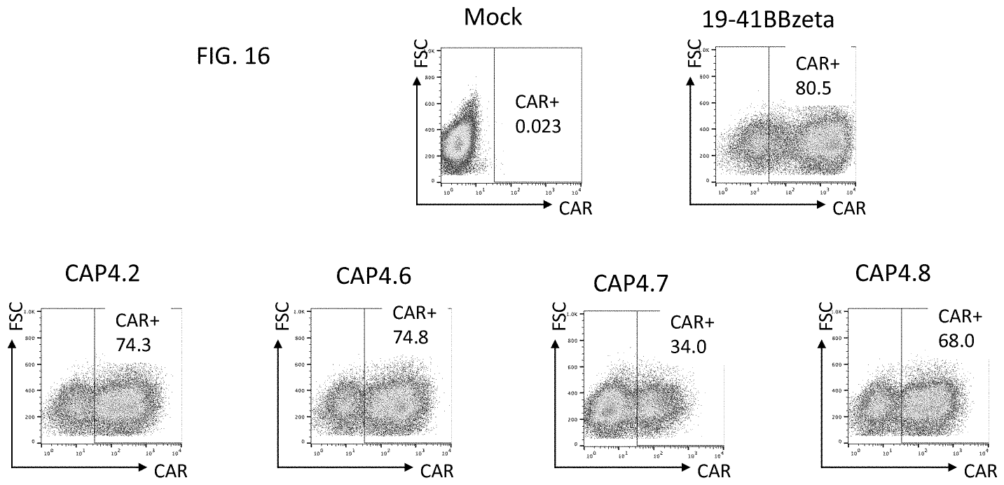

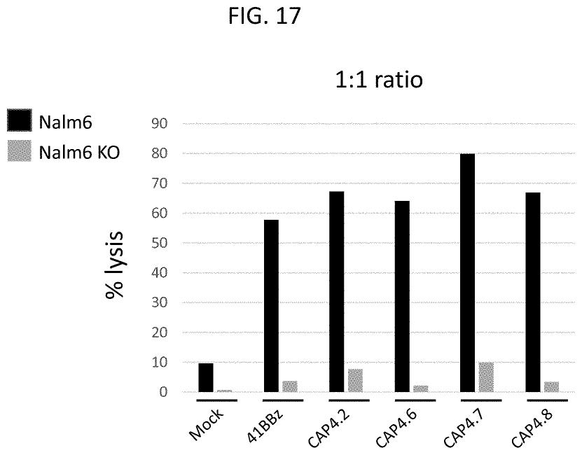

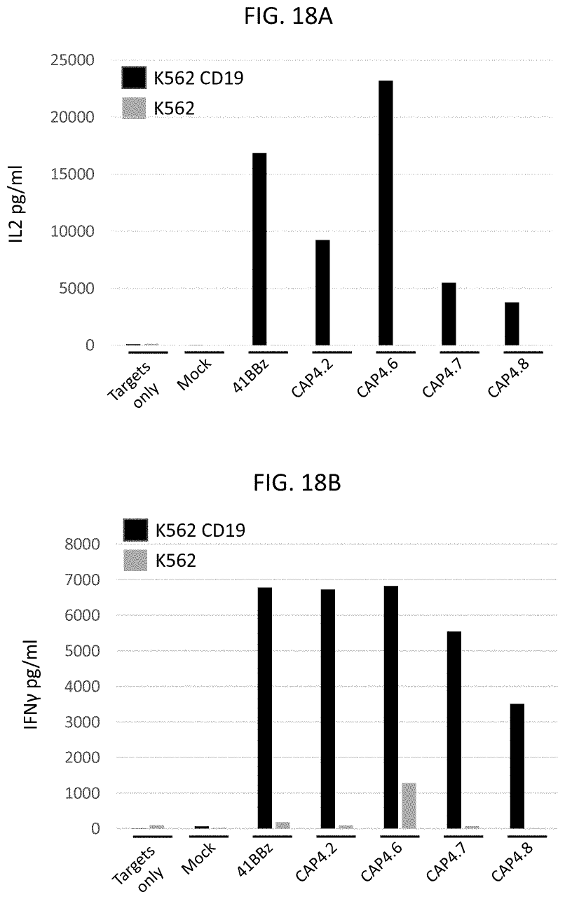

A series of additional CAP4 constructs were generated (FIG. 15). Expression of the constructs in T cells was evaluated (FIG. 16). As shown in FIG. 17, each CAP selectively killed CD19-expressing cells. The CAP-expressing T cells also selectively elicited cytokine production by K562 cells expressing CD19 (FIGS. 18A and 18B).

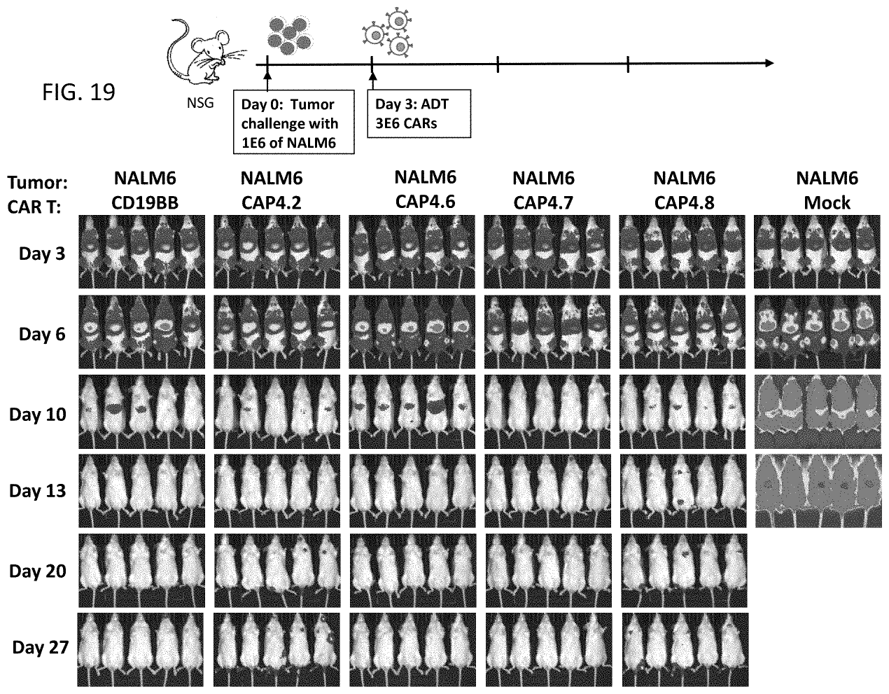

NSG mice were injected intravenously with 1×10⁶ luciferase-expressing NAML6 leukemia cell line, followed by treatment on day 3 with human 3×10⁶ T cells genetically modified to express CAP constructs (FIG. 19). Mock-transduced T cells were used as negative controls. CD19BBzeta CAR was used as a positive control. Leukemia burden was detected using the Xenogen IVIS Lumina (Caliper Life Sciences). As shown in FIG. 19, the CAP constructs were effective at reducing or even eliminating tumor burden at day 27.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A chimeric polypeptide comprising:
(a) an extracellular targeting domain;
(b) a transmembrane domain; and
(c) a ZAP70 domain, wherein (a)-(c) are in N-terminal to C-terminal order.

2. The chimeric polypeptide of claim 1, further comprising:
a hinge domain, wherein the hinge domain is C-terminal of the extracellular targeting domain and N-terminal of the transmembrane domain;
a signal sequence domain, wherein the signal sequence is N-terminal of the extracellular targeting domain;
an intracellular signaling domain selected from a 41BB intracellular signaling domain and a CD28 intracellular signaling domain, wherein the intracellular signaling domain is C terminal of the transmembrane domain and N-terminal of the ZAP70 domain; or
any combination thereof.

3. The chimeric polypeptide of claim 1, wherein the ZAP70 domain comprises a full length ZAP70 kinase, a ZAP70 kinase domain, or a ZAP70 interdomain B and a ZAP70 kinase domain.

4. The chimeric polypeptide of claim 3, wherein the full length ZAP70 kinase comprises the amino acid sequence of amino acids 377-995 of SEQ ID NO: 55, the ZAP70 kinase domain comprises the amino acid sequence of amino acids 527-789 of SEQ ID NO: 5, or the ZAP70 interdomain B comprises the amino acid sequence of amino acids 522-604 of SEQ ID NO: 9 or amino acids 522-604 of SEQ ID NO: 100.

5. The chimeric polypeptide of claim 3, wherein the ZAP70 domain comprises an amino acid substitution at an amino acid position corresponding to M503 of SEQ ID NO: 27, C494 of SEQ ID NO: 27, K593 of SEQ ID NO: 55, Y668 of SEQ ID NO: 55, Y691 of SEQ ID NO: 55, Y695 of SEQ ID NO: 55, or a combination of two or more thereof.

6. The chimeric polypeptide of claim 2, wherein the hinge domain is a CD8 hinge domain or a CD28 hinge domain.

7. The chimeric polypeptide of claim 6, wherein the hinge domain comprises the amino acid sequence of amino acids 270-308 of SEQ ID NO: 17, amino acids 266-312 of SEQ ID NO: 25, or EEA.

8. The chimeric polypeptide of claim 2, wherein the signal sequence domain is a human granulocyte-macrophage colony-stimulating factor (GM-CSF) signal sequence.

9. The chimeric polypeptide of claim 1, wherein the extracellular targeting domain comprises an antigen binding domain or scFv that binds to a target protein of interest.

10. The chimeric polypeptide of claim 9, wherein the target protein of interest is a tumor associated antigen.

11. The chimeric polypeptide of claim 10, wherein the extracellular targeting domain binds to CD19.

12. The chimeric polypeptide of claim 11, wherein the extracellular targeting domain binds to CD19 and comprises the amino acid sequence of amino acids 23-267 of SEQ ID NO: 17.

13. The chimeric polypeptide of claim 1, wherein the transmembrane domain is a CD8 transmembrane domain, a CD28 transmembrane domain, or a LAT transmembrane domain.

14. The chimeric polypeptide of claim 13, wherein the transmembrane domain comprises the amino acid sequence of amino acids 313-336 of SEQ ID NO: 25, amino acids 309-335 of SEQ ID NO: 17, or amino acids 271-295 of SEQ ID NO: 15.

15. The chimeric polypeptide of claim 2, wherein the 41BB intracellular domain comprises the amino acid sequence of amino acids 337-378 of SEQ ID NO: 43 or wherein the CD28 intracellular domain comprises the amino acid sequence of amino acids 337-377 of SEQ ID NO: 45 or the amino acid sequence of amino acids 336-376 of SEQ ID NO: 57.

16. The chimeric polypeptide of claim 1, further comprising:
an intracellular linker for activation of T cells (LAT) domain or an SLP-76 domain, wherein the LAT domain or the SLP-76 domain is C-terminal of the transmembrane domain and N-terminal of the ZAP70 domain.

17. The chimeric polypeptide of claim 1, comprising the amino acid sequence of any one of SEQ ID NOs: 23, 27, 39, 41, 47, 49, 51, 53, 55, 57, 59, 61, 63, 65, 76, 88, 90, 96, and 98.

18. An isolated nucleic acid molecule encoding the chimeric polypeptide of claim 1.

19. The isolated nucleic acid of claim 18, comprising the nucleic acid sequence of any one of SEQ ID NOs: 24, 28, 40, 42, 48, 50, 52, 54, 56, 58, 60, 62, 64, 66, 77, 89, 91, 97, and 99.

20. An expression vector comprising the nucleic acid molecule of claim 18.

21. A T cell or natural killer (NK) cell transduced with the vector of claim 20 or a composition comprising the transduced T cell or NK cell and a pharmaceutically acceptable carrier.

22. A method of treating a subject with cancer, comprising administering to the subject an effective amount of the cell or composition of claim 21.

23. The method of claim 22, wherein the T cell or NK cell is autologous to the subject.

24. The method of claim 22, wherein the cancer is a hematological malignancy or a solid tumor.


SEQUENCE LISTING (101 sequences)

SEQ ID NO: 1 - protein, 624 aa, Artificial Sequence
  CD4-LAT
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 2 - DNA, 1872 bp, Artificial Sequence
  CD4 LAT
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtcgaccgaggaggccatcctggtcccctgcgtgctggggctcctgctgctgcccatcctggccatgttgatggcactgtgtgtgcactgccacagactgccaggctcctacgacagcacatcctcagatagtttgtatccaaggggcatccagttcaaacggcctcacacggttgccccctggccacctgcctacccacctgtcacctcctacccacccctgagccagccagacctgctccccatcccaagatccccgcagccccttgggggctcccaccggacgccatcttcccggcgggattctgatggtgccaacagtgtggcgagctacgagaacgaggaaccagcctgtgaggatgcagatgaggatgaggacgactatcacaacccaggctacctggtggtgcttcctgacagcaccccggccactagcactgctgccccatcagctcctgcactcagcacccctggcatccgagacagtgccttctccatggagtccattgatgattacgtgaacgttccggagagcggggagagcgcagaagcgtctctggatggcagccgggagtatgtgaatgtgtcccaggaactgcatcctggagcggctaagactgagcctgccgccctgagttcccaggaggcagaggaagtggaggaagagggggctccagattacgagaatctgcaggagctgaac

SEQ ID NO: 3 - protein, 911 aa, Artificial Sequence
  CD4-CAP
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASK

SEQ ID NO: 4 - DNA, 2733 bp, Artificial Sequence
  CD4-CAP
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaacttccccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagcttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtcgaccgaggaggccatcctggtcccctgcgtgctggggctcctgctgctgcccatcctggccatgttgatggcactgtgtgtgcactgccacagactgccaggctcctacgacagcacatcctcagatagtttgtatccaaggggcatccagttcaaacggcctcacacggttgccccctggccacctgcctacccacctgtcacctcctacccacccctgagccagccagacctgctccccatcccaagatccccgcagccccttgggggctcccaccggacgccatcttcccggcgggattctgatggtgccaacagtgtggcgagctacgagaacgaggaaccagcctgtgaggatgcagatgaggatgaggacgactatcacaacccaggctacctggtggtgcttcctgacagcaccccggccactagcactgctgccccatcagctcctgcactcagcacccctggcatccgagacagtgccttctccatggagtccattgatgattacgtgaacgttccggagagcggggagagcgcagaagcgtctctggatggcagccgggagtatgtgaatgtgtcccaggaactgcatcctggagcggctaagactgagcctgccgccctgagttcccaggaggcagaggaagtggaggaagagggggctccagattacgagaatctgcaggagctgaacggaccctacagcgacccagaggagctcaaggacaagaagctcttcctgaagcgcgataacctcctcatagctgacattgaacttggctgcggcaactttggctcagtgcgccagggcgtgtaccgcatgcgcaagaagcagatcgacgtggccatcaaggtgctgaagcagggcacggagaaggcagacacggaagagatgatgcgcgaggcgcagatcatgcaccagctggacaacccctacatcgtgcggctcattggcgtctgccaggccgaggccctcatgctggtcatggagatggctgggggcgggccgctgcacaagttcctggtcggcaagagggaggagatccctgtgagcaatgtggccgagctgctgcaccaggtgtccatggggatgaagtacctggaggagaagaactttgtgcaccgtgacctggcggcccgcaacgtcctgctggttaaccggcactacgccaagatcagcgactttggcctctccaaagcactgggtgccgacgacagctactacactgcccgctcagcagggaagtggccgctcaagtggtacgcacccgaatgcatcaacttccgcaagttctccagccgcagcgatgtctggagctatggggtcaccatgtgggaggccttgtcctacggccagaagccctacaagaagatgaaagggccggaggtcatggccttcatcgagcagggcaagcggatggagtgcccaccagagtgtccacccgaactgtacgcactcatgagtgactgctggatctacaagtgggaggatcgccccgacttcctgaccgtggagcagcgcatgcgagcctgttactacagcctggccagcaag

SEQ ID NO: 5 - protein, 789 aa, Artificial Sequence
  anti-CD19-scFv-LZ
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSTEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGPYSDPEELKDKKLFLKSRLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 6 - DNA, 2367 bp, Artificial Sequence
  anti-CD19-scFv-LZ
atggcgctccctgtcaccgcactgcttcttccgctggcactgctgctgcacgctgcacggcctgagcaaaaacttatctctgaagaggacctcgatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttcatcgaccgaggaggccatcctggtcccctgcgtgctggggctcctgctgctgcccatcctggccatgttgatggcactgtgtgtgcactgccacagactgccaggctcctacgacagcacatcctcagatagtttgtatccaaggggcatccagttcaaacggcctcacacggttgccccctggccacctgcctacccacctgtcacctcctacccacccctgagccagccagacctgctccccatcccaagatccccgcagccccttgggggctcccaccggacgccatcttcccggcgggattctgatggtgccaacagtgtggcgagctacgagaacgaggaaccagcctgtgaggatgcagatgaggatgaggacgactatcacaacccaggctacctggtggtgcttcctgacagcaccccggccactagcactgctgccccatcagctcctgcactcagcacccctggcatccgagacagtgccttctccatggagtccattgatgattacgtgaacgttccggagagcggggagagcgcagaagcgtctctggatggcagccgggagtatgtgaatgtgtcccaggaactgcatcctggagcggctaagactgagcctgccgccctgagttcccaggaggcagaggaagtggaggaagagggggctccagattacgagaatctgcaggagctgaacggaccctacagcgacccagaggagctcaaggacaagaagctcttcctgaagtctagactcatagctgacattgaacttggctgcggcaactttggctcagtgcgccagggcgtgtaccgcatgcgcaagaagcagatcgacgtggccatcaaggtgctgaagcagggcacggagaaggcagacacggaagagatgatgcgcgaggcgcagatcatgcaccagctggacaacccctacatcgtgcggctcattggcgtctgccaggccgaggccctcatgctggtcatggagatggctgggggcgggccgctgcacaagttcctggtcggcaagagggaggagatccctgtgagcaatgtggccgagctgctgcaccaggtgtccatggggatgaagtacctggaggagaagaactttgtgcaccgtgacctggcggcccgcaacgtcctgctggttaaccggcactacgccaagatcagcgactttggcctctccaaagcactgggtgccgacgacagctactacactgcccgctcagcagggaagtggccgctcaagtggtacgcacccgaatgcatcaacttccgcaagttctccagccgcagcgatgtctggagctatggggtcaccatgtgggaggccttgtcctacggccagaagccctacaagaagatgaaagggccggaggtcatggccttcatcgagcagggcaagcggatggagtgcccaccagagtgtccacccgaactgtacgcactcatgagtgactgctggatctacaagtgggaggatcgccccgacttcctgaccgtggagcagcgcatgcgagcctgttactacagcctg

SEQ ID NO: 7 - protein, 784 aa, Artificial Sequence
  LAT-CAP1
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGPYSDPEELKDKKLFLKSRLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 8 - DNA, 2352 bp, Artificial Sequence
  LAT-CAP1
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctgcggccgccgaggaagctatacttgtgccatgtgtcctgggacttctgctcctccctatacttgcaatgctcatggccctttgcgtgcattgtcacaggcttcccggaagctacgatagtacgtcctccgacagtttgtatccaaggggtattcagttcaagcggccacacacagtagcaccatggccaccagcgtatccccctgtcacaagttacccacctctgtcacagccggacctgctccccatacctagatcaccgcagcctctcggtggtagccaccggaccccatcatctaggagggactccgatggtgcgaattcagtggcgtcctacgaaaatgaggagccggcttgcgaggatgccgacgaagacgaagacgactatcacaacccaggataccttgtcgtactgcctgacagcacaccggctacctctaccgctgcgcccagcgcccccgccttgtccacccctggaatccgcgactcagcgttttcaatggaaagtatagacgattatgtcaacgtccccgaaagtggcgaaagtgctgaagcgagtctcgacgggtctagggaatatgtcaacgtgtctcaggaactgcacccaggagccgcgaagaccgaaccggctgccctctcttcccaggaagcagaagaagtagaggaagagggggcaccagactatgaaaaccttcaggagttgaatggaccttacagcgatccagaggagctgaaggataagaagctgtttctcaagagccgacttattgcagatattgagctcgggtgcggtaatttcggaagtgttcgacagggcgtatacagaatgcggaaaaagcaaattgatgttgccataaaggtactgaaacaagggacagaaaaagctgacaccgaagaaatgatgagggaagcccagatcatgcatcaacttgacaatccatatattgttcgactgataggcgtgtgccaggcggaggccctcatgctggtaatggaaatggcgggtggaggccctctgcataaattcctcgtagggaagcgcgaagaaatacccgtgtctaacgtagctgagcttcttcaccaagtctcaatgggcatgaagtatcttgaagagaaaaatttcgtccaccgagacctggccgctaggaatgtcttgttggtcaatcgacattacgctaaaatatcagactttggactttctaaagccctgggggccgatgacagctactatacagcgcgcagtgcggggaaatggcctttgaaatggtatgctccagagtgcataaattttcggaaattctcatccaggtccgatgtgtggtcctacggggtgaccatgtgggaagccctgtcttacggccagaaaccctacaaaaaaatgaaaggaccagaggtcatggctttcattgaacagggcaaaaggatggaatgtccacctgagtgtcctccggagctctacgcactgatgagcgattgttggatctacaagtgggaggatagacccgacttcttgacggtcgagcaaagaatgagggcatgctattacagcctc

SEQ ID NO: 9 - protein, 867 aa, Artificial Sequence
  LAT-CAP2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGPYSDPEELKDKKLFLKSRPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 10 - DNA, 2601 bp, Artificial Sequence
  LAT-CAP2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctgcggctgcggaagaggcaatactcgtgccctgtgtgctcgggttgttgctcctcccaatccttgcgatgttgatggctttgtgcgtacattgccacagacttcctgggagctacgattctacctcaagtgactcactgtacccaagggggatccagtttaaacgccctcacacagtggcaccctggccaccagcgtatccgccagtcacctcatacccgccgcttagtcagcccgatttgctcccgatacccaggagtcctcagcctcttggggggagccatcggactccttcctctcgaagggattccgacggcgccaattcagtagcatcctatgagaacgaggagccagcttgcgaagacgcagacgaggacgaggatgattaccacaatccggggtacctcgtagttttgcccgactccactcctgcaacgtcaaccgcggcacctagtgcgcccgcactctcaacaccgggtataagagatagcgcattttctatggagagtattgatgactacgtgaatgttcccgaatcaggtgaaagcgctgaggcttcattggatgggtcacgggaatatgtgaacgtttctcaagaactccacccgggtgccgcaaaaacggagcccgcggcattgagcagccaggaagctgaagaggtggaagaggaaggcgcgccggactatgaaaatctgcaggagctcaatggcccttactctgacccggaagaacttaaggataaaaagctgttcctgaaatcaaggccaaatagctcagcctcaaatgcgagcggcgcagctgcaccgactcttccagctcaccaatccaccttgacgcacccccagcgacgaatagatactctcaattcagatggctatacgccggaacccgctcgcatcacgtctccagataagcctcgacccatgcccatggacacctcagtgtacgaaagcccctactccgatccggaggaacttaaggacaaaaaactgtttttgaaaagagataatctgttgattgcggacattgaactcgggtgcggtaatttcggaagcgtcaggcaaggcgtttataggatgaggaaaaagcagatcgatgtcgctattaaggtattgaagcaagggacggagaaggcggacacggaggagatgatgcgagaggctcaaatcatgcatcaactcgacaacccctacattgttagattgattggagtttgtcaagcagaagccttgatgcttgtcatggagatggccgggggcggacccctccacaagtttttggtggggaagcgcgaagaaatcccggtgtctaacgtagcagagcttttgcatcaggttagcatgggtatgaagtatttggaggagaagaacttcgtacatagagatcttgcagctcgcaatgttctcctcgtaaacaggcactacgcaaaaataagtgattttggcctgagtaaggcgctcggcgccgatgattcttactacactgctagaagcgctgggaaatggcccttgaaatggtatgcacctgaatgcattaacttccgaaaatttagttcccggtccgatgtttggtcttacggcgtgactatgtgggaagctctctcctacggccaaaaaccctacaaaaaaatgaagggacctgaggtgatggcgttcattgaacagggtaaacggatggaatgcccacccgaatgcccgcctgaattgtacgcgcttatgtccgactgttggatctacaagtgggaagatcgaccggactttctcaccgtcgaacaacggatgagagcttgttactactctctt

SEQ ID NO: 11 - protein, 21 aa, Artificial Sequence
  CD8 signal sequence
MALPVTALLLPLALLLHAARP

SEQ ID NO: 12 - DNA, 63 bp, Artificial Sequence
  CD8 signal sequence
atggcgctccctgtcaccgcactgcttcttccgctggcactgctgctgcacgctgcacggcct

SEQ ID NO: 13 - DNA, 1602 bp, Artificial Sequence
  SLP-76
atggcactgaggaatgtgccctttcgctcagaggtcctgggctgggaccccgacagccttgctgactatttcaagaagctcaactataaggactgtgagaaggcagtgaagaagtaccacatcgatggggctcgcttcttgaacctgacagaaaatgacatccagaagttccccaagctccgggtgccgattctcagtaagttaagtcaggaaatcaacaagaacgaagagaggaggagcatcttcacacgcaaaccccaagtcccgcggtttcctgaagagacagaaagccacgaagaggacaatgggggctggtcgtcctttgaagaagacgattatgaaagtcccaatgatgaccaggatggggaggatgatggagactatgagtcccccaatgaggaggaagaggcacccgtggaagatgacgcggattatgagccgccaccctccaatgacgaggaagctctgcagaactccatcctgcctgccaagcctttccccaactccaactccatgtacatcgaccggcccccctctgggaaaaccccccagcagcctcctgtgcccccccagagaccgatggccgccctcccgcccccaccagccggccggaatcactcgccactgcccccaccccagaccaaccacgaagaacccagcagaagcagaaaccacaaaacggcaaagctccctgctccttcaatagacagaagcacgaaacctcccctagatcgttcattagctccgtttgatagagaacccttcacactaggaaagaaaccaccattttctgacaagccctcgattccagcgggaaggtcactcggggagcatttacccaagattcaaaagcctcctttaccaccgaccacggaaagacatgaaaggagcagccccctgccagggaagaagccacctgtgccaaagcatggatggggaccagacagaagagagaatgatgaagatgatgtgcatcagagacctttgccccagccagcactacttcctatgagctccaacactttcccttcaagatctactaagccaagtcccatgaaccctctcccatcctctcacatgcctggagcattctcagaaagtaacagcagttttccacagagtgcctccctgccaccatacttctctcaaggccctagcaacagaccacctatcagagccgaaggcagaaacttccccttgccacttccaaacaaacctcggcccccatcccccgcggaggaagagaattcattaaatgaagagtggtacgtttcttatattacccgaccagaggcagaagctgctcttagaaagataaaccaggatggcacatttctggtcagagacagctctaaaaaaacaacaaccaatccatatgtcctcatggtgttgtacaaagataaagtttacaacatccagatccgttatcagaaggaaagtcaagtttacttgttgggaactggactccgagggaaagaggactttctgtctgtgtcagatattattgactacttcaggaaaatgccacttctgctcattgatgggaaaaaccgaggttccagataccagtgcacattaacgcatgctgcagggtacccatag

SEQ ID NO: 14 - protein, 533 aa, Artificial Sequence
  SLP-76
MALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYP

SEQ ID NO: 15 - DNA, 3354 bp, Artificial Sequence
  SLP-76-CAP
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctgcggccgccgaggaagctatacttgtgccatgtgtcctgggacttctgctcctccctatacttgcaatgctcatggccctttgcgtgcattgtcacaggcttcccggaagcgcactgaggaatgtgccctttcgctcagaggtcctgggctgggaccccgacagccttgctgactatttcaagaagctcaactataaggactgtgagaaggcagtgaagaagtaccacatcgatggggctcgcttcttgaacctgacagaaaatgacatccagaagttccccaagctccgggtgccgattctcagtaagttaagtcaggaaatcaacaagaacgaagagaggaggagcatcttcacacgcaaaccccaagtcccgcggtttcctgaagagacagaaagccacgaagaggacaatgggggctggtcgtcctttgaagaagacgattatgaaagtcccaatgatgaccaggatggggaggatgatggagactatgagtcccccaatgaggaggaagaggcacccgtggaagatgacgcggattatgagccgccaccctccaatgacgaggaagctctgcagaactccatcctgcctgccaagcctttccccaactccaactccatgtacatcgaccggcccccctctgggaaaaccccccagcagcctcctgtgcccccccagagaccgatggccgccctcccgcccccaccagccggccggaatcactcgccactgcccccaccccagaccaaccacgaagaacccagcagaagcagaaaccacaaaacggcaaagctccctgctccttcaatagacagaagcacgaaacctcccctagatcgttcattagctccgtttgatagagaacccttcacactaggaaagaaaccaccattttctgacaagccctcgattccagcgggaaggtcactcggggagcatttacccaagattcaaaagcctcctttaccaccgaccacggaaagacatgaaaggagcagccccctgccagggaagaagccacctgtgccaaagcatggatggggaccagacagaagagagaatgatgaagatgatgtgcatcagagacctttgccccagccagcactacttcctatgagctccaacactttcccttcaagatctactaagccaagtcccatgaaccctctcccatcctctcacatgcctggagcattctcagaaagtaacagcagttttccacagagtgcctccctgccaccatacttctctcaaggccctagcaacagaccacctatcagagccgaaggcagaaacttccccttgccacttccaaacaaacctcggcccccatcccccgcggaggaagagaattcattaaatgaagagtggtacgtttcttatattacccgaccagaggcagaagctgctcttagaaagataaaccaggatggcacatttctggtcagagacagctctaaaaaaacaacaaccaatccatatgtcctcatggtgttgtacaaagataaagtttacaacatccagatccgttatcagaaggaaagtcaagtttacttgttgggaactggactccgagggaaagaggactttctgtctgtgtcagatattattgactacttcaggaaaatgccacttctgctcattgatgggaaaaaccgaggttccagataccagtgcacattaacgcatgctgcagggtacccaggaccttacagcgatccagaggagctgaaggataagaagctgtttctcaagagccgacttattgcagatattgagctcgggtgcggtaatttcggaagtgttcgacagggcgtatacagaatgcggaaaaagcaaattgatgttgccataaaggtactgaaacaagggacagaaaaagctgacaccgaagaaatgatgagggaagcccagatcatgcatcaacttgacaatccatatattgttcgactgataggcgtgtgccaggcggaggccctcatgctggtaatggaaatggcgggtggaggccctctgcataaattcctcgtagggaagcgcgaagaaatacccgtgtctaacgtagctgagcttcttcaccaagtctcaatgggcatgaagtatcttgaagagaaaaatttcgtccaccgagacctggccgctaggaatgtcttgttggtcaatcgacattacgctaaaatatcagactttggactttctaaagccctgggggccgatgacagctactatacagcgcgcagtgcggggaaatggcctttgaaatggtatgctccagagtgcataaattttcggaaattctcatccaggtccgatgtgtggtcctacggggtgaccatgtgggaagccctgtcttacggccagaaaccctacaaaaaaatgaaaggaccagaggtcatggctttcattgaacagggcaaaaggatggaatgtccacctgagtgtcctccggagctctacgcactgatgagcgattgttggatctacaagtgggaggatagacccgacttcttgacggtcgagcaaagaatgagggcatgctattacagcctc

SEQ ID NO: 16 - protein, 1118 aa, Artificial Sequence
  SLP-76-CAP
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAEEAILVPCVLGLLLLPILAMLMALCVHCHRLPGSALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYPGPYSDPEELKDKKLFLKSRLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 17 - protein, 810 aa, Artificial Sequence
  28-CAP1
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGSAGSAAGSGEFLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 18 - DNA, 2430 bp, Artificial Sequence
  28-CAP1
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaattgaggtgatgtacccccccccctacctggataatgagaaaagcaacggcaccatcatccacgtgaagggcaagcacctgtgccccagccccttgtttcctggaccttcaaagcccttctgggtgctcgtcgtggtgggcggcgtgctcgcctgctactccctgctggtcaccgtggcttttatcatcttctgggtcggcagctacgactccacctcttctgactccctgtacccccggggcatccagttcaagcgcccccacaccgtggcaccttggcctcctgcctaccctcctgtgaccagctaccctcctctgagccagcctgacctgcttcctatccctcgctctccacaacccctgggcggaagtcaccggacacctagcagcaggcgggactccgatggcgccaacagcgtggccagctacgagaacgaggaacctgcctgtgaagacgccgatgaggacgaggacgactaccacaaccccggttatctggtggtgctgcctgattctacacctgctacaagcaccgccgcccctagcgcccctgctctgagcaccccaggcatcagagattctgccttcagcatggaaagcatcgacgactacgtgaacgtgcctgagagcggcgagagcgccgaggccagcctggacggctctagagaatacgtgaacgtttctcaggagctgcaccccggagctgccaagaccgagcctgccgctctgagcagccaggaggccgaggaagtggaagaggagggcgcccctgattacgagaatctgcaggagctgaacggctctgccggatctgccgccggcagtggcgagttcctgatcgccgacatcgagctgggatgtggcaacttcggcagcgtgcggcagggcgtctacagaatgagaaagaaacagatcgacgtggccatcaaggtgctgaagcagggcacagagaaggccgacaccgaggaaatgatgcgggaagcccagatcatgcaccaactggacaacccttacatcgtgcggctgatcggcgtgtgccaggccgaagctctgatgctggtgatggaaatggccggcggaggccctctgcataagttcctggttggcaaaagagaggaaatccccgtgtccaacgtggccgagctgctgcatcaagtgtccatgggcatgaagtacctggaagagaagaacttcgtgcacagagacctggccgccagaaacgtgctgctggtgaatagacactacgcaaagatcagcgactttggcctgagcaaggccctgggcgctgatgacagctactatacagccagaagcgctggcaagtggcccctgaaatggtacgcccctgagtgcatcaacttcagaaagttcagcagcagatccgacgtttggagctacggcgtgaccatgtgggaagccctgtcttatggccagaagccttataagaagatgaaaggacctgaggtgatggccttcatcgagcagggaaaacggatggaatgccctcctgaatgtcctccagaactgtacgccctgatgagcgactgctggatctacaagtgggaggatagacccgatttcctgaccgtggaacagagaatgcgggcctgctactacagcctc

SEQ ID NO: 19 - protein, 904 aa, Artificial Sequence
  28-CAP2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 20 - DNA, 2712 bp, Artificial Sequence
  28-CAP2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaatagaggtaatgtatcctccaccttatctggataacgaaaagtccaatgggacaatcatccacgtaaaaggaaaacacctctgtccctcaccactcttccccgggcctagcaagcctttctgggttctggtggtcgtcggtggggtgctggcttgctactcactgcttgtgaccgtagcctttatcatcttttgggtgagctatgactctacctcctctgacagcctttatccccggggcatccaatttaagcggccccacaccgtagcaccctggcctccagcgtaccccccagtgaccagctacccaccactttctcaacctgatctgctgcccattccccgctcaccacagcctcttggaggctcccacagaacaccttcatccagacgggattccgacggggcaaactctgtagcatcctatgaaaatgaggagccagcatgcgaggacgcagatgaggacgaggacgattatcataacccgggataccttgtcgtcctgcctgacagtacaccggctacgagcaccgctgctccctccgcccccgcactgagcaccccagggatccgggactccgcatttagcatggagagcattgatgattacgtgaatgtccctgagagcggcgaatccgctgaagctagcttggatggcagcagagagtacgtgaatgtgagtcaggaacttcacccgggggctgccaaaacagagcctgccgcactgtcctcccaagaggcggaggaggttgaggaggagggggcccccgactacgagaacctgcaggaactgaatggcggcgggggctcaccaaattcttcagccagtaatgcctccggggcagctgcaccaaccctcccagctcaccaatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtgtgccaggccgaggctctcatgctggtcatggagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcc

SEQ ID NO: 21 - protein, 1181 aa, Artificial Sequence
  LAT-CAP3
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGEEAILVPCVLGLLLLPILAMLMALCVHCHRLPGSALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYPPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 22 - DNA, 3543 bp, Artificial Sequence
  LAT-CAP3
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggagaagaggctatcctggtgccttgtgtgctggggctgctgctgctgcctatcctggccatgctgatggccctgtgcgtgcactgccacagactgcctggatctgccctgcgcaacgtgcccttccggtcagaggtgctgggctgggaccctgacagcctggccgactacttcaagaagctgaactacaaggattgtgaaaaagccgtgaagaagtaccacatcgacggagccagattcctgaatctgacagagaacgacatccaaaagttccccaagctcagagtgccaatcctgagcaagctgagccaggagatcaacaaaaacgaggaaagacggagcatcttcacccggaagccccaggtgcctagattcccagaagaaaccgagagccacgaggaggacaacggcggatggagcagcttcgaggaagacgattacgagagccctaacgacgaccaggacggagaagatgatggcgactacgagagccctaatgaggaggaggaagccccagttgaggatgacgccgattacgaacccccgccttctaacgatgaggaagccctgcagaacagcatcctgccagctaaaccctttcctaactctaactccatgtacatcgacagacctcccagcggcaaaacacctcagcagcctcctgttcctccacaacgtccaatggctgctctaccgcctccgcctgctggtagaaatcacagccccctgcctccaccacaaaccaaccatgaggagcccagcagaagccggaaccacaagacagccaagctgcctgccccttcaatcgacaggtccaccaagcctccgctggaccggtccctggcccctttcgaccgtgaaccttttaccctggggaaaaaacctcccttcagcgataagcctagcatccccgctggcagatccctgggcgagcacctgcctaagattcagaagccaccactgcctccgaccaccgaaagacacgagagatcttcacctctgcctggcaagaagcctccggtgcccaagcacggctggggccctgacagacgggaaaacgacgaggatgacgtgcatcagcggccactgccccaacctgccctcctgcccatgagctccaataccttccctagccgtagcaccaagccctcccctatgaaccctctgccttcttctcacatgcctggcgccttctctgagagcaacagctcttttcctcaaagcgccagcctccccccttatttcagccaaggccccagcaatagacctcctatcagagccgagggcagaaacttccctctgcctctgcccaacaagccaagacctccgtctcccgctgaagaggagaatagcctgaacgaggaatggtacgtgtcctatatcaccagacctgaagccgaggcagccctgagaaagatcaaccaggatgggaccttcctggtcagagatagctctaagaagaccaccacaaacccctatgtgctgatggtgctgtacaaggacaaagtgtacaacatccagatccggtaccagaaggaaagccaggtgtacctgctgggcaccggcctgagaggcaaagaggacttcctgtctgtgtcagatatcatcgactacttcagaaagatgcctctgctgctgatcgacggcaaaaaccggggcagcagataccagtgcaccctgactcacgccgctgggtaccctcctaattccagcgccagcaacgcctctggcgccgccgctcccaccctgccggctcaccagagcacactgacccaccctcagagacggatcgatacactgaactctgacggctacacccctgagcctgcccggatcacaagcccagataagccaagacccatgcccatggacaccagcgtgtacgagagcccttattccgaccccgaggagctgaaggacaagaagctgttcctgaagagagacaacctgctgatcgccgacatcgagctgggctgcggcaacttcggctctgtgcgccaaggcgtgtaccggatgaggaagaagcagatcgatgtggccatcaaggtgcttaaacagggcacagagaaggccgacaccgaagaaatgatgcgggaggcccagatcatgcaccaactggacaatccttacatcgtgcggctgatcggcgtgtgccaggccgaggccctgatgctggtgatggaaatggccggcggaggccctctgcacaagtttctggtgggcaagcgggaagagatccctgtgtccaacgtggcggaactgctccaccaggtcagcatgggcatgaaatacctggaagagaagaacttcgtgcaccgggacctggctgccagaaatgtgctgcttgtgaacagacactacgccaagatcagcgacttcggcctgtccaaggccctgggcgccgatgatagctactacaccgccagaagcgccggaaaatggcctctgaagtggtatgcccccgagtgcatcaacttcagaaagttcagttcccggtccgacgtgtggtcctatggcgtgaccatgtgggaagccctcagctacggccagaaaccttacaagaaaatgaagggccccgaggtgatggctttcatcgagcagggaaaaagaatggaatgccctccagaatgtcctcctgaactgtacgccctcatgtccgactgctggatctacaagtgggaggatagacctgatttcctgacggtggaacagcggatgagagcctgctactactcactt

SEQ ID NO: 23 - protein, 705 aa, Artificial Sequence
  28-CAP4
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 24 - DNA, 2115 bp, Artificial Sequence
  28-CAP4
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaatagaggtaatgtatcctccaccttatctggataacgaaaagtccaatgggacaatcatccacgtaaaaggaaaacacctctgtccctcaccactcttccccgggcctagcaagcctttctgggttctggtggtcgtcggtggggtgctggcttgctactcactgcttgtgaccgtagcctttatcatcttttgggtgggcggcgggggctcaccaaattcttcagccagtaatgcctccggggcagctgcaccaaccctcccagctcaccaatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtgtgccaggccgaggctctcatgctggtcatggagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcc

SEQ ID NO: 25 - protein, 905 aa, Artificial Sequence
  8-CAP2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 26 - DNA, 2715 bp, Artificial Sequence
  8-CAP2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgttcctatgatagcacatccagcgattccttatacccgagagggatccagtttaagcgacctcacacggttgcaccgtggcccccagcctaccccccagtaacgagttatcctccactgagtcagcctgacctgctccctattccacgatctcctcaacctctgggtggatcccaccgcactccaagctccaggcgcgacagcgacggcgccaactccgtggcttcttatgagaacgaggaacccgcttgtgaggacgctgacgaagacgaggatgactaccataaccccgggtacctggtggtccttcccgactcaacaccagccacgtccactgccgctccttctgcccccgcactctccacccccggcattcgggatagcgcattctctatggagtccatcgacgattatgtcaacgtccccgagtctggcgaaagcgcggaagccagcctagatggcagccgcgaatacgtcaatgtgtcacaggaactgcatcccggcgctgccaaaaccgagccagcggccttaagctcccaggaggccgaagaggtggaagaggaaggtgcaccagactacgagaatttgcaggaactaaatggaggtgggggcagccctaactctagcgcttccaatgcatctggggcagctgcaccgacgttacccgcacaccagagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 27 - protein, 708 aa, Artificial Sequence
  8-CAP4
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 28 - DNA, 2124 bp, Artificial Sequence
  8-CAP4
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaacaactacccccgcccccagaccccctacaccggcaccaactattgcctctcagcctctgtccctgcgcccagaagcttgtcggccagccgccggaggcgcagtgcatacaaggggactggatttcgcttgcgatatctatatctgggctccactggccggcacttgtggggttttgctgctgtccctggtcattacgttgtattgtggcggcgggggctcaccaaattcttcagccagtaatgcctccggggcagctgcaccaaccctcccagctcaccaatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtgtgccaggccgaggctctcatgctggtcatggagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcc

SEQ ID NO: 29 - protein, 905 aa, Artificial Sequence
  8-CAP2 2Ya
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSFDSTSSDSLFPRGIQFKRPHTVAPWPPAFPPVTSFPPLSQPDLLPIPRSPQPLGGSHRTPSSRRDSDGANSVASFENEEPACEDADEDEDDFHNPGYLVVLPDSTPATSTAAPSAPALSTPGIRDSAFSMESIDDYVNVPESGESAEASLDGSREFVNVSQELHPGAAKTEPAALSSQEAEEVEEEGAPDFENLQELNGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 30 - DNA, 2715 bp, Artificial Sequence
  8-CAP2 2Ya
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgttcctttgatagcacatccagcgattccttattcccgagagggatccagtttaagcgacctcacacggttgcaccgtggcccccagccttccccccagtaacgagttttcctccactgagtcagcctgacctgctccctattccacgatctcctcaacctctgggtggatcccaccgcactccaagctccaggcgcgacagcgacggcgccaactccgtggcttcttttgagaacgaggaacccgcttgtgaggacgctgacgaagacgaggatgacttccataaccccgggtacctggtggtccttcccgactcaacaccagccacgtccactgccgctccttctgcccccgcactctccacccccggcattcgggatagcgcattctctatggagtccatcgacgattatgtcaacgtccccgagtctggcgaaagcgcggaagccagcctagatggcagccgcgaattcgtcaatgtgtcacaggaactgcatcccggcgctgccaaaaccgagccagcggccttaagctcccaggaggccgaagaggtggaagaggaaggtgcaccagacttcgagaatttgcaggaactaaatggaggtgggggcagccctaactctagcgcttccaatgcatctggggcagctgcaccgacgttacccgcacaccagagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 31 - protein, 905 aa, Artificial Sequence
  8-CAP 2Yb
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSFDSTSSDSLFPRGIQFKRPHTVAPWPPAFPPVTSFPPLSQPDLLPIPRSPQPLGGSHRTPSSRRDSDGANSVASFENEEPACEDADEDEDDFHNPGYLVVLPDSTPATSTAAPSAPALSTPGIRDSAFSMESIDDFVNVPESGESAEASLDGSREYVNVSQELHPGAAKTEPAALSSQEAEEVEEEGAPDFENLQELNGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 32 - DNA, 2715 bp, Artificial Sequence
  8-CAP2 2Yb
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgttcctttgatagcacatccagcgattccttattcccgagagggatccagtttaagcgacctcacacggttgcaccgtggcccccagccttccccccagtaacgagttttcctccactgagtcagcctgacctgctccctattccacgatctcctcaacctctgggtggatcccaccgcactccaagctccaggcgcgacagcgacggcgccaactccgtggcttcttttgagaacgaggaacccgcttgtgaggacgctgacgaagacgaggatgacttccataaccccgggtacctggtggtccttcccgactcaacaccagccacgtccactgccgctccttctgcccccgcactctccacccccggcattcgggatagcgcattctctatggagtccatcgacgattttgtcaacgtccccgagtctggcgaaagcgcggaagccagcctagatggcagccgcgaatacgtcaatgtgtcacaggaactgcatcccggcgctgccaaaaccgagccagcggccttaagctcccaggaggccgaagaggtggaagaggaaggtgcaccagacttcgagaatttgcaggaactaaatggaggtgggggcagccctaactctagcgcttccaatgcatctggggcagctgcaccgacgttacccgcacaccagagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 33 - protein, 905 aa, Artificial Sequence
  8-CAP2 3Ya
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSFDSTSSDSLFPRGIQFKRPHTVAPWPPAFPPVTSFPPLSQPDLLPIPRSPQPLGGSHRTPSSRRDSDGANSVASFENEEPACEDADEDEDDFHNPGYLVVLPDSTPATSTAAPSAPALSTPGIRDSAFSMESIDDYVNVPESGESAEASLDGSREYVNVSQELHPGAAKTEPAALSSQEAEEVEEEGAPDFENLQELNGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 34 - DNA, 2715 bp, Artificial Sequence
  8-CAP2 3Ya
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgttcctttgatagcacatccagcgattccttattcccgagagggatccagtttaagcgacctcacacggttgcaccgtggcccccagccttccccccagtaacgagttttcctccactgagtcagcctgacctgctccctattccacgatctcctcaacctctgggtggatcccaccgcactccaagctccaggcgcgacagcgacggcgccaactccgtggcttcttttgagaacgaggaacccgcttgtgaggacgctgacgaagacgaggatgacttccataaccccgggtacctggtggtccttcccgactcaacaccagccacgtccactgccgctccttctgcccccgcactctccacccccggcattcgggatagcgcattctctatggagtccatcgacgattatgtcaacgtccccgagtctggcgaaagcgcggaagccagcctagatggcagccgcgaatacgtcaatgtgtcacaggaactgcatcccggcgctgccaaaaccgagccagcggccttaagctcccaggaggccgaagaggtggaagaggaaggtgcaccagacttcgagaatttgcaggaactaaatggaggtgggggcagccctaactctagcgcttccaatgcatctggggcagctgcaccgacgttacccgcacaccagagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 35 - protein, 905 aa, Artificial Sequence
  8-CAP2 3Yb
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSFDSTSSDSLFPRGIQFKRPHTVAPWPPAFPPVTSFPPLSQPDLLPIPRSPQPLGGSHRTPSSRRDSDGANSVASFENEEPACEDADEDEDDFHNPGYLVVLPDSTPATSTAAPSAPALSTPGIRDSAFSMESIDDYVNVPESGESAEASLDGSREFVNVSQELHPGAAKTEPAALSSQEAEEVEEEGAPDYENLQELNGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 36 - DNA, 2715 bp, Artificial Sequence
  8-CAP2 3Yb
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgttcctttgatagcacatccagcgattccttattcccgagagggatccagtttaagcgacctcacacggttgcaccgtggcccccagccttccccccagtaacgagttttcctccactgagtcagcctgacctgctccctattccacgatctcctcaacctctgggtggatcccaccgcactccaagctccaggcgcgacagcgacggcgccaactccgtggcttcttttgagaacgaggaacccgcttgtgaggacgctgacgaagacgaggatgacttccataaccccgggtacctggtggtccttcccgactcaacaccagccacgtccactgccgctccttctgcccccgcactctccacccccggcattcgggatagcgcattctctatggagtccatcgacgattatgtcaacgtccccgagtctggcgaaagcgcggaagccagcctagatggcagccgcgaattcgtcaatgtgtcacaggaactgcatcccggcgctgccaaaaccgagccagcggccttaagctcccaggaggccgaagaggtggaagaggaaggtgcaccagactacgagaatttgcaggaactaaatggaggtgggggcagccctaactctagcgcttccaatgcatctggggcagctgcaccgacgttacccgcacaccagagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 37 - protein, 905 aa, Artificial Sequence
  8-CAP2 3Yc
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSFDSTSSDSLFPRGIQFKRPHTVAPWPPAFPPVTSFPPLSQPDLLPIPRSPQPLGGSHRTPSSRRDSDGANSVASFENEEPACEDADEDEDDFHNPGYLVVLPDSTPATSTAAPSAPALSTPGIRDSAFSMESIDDFVNVPESGESAEASLDGSREYVNVSQELHPGAAKTEPAALSSQEAEEVEEEGAPDYENLQELNGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 38 - DNA, 2715 bp, Artificial Sequence
  8-CAP2 3Yc
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgttcctttgatagcacatccagcgattccttattcccgagagggatccagtttaagcgacctcacacggttgcaccgtggcccccagccttccccccagtaacgagttttcctccactgagtcagcctgacctgctccctattccacgatctcctcaacctctgggtggatcccaccgcactccaagctccaggcgcgacagcgacggcgccaactccgtggcttcttttgagaacgaggaacccgcttgtgaggacgctgacgaagacgaggatgacttccataaccccgggtacctggtggtccttcccgactcaacaccagccacgtccactgccgctccttctgcccccgcactctccacccccggcattcgggatagcgcattctctatggagtccatcgacgattttgtcaacgtccccgagtctggcgaaagcgcggaagccagcctagatggcagccgcgaatacgtcaatgtgtcacaggaactgcatcccggcgctgccaaaaccgagccagcggccttaagctcccaggaggccgaagaggtggaagaggaaggtgcaccagactacgagaatttgcaggaactaaatggaggtgggggcagccctaactctagcgcttccaatgcatctggggcagctgcaccgacgttacccgcacaccagagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 39 - protein, 708 aa, Artificial Sequence
  8-CAP4 ZAPAS1
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVAEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 40 - DNA, 2124 bp, Artificial Sequence
  8-CAP4 ZAPAS1
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaacaactacccccgcccccagaccccctacaccggcaccaactattgcctctcagcctctgtccctgcgcccagaagcttgtcggccagccgccggaggcgcagtgcatacaaggggactggatttcgcttgcgatatctatatctgggctccactggccggcacttgtggggttttgctgctgtccctggtcattacgttgtattgtggcggcgggggctcaccaaattcttcagccagtaatgcctccggggcagctgcaccaaccctcccagctcaccaatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtgtgccaggccgaggctctcatgctggtcgccgagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcc

SEQ ID NO: 41 - protein, 708 aa, Artificial Sequence
  8-CAP4 ZAPAS2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVVQAEALMLVAEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 42 - DNA, 2124 bp, Artificial Sequence
  8-CAP4 ZAPAS2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaacaactacccccgcccccagaccccctacaccggcaccaactattgcctctcagcctctgtccctgcgcccagaagcttgtcggccagccgccggaggcgcagtgcatacaaggggactggatttcgcttgcgatatctatatctgggctccactggccggcacttgtggggttttgctgctgtccctggtcattacgttgtattgtggcggcgggggctcaccaaattcttcagccagtaatgcctccggggcagctgcaccaaccctcccagctcaccaatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtggtgcaggccgaggctctcatgctggtcgccgagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcc

SEQ ID NO: 43 - protein, 947 aa, Artificial Sequence
  8-41BB CAP2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 44 - DNA, 2841 bp, Artificial Sequence
  8-41BB CAP2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgtaaacggggcagaaagaaactcctgtatatattcaaacaaccatttatgagaccagtacaaactactcaagaggaagatggctgtagctgccgatttccagaagaagaagaaggaggatgtgaactgtcctatgatagcacatccagcgattccttatacccgagagggatccagtttaagcgacctcacacggttgcaccgtggcccccagcctaccccccagtaacgagttatcctccactgagtcagcctgacctgctccctattccacgatctcctcaacctctgggtggatcccaccgcactccaagctccaggcgcgacagcgacggcgccaactccgtggcttcttatgagaacgaggaacccgcttgtgaggacgctgacgaagacgaggatgactaccataaccccgggtacctggtggtccttcccgactcaacaccagccacgtccactgccgctccttctgcccccgcactctccacccccggcattcgggatagcgcattctctatggagtccatcgacgattatgtcaacgtccccgagtctggcgaaagcgcggaagccagcctagatggcagccgcgaatacgtcaatgtgtcacaggaactgcatcccggcgctgccaaaaccgagccagcggccttaagctcccaggaggccgaagaggtggaagaggaaggtgcaccagactacgagaatttgcaggaactaaatggaggtgggggcagccctaactctagcgcttccaatgcatctggggcagctgcaccgacgttacccgcacaccagagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 45 - protein, 946 aa, Artificial Sequence
  8-28 CAP2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 46 - DNA, 2838 bp, Artificial Sequence
  8-28 CAP2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgtaggagcaagaggtccaggctgctgcacagcgactacatgaatatgacccccagaaggcccggccccaccagaaagcactatcagccctacgccccccccagggactttgccgcctacaggagctcctatgatagcacatccagcgattccttatacccgagagggatccagtttaagcgacctcacacggttgcaccgtggcccccagcctaccccccagtaacgagttatcctccactgagtcagcctgacctgctccctattccacgatctcctcaacctctgggtggatcccaccgcactccaagctccaggcgcgacagcgacggcgccaactccgtggcttcttatgagaacgaggaacccgcttgtgaggacgctgacgaagacgaggatgactaccataaccccgggtacctggtggtccttcccgactcaacaccagccacgtccactgccgctccttctgcccccgcactctccacccccggcattcgggatagcgcattctctatggagtccatcgacgattatgtcaacgtccccgagtctggcgaaagcgcggaagccagcctagatggcagccgcgaatacgtcaatgtgtcacaggaactgcatcccggcgctgccaaaaccgagccagcggccttaagctcccaggaggccgaagaggtggaagaggaaggtgcaccagactacgagaatttgcaggaactaaatggaggtgggggcagccctaactctagcgcttccaatgcatctggggcagctgcaccgacgttacccgcacaccagagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 47 - protein, 750 aa, Artificial Sequence
  8-41BB CAP4
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 48 - DNA, 2250 bp, Artificial Sequence
  8-41BB CAP4
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaacaactacccccgcccccagaccccctacaccggcaccaactattgcctctcagcctctgtccctgcgcccagaagcttgtcggccagccgccggaggcgcagtgcatacaaggggactggatttcgcttgcgatatctatatctgggctccactggccggcacttgtggggttttgctgctgtccctggtcattacgttgtattgtaaacggggcagaaagaaactcctgtatatattcaaacaaccatttatgagaccagtacaaactactcaagaggaagatggctgtagctgccgatttccagaagaagaagaaggaggatgtgaactgggcggcgggggctcaccaaattcttcagccagtaatgcctccggggcagctgcaccaaccctcccagctcaccaatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtgtgccaggccgaggctctcatgctggtcatggagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcc

SEQ ID NO: 49 - protein, 749 aa, Artificial Sequence
  8-28 CAP4
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSGGGGSPNSSASNASGAAAPTLPAHQSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 50 - DNA, 2256 bp, Artificial Sequence
  8-28 CAP4
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaacaactacccccgcccccagaccccctacaccggcaccaactattgcctctcagcctctgtccctgcgcccagaagcttgtcggccagccgccggaggcgcagtgcatacaaggggactggatttcgcttgcgatatctatatctgggctccactggccggcacttgtggggttttgctgctgtccctggtcattacgttgtattgtaggagcaagaggtccaggctgctgcacagcgactacatgaatatgacccccagaaggcccggccccaccagaaagcactatcagccctacgccccccccagggactttgccgcctacaggagcggcggcgggggctcaccaaattcttcagccagtaatgcctccggggcagctgcaccaaccctcccagctcaccaatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtgtgccaggccgaggctctcatgctggtcatggagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcctaagtcgac

SEQ ID NO: 51 - protein, 705 aa, Artificial Sequence
  CAP4.2 construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 52 - DNA, 2118 bp, Artificial Sequence
  CAP4.2 construct
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaatagaggtaatgtatcctccaccttatctggataacgaaaagtccaatgggacaatcatccacgtaaaaggaaaacacctctgtccctcaccactcttccccgggcctagcaagcctttctgggttctggtggtcgtcggtggggtgctggcttgctactcactgcttgtgaccgtagcctttatcatcttttgggtgggcggcgggggctcaccaaattcttcagccagtaatgcctccggggcagctgcaccaaccctcccagctcacccatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtgtgccaggccgaggctctcatgctggtcatggagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcctga

SEQ ID NO: 53 - protein, 741 aa, Artificial Sequence
  CAP4.6 construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 54 - DNA, 2226 bp, Artificial Sequence
  CAP4.6 construct
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaattgaagtgatgtacccccctccatacctggacaacgagaaatccaatggtaccatcatccacgttaagggcaagcacctctgcccctcacctctgttccctgggccttctaaacctttttgggtactggtcgtcgtgggcggagtgttggcatgttattcactgctcgtgaccgtcgcctttatcatcttttgggtgcgctcaaaacgatctcggctcttgcattctgactatatgaatatgactcccaggcgccctggaccaactcgcaagcactatcagccctatgcgcccccgcgggactttgccgcctaccgctctcctaacagctccgcgtcaaatgcttccggcgccgcggcccctactctccccgcacatccctctaccctcacgcacccgcagagacgcatcgacactctgaacagtgatggctacacccctgagcccgcaaggattacaagcccagataaacccagaccgatgcccatggatacctccgtatacgaatccccctattcagaccccgaggagctcaaggacaaaaagctctttcttaagcgagataacctgctgattgctgatatcgagctgggatgtggtaacttcgggagcgtacgccagggcgtgtacagaatgcggaagaagcaaatagatgtggctataaaagtgctgaagcagggcactgaaaaagccgacaccgaagaaatgatgcgagaggctcaaatcatgcatcagcttgacaacccatacattgttagattgatcggtgtgtgtcaagctgaagcactcatgctggtcatggagatggccggaggtggccctcttcacaagtttctcgtcggcaaaagggaggagatccccgtgagtaatgtggcagagctgctccaccaggtgtctatgggaatgaagtatctggaagaaaaaaatttcgtgcacagagacctggccgcacgcaacgtactcctcgttaatagacactacgccaagatatcagattttgggctcagcaaggctttgggggcggacgactcttactacacagctcgctccgccggcaaatggcctctgaagtggtacgctcccgagtgcatcaattttaggaaattctcatcaagaagcgacgtctggtcatatggggttacgatgtgggaggcactctcctacggccagaagccctacaaaaagatgaaaggtccagaggtgatggctttcattgagcaggggaagcgcatggagtgtcctccagaatgccctccagaactgtatgctctcatgtccgactgctggatttataaatgggaggacaggcccgacttcctcaccgtcgagcaacgaatgagagcgtgctattatagccttgcctcaaaagttgagggtcctcctgggtccactcagaaggcagaggccgcatgtgcgtga

SEQ ID NO: 55 - protein, 995 aa, Artificial Sequence
  CAP4.7 construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSMPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 56 - DNA, 2988 bp, Artificial Sequence
  CAP4.,7 construct
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaatcgaagtgatgtatcccccaccttacctcgacaacgaaaagtccaatggcacaataattcacgtcaaaggcaagcatctgtgtccgtcccctctgtttcccggacctagtaagccattctgggtgttggtcgtggtggggggcgtgctcgcgtgttattccctgctggtcactgtggcatttattatattctgggttagatcaaagcgctctcgcctcctccacagtgactacatgaacatgacgccccggcgcccgggccctactagaaaacactatcagccctatgcaccacccagggatttcgccgcttacaggagtatgcccgacccggcagctcatctgccattcttctacgggagtatctccagggccgaagcagaggagcacctcaaactcgccggtatggctgacggactgttcctcctcagacagtgcttgagaagtctcggcggttatgtgctgagcctcgtgcacgacgtgcggttccaccacttccccatagaacggcaactgaatgggacatacgccatcgccggcggcaaggctcattgcggacctgccgagctgtgcgaattctacagccgggaccccgacggcctgccatgtaatttgcggaagccttgcaaccgcccaagcgggctggagcctcagcctggcgtcttcgattgtcttcgggatgccatggttagggattatgtccggcagacatggaaactggagggtgaagcactggagcaggctattattagccaagcaccccaggttgagaagctgattgctactaccgcccatgaaaggatgccatggtaccactcaagcctgactcgcgaggaagccgaaagaaagttgtactccggggcacagaccgacggcaagttcctgcttcggccccgcaaagagcagggcacatacgcactctctctgatctatggaaagacagtttaccactacctcatctcccaggacaaggcaggcaagtactgcatccccgagggtaccaagttcgacacgctgtggcagctggttgaataccttaagctcaaggctgacggtctgatctactgcctgaaggaggcatgcccaaatagctcagctagcaatgcttccggtgccgccgcaccaacccttcctgcccacccctctaccctcacacatccacagaggcgcatcgacaccctgaacagcgatgggtataccccagaacctgctcggatcacatcaccagataaaccgaggcctatgcctatggatacaagcgtgtacgagagcccttacagtgacccagaggagctgaaggacaagaagctcttcctgaaacgagacaacctgttgatagccgacattgagcttggctgtggcaatttcggaagcgtccggcagggggtctataggatgagaaagaaacaaattgacgttgccatcaaggtcctcaagcaaggtacggagaaggccgacaccgaagaaatgatgcgggaggcccagataatgcaccagttggataatccttacatcgtgagattgatcggtgtctgtcaggctgaagcactgatgctggtgatggagatggcaggtggagggcccctgcataagttcctcgtgggaaagagggaggagatacctgtctccaatgtagccgaactcctgcatcaagtgtcaatgggcatgaagtacctcgaggagaaaaactttgtccaccgcgacttggcggctcgaaatgtgttgctggtgaaccggcactacgccaaaatcagtgactttggactgtccaaggccctcggtgctgacgacagttattacacagcaagatctgctgggaagtggcctcttaaatggtatgctcctgagtgcataaattttaggaaattctctagtcggtctgacgtttggtcctatggggtcacaatgtgggaggcgctgtcttatggccagaaaccctataagaaaatgaaagggccagaggtaatggcttttatcgaacaggggaaacgaatggagtgtccccccgagtgtccgcccgaactctacgctctgatgagcgattgctggatttacaagtgggaggaccggcccgatttcctcactgttgagcaacgaatgagagcctgctactactccttggctagtaaggttgaggggccgcctgggtcaacccaaaaggccgaggccgcttgtgcttga

SEQ ID NO: 57 - protein, 741 aa, Artificial Sequence
  CAP4.8 construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVRSKRSRLLHSDFMNMTARRAGPTRKHYQAYAAPRDFAAYRSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 58 - DNA, 2226 bp, Artificial Sequence
  CAP4.8 construct
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaatcgaagtcatgtaccctcccccttatcttgacaatgagaaaagcaacggcacgattattcacgtgaaagggaagcatctctgcccctctccgctgttcccaggtcccagtaagccattctgggtgcttgtggttgtggggggcgtcctggcttgctactctcttctggtcacagtcgcttttattatattttgggtaagatccaagcgaagccgcctgctgcattccgactttatgaacatgaccgcaagacgggccggtcctaccagaaaacactatcaggcctatgcagctccccgggattttgccgcctaccgatctccgaatagtagcgcgagtaacgcctctggagcagccgcacctacgctccctgctcaccccagtacgctcacccacccccagagaaggatcgacacactcaactccgatgggtacacaccagaaccagcgaggattacaagtcctgataaacctcgcccgatgcccatggatacatctgtgtatgagtccccctactctgaccccgaggaacttaaggacaagaagctcttcctgaagagagataacttgctcatcgcggatatcgagcttggatgcggcaactttggcagtgtgcgacagggtgtctatagaatgcgcaaaaaacagatagatgtcgctattaaagttctgaagcagggcacagaaaaggcagacactgaggagatgatgagggaggcacaaatcatgcaccaactcgataatccctacattgtccgccttattggagtctgtcaggcggaagccctcatgctggtaatggagatggccggaggcggtccgctgcacaaatttctcgtcgggaagagggaggaaatcccggtgtccaatgtggcagagctccttcatcaggtgtctatgggcatgaagtatctcgaggagaagaattttgtgcacagagacttggctgcccgaaacgtgctgctggtcaaccggcattatgcgaagatctccgatttcggattgtctaaagctctgggagctgacgattcatactatacagccaggagcgcgggaaagtggcctctcaaatggtatgcccctgaatgtatcaatttcaggaaatttagtagtcgatccgacgtctggagctatggagtgacaatgtgggaggccctgtcttacggtcaaaagccatacaagaagatgaaaggtcccgaagtcatggcattcatcgagcaggggaagaggatggagtgtccccctgaatgtccacccgaactgtatgcgctgatgtcagactgttggatctacaaatgggaggaccgcccggactttctcacggtggagcagcgcatgagagcgtgctactatagcttggcttccaaagtggagggccccccgggctcaacccaaaaggctgaggctgcgtgtgcttga

SEQ ID NO: 59 - protein, 995 aa, Artificial Sequence
  CAP4.9 construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVRSKRSRLLHSDFMNMTARRAGPTRKHYQAYAAPRDFAAYRSMPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 60 - DNA, 2988 bp, Artificial Sequence
  CAP4.9 construct
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaatcgaggtcatgtatccccctccatacctggataatgagaagtccaatggcaccataattcacgtcaagggcaaacacttgtgccccagtcctcttttccccggcccttccaaaccgttctgggtgcttgtggtggtcggaggcgtgcttgcctgctactctctcctcgttaccgtagcttttatcatcttctgggtgagatccaagaggtcacgcctgctgcacagcgatttcatgaacatgaccgcccggagagcaggtccaaccaggaagcactaccaggcctacgccgccccccgggactttgctgcataccgctccatgcccgacccagccgcgcacctgcccttcttttatgggtcaatatccagagctgaagcggaagagcatcttaaacttgccggcatggccgacggactgttcctgcttcggcagtgcctccgatctcttgggggctacgtgctctccctggtgcacgacgttcggtttcaccacttccctatagagaggcagctcaacgggacctatgctatagcagggggtaaggctcattgcggacccgccgagctttgtgaattttatagtagagaccccgatggacttccctgtaatcttcgaaaaccatgtaatcgccctagtggcctggagccccagccaggcgtattcgattgcctcagggacgccatggtcagagactacgtcagacaaacatggaagctcgaaggggaggccttggaacaagctatcatcagccaggccccccaggtggagaagctgattgctaccacggcccacgaacggatgccctggtatcatagctccctgacaagggaggaagctgagcgaaaactttacagtggagctcagacggatggcaaattccttcttcgcccgcggaaggaacagggtacctacgctttgtcattgatttatggaaagactgtttaccactacttgatttctcaagacaaggccggtaaatactgcatacctgagggcaccaaatttgatacattgtggcaacttgtggaatatcttaaactgaaagctgatggtctcatatattgtcttaaagaagcctgtcctaattcctctgccagtaacgcctctggtgcggctgcgccaaccttgcctgcccacccaagcaccttgacccatccccagagacgcatcgataccctgaactcagacggatacacaccggagccagcaaggattacttcccccgacaagccccgccccatgcccatggatactagtgtgtacgagtccccatattccgacccagaggagcttaaagataaaaagctgttcctgaagcgggataacctgctgatagcggatatagaactggggtgcggcaattttggatcagtgagacagggggtgtatcggatgcgcaagaagcagatcgacgtggccattaaagtgctcaaacaaggaaccgagaaagcagatacagaagagatgatgcgcgaagcccaaataatgcaccagctggacaatccttatatcgtgaggttgattggcgtgtgtcaggcagaggccttgatgctcgtgatggaaatggcgggagggggcccgttgcacaaattcctggtcgggaagcgcgaagaaatacccgtgagtaacgtagccgagttgctgcaccaggtgtctatgggaatgaaatatctggaggagaagaactttgttcatcgggacctcgccgctcgcaacgtacttctcgtgaatcggcactatgcaaaaatttctgactttgggctttctaaggcgctcggagcggatgactcatattacactgccagatcagccggcaagtggcccctcaagtggtacgcaccagaatgcatcaatttccgcaaattctcatccagatcagacgtgtggtcctacggtgtcaccatgtgggaagcactgagctacgggcagaagccatacaaaaagatgaaaggacctgaggtcatggcctttatcgagcaaggaaagagaatggaatgccctccagaatgtccccccgagctgtacgccctgatgtccgattgttggatatataaatgggaagacaggcccgactttcttacagttgaacaaaggatgcgggcctgctactattcacttgccagcaaggtcgaaggccctcctggatcaactcagaaggccgaggcagcttgtgcttaa

SEQ ID NO: 61 - protein, 959 aa, Artificial Sequence
  CAP4.10 construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVGGGGSMPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 62 - DNA, 2880 bp, Artificial Sequence
  CAP4.10 construct
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaatcgaagtcatgtatcctcccccttacctggataacgagaagtccaacggcaccattatccacgtgaaagggaaacatttgtgtccctcccctcttttccccggcccttccaagcccttttgggtcctcgtggtggtgggaggagtgcttgcatgttacagcctcctcgttaccgtggcatttataattttttgggtcggcggggggggatccatgcccgatcctgccgcccacctgcctttcttttatgggtctatttccagggccgaggccgaggagcatttgaagctcgcaggtatggccgacggcttgttcctcctgcggcaatgtttgaggagtcttgggggctatgtcctgagtctggtccacgatgtgcgcttccaccacttcccgattgaacgccagctgaacggcacctatgccatcgcaggcgggaaagcccactgcggccccgccgaactttgtgagttttatagcagggaccctgacggattgccatgtaatctgcggaagccttgcaaccggccctctggtctggagccccagcccggggtattcgactgtctgcgcgacgccatggtgcgagattacgttcgacagacttggaagctggagggagaggctctggagcaggcaatcatcagtcaggcgccacaggtggaaaagcttattgctactaccgcccatgagaggatgccatggtatcacagttccctcaccagagaggaggccgagcgcaaactttattccggagctcagacagacgggaaattcctgctgcggccgcgcaaggaacaaggaacgtacgcccttagcctcatttatggaaaaaccgtctatcactatcttatcagccaggacaaggcggggaaatattgtattcccgaagggaccaaattcgacaccttgtggcagctggtggaatatctcaagcttaaagcagacggactgatctattgtctcaaggaagcctgccccaactcttccgcatccaacgcctctggcgcagccgcaccgacactccctgcgcaccccagcaccttgacccatccacagcgccggatagatacactcaattcagatggctatactcctgagcctgcgcgcattacctcacctgataaacctagaccgatgcctatggacaccagcgtatacgagagcccttattctgacccagaagagctgaaggacaagaaactgttcctcaaacgagataacctgcttattgctgacatcgagcttggatgtggaaactttggaagcgtcaggcagggggtatatagaatgcgaaagaaacaaattgacgtggcaattaaggtcctgaaacagggaactgagaaggccgacactgaagaaatgatgcgggaagcgcagataatgcatcagttggacaatccatacatagtgaggctcattggggtttgccaagccgaagcactgatgctcgtcatggagatggccggtggtggacctcttcataagttcctggtgggcaagcgcgaagaaattcccgtgagcaacgtggcggaactcctccatcaagtcagtatggggatgaagtatctggaggaaaaaaacttcgtacaccgagacctcgccgctcgcaacgtgctgctggtgaataggcactacgcaaagatcagcgacttcgggctgagtaaggcactgggcgccgatgatagctactacaccgcgcgcagtgccgggaagtggcctttgaaatggtacgctcctgagtgcattaattttagaaagtttagctcaagatctgatgtgtggagttacggagtaacaatgtgggaggccctgtcttacgggcagaagccctacaaaaagatgaaaggccctgaggtgatggccttcattgagcagggcaagaggatggagtgtcctccggaatgcccaccagagctgtacgctttgatgagcgattgttggatttataaatgggaagaccgccccgacttcctgaccgtggagcagcgcatgagggcgtgctactatagtctggcttccaaagtggaggggccccctggatcaacccagaaggctgaggccgcgtgtgcctaa

SEQ ID NO: 63 - protein, 957 aa, Artificial Sequence
  CAP4.11 construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCMPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 64 - DNA, 2874 bp, Artificial Sequence
  CAP4.11 construct
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaactacaactcccgctccacggcctccaacccctgcgcccacgatcgcttcccagcctctgagcttgagacctgaggcgtgccgacccgctgcagggggtgccgtacacacgaggggtctcgacttcgcttgtgacatctacatttgggccccactcgccgggacatgcggagtactgctcctgtcactggtcatcaccctgtactgcatgcccgatccggcagcacacctcccttttttttatggcagcatcagcagagctgaagccgaagaacatcttaaattggccggcatggctgatggactgttcctgctgcggcagtgtctgaggtcactgggcggttacgtactcagtctcgtgcacgatgttaggttccaccatttcccaattgaaagacaattgaatggaacatatgctattgcaggaggtaaagcacactgtggacccgccgagctctgtgagttttacagccgcgacccagacggcctgccctgtaatctgcggaagccctgtaatcgccccagcgggcttgaaccacagccaggtgtgtttgactgcctgagagacgcaatggtaagagactacgtccggcagacctggaaactcgagggcgaggcgttggagcaagcaatcatatctcaagcgccccaggtcgaaaaattgattgccacgaccgcccacgagcgaatgccctggtaccacagttcactcaccagagaggaagctgaacgaaaactgtatagcggagcccagacagacgggaagttcctccttcggccacggaaagagcaggggacttacgcactgtcattgatttacggcaagacagtgtaccactacctgattagtcaggacaaggccggcaagtattgtattccagaaggtacaaagtttgacactctgtggcagctggtggaataccttaagcttaaggccgacggcctcatctattgtctgaaagaagcatgccctaacagtagtgctagcaacgcgagcggggccgctgcacccacccttcctgcccacccatccacgcttacccatccccagaggaggattgataccctcaattctgatggctatactccagagcccgccagaattacctctccagacaaaccacgacctatgcccatggacactagcgtctacgagagcccctatagcgatcccgaggagctgaaagataagaaattgtttctgaagagagacaacctgcttatcgccgacatcgagctgggatgcggaaacttcggctccgtcagacaaggcgtgtaccgaatgcgaaagaagcagatcgacgtggctatcaaagtactgaaacagggcacagagaaagctgatactgaagagatgatgcgcgaagcacaaataatgcatcagctcgacaacccgtacatagtgcggctcattggtgtttgtcaggccgaggcactgatgttggtgatggagatggctggaggggggcccctccacaaattcctggtcggcaagcgagaggaaatccctgttagtaacgtggcggagctcctgcaccaggtcagcatgggaatgaagtacctcgaggagaaaaactttgttcaccgagacttggcagcacgcaacgtgctgttggttaacagacattacgcaaaaatcagcgatttcggactgtccaaggccctcggcgctgacgattcatattatacagcccgctccgctgggaagtggccgctcaagtggtatgctcctgaatgtataaactttcgcaagttcagctctagatctgacgtttggagttacggcgtgactatgtgggaggctctctcctacggacagaagccttacaaaaagatgaagggccccgaagtgatggcctttatcgagcagggaaagagaatggaatgcccccccgaatgccccccggaactctacgccctgatgtccgactgctggatctacaagtgggaagatcggcccgacttccttacggtagagcagcgcatgcgggcttgttactactccctggcctctaaagttgaaggtccgcccggcagtacccaaaaagctgaagccgcatgcgcgtag

SEQ ID NO: 65 - protein, 999 aa, Artificial Sequence
  CAP4.12 construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELMPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 66 - DNA, 3000 bp, Artificial Sequence
  CAP4.12 construct
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaaccaccactcccgcgccgcgaccccccaccccagcccccaccatcgcaagccagcctttgagtcttcgccctgaggcatgccgaccagctgcgggaggcgcagtccatactcgcggtcttgattttgcctgtgatatctatatttgggcaccccttgccggcacttgcggagtgttgctcttgtccctggtaatcactctgtactgcaaaaggggacgaaagaaattgctctatattttcaagcagccgtttatgagacctgtccaaaccacccaggaagaggatggctgttcctgcagattccccgaggaggaggagggaggctgcgaattgatgccagacccagccgcgcacctgccctttttttacggtagcatttcacgggcagaagctgaggagcatcttaagctggccggcatggctgacggtctctttctgctccggcagtgcctgagatctctgggcggatatgtcctgtcactcgtgcacgacgttagatttcatcatttccctatagaacggcaattgaacggaacttacgcgatcgccgggggtaaggcacactgtggacctgccgaactgtgtgagttttattcaagggacccggatggcctgccctgcaacttgagaaagccgtgcaaccggccaagtgggctcgagccacagcctggtgtcttcgactgtctcagagatgctatggtacgagactacgtgcgccagacgtggaaactcgagggtgaggccctggagcaggcaatcatttcccaggcccctcaagttgagaagttgatagctactaccgcacacgagagaatgccttggtaccacagctctctgactcgggaggaggcagaacgaaaattgtatagcggggcccagactgatggcaagttccttctgcgcccgagaaaggaacagggcacttacgctctcagcctgatctacggaaaaacagtataccactatctcattagtcaagacaaggccggaaaatactgcatccctgagggaacaaagtttgatacactgtggcagcttgtggagtacctgaagctgaaggccgatggccttatttactgtttgaaggaggcatgccctaacagcagcgcctcaaacgcaagcggagcagccgctcccacactgcccgcccaccctagtacactgactcatccacaacgacggatcgatacactgaatagcgacggatacaccccagaacctgctaggataacatcccccgacaaaccccgccctatgcctatggatacctccgtctatgagtccccatatagcgacccggaagagctcaaggataagaagctgttcttgaaacgggacaaccttttgattgccgacatcgagctcggctgcgggaactttggatcagttagacagggcgtataccgcatgcggaagaaacaaatcgatgtcgccattaaagtgctgaagcaaggtactgagaaagccgataccgaggaaatgatgagagaggctcagatcatgcatcaactcgacaacccctatatcgtgcggcttatcggcgtctgtcaggcggaggcgctgatgctggtgatggaaatggcgggaggcggacccctccataaattcctggttggcaagcgagaagaaatccctgtcagtaatgtcgcagagctcctgcaccaggtatcaatgggcatgaaatacctggaagagaagaattttgtgcacagggacctggccgctcgcaatgtgctccttgttaaccggcattacgcaaaaatcagcgacttcggactcagcaaggccctcggcgcggatgactcttactacacagctcggtccgctggtaaatggccactgaagtggtacgctcctgagtgcatcaacttccggaaatttagcagtaggtcagatgtctggagttacggcgtgactatgtgggaggctctcagttatggacaaaaaccttataaaaaaatgaagggacccgaagttatggcgttcatagaacagggtaaacgaatggagtgcccacctgagtgtcctcccgagctgtatgccctgatgtctgattgctggatctacaaatgggaagaccggcctgactttctgacagtcgagcagcggatgagagcatgctactatagcctggcctcaaaggtcgaggggcctcccggcagcacccagaaagccgaagcggcctgcgcataa

SEQ ID NO: 67 - protein, 4 aa, Artificial Sequence
  CD28 regulatory sequence
YMNM

SEQ ID NO: 68 - protein, 4 aa, Artificial Sequence
  CD28 regulatory sequence
PRRP

SEQ ID NO: 69 - protein, 4 aa, Artificial Sequence
  CD28 regulatory sequence
PYAP

SEQ ID NO: 70 - protein, 904 aa, Artificial Sequence
  28-CAP2-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 71 - DNA, 2712 bp, Artificial Sequence
  28-CAP2-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaatagaggtaatgtatcctccaccttatctggataacgaaaagtccaatgggacaatcatccacgtaaaaggaaaacacctctgtccctcaccactcttccccgggcctagcaagcctttctgggttctggtggtcgtcggtggggtgctggcttgctactcactgcttgtgaccgtagcctttatcatcttttgggtgagctatgactctacctcctctgacagcctttatccccggggcatccaatttaagcggccccacaccgtagcaccctggcctccagcgtaccccccagtgaccagctacccaccactttctcaacctgatctgctgcccattccccgctcaccacagcctcttggaggctcccacagaacaccttcatccagacgggattccgacggggcaaactctgtagcatcctatgaaaatgaggagccagcatgcgaggacgcagatgaggacgaggacgattatcataacccgggataccttgtcgtcctgcctgacagtacaccggctacgagcaccgctgctccctccgcccccgcactgagcaccccagggatccgggactccgcatttagcatggagagcattgatgattacgtgaatgtccctgagagcggcgaatccgctgaagctagcttggatggcagcagagagtacgtgaatgtgagtcaggaacttcacccgggggctgccaaaacagagcctgccgcactgtcctcccaagaggcggaggaggttgaggaggagggggcccccgactacgagaacctgcaggaactgaatggcggcgggggctcaccaaattcttcagccagtaatgcctccggggcagctgcaccaaccctcccagctcacccatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtgtgccaggccgaggctctcatgctggtcatggagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcc

SEQ ID NO: 72 - protein, 1181 aa, Artificial Sequence
  LAT-CAP3-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGEEAILVPCVLGLLLLPILAMLMALCVHCHRLPGSALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYPPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 73 - DNA, 3543 bp, Artificial Sequence
  LAT-CAP3-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggagaagaggctatcctggtgccttgtgtgctggggctgctgctgctgcctatcctggccatgctgatggccctgtgcgtgcactgccacagactgcctggatctgccctgcgcaacgtgcccttccggtcagaggtgctgggctgggaccctgacagcctggccgactacttcaagaagctgaactacaaggattgtgaaaaagccgtgaagaagtaccacatcgacggagccagattcctgaatctgacagagaacgacatccaaaagttccccaagctcagagtgccaatcctgagcaagctgagccaggagatcaacaaaaacgaggaaagacggagcatcttcacccggaagccccaggtgcctagattcccagaagaaaccgagagccacgaggaggacaacggcggatggagcagcttcgaggaagacgattacgagagccctaacgacgaccaggacggagaagatgatggcgactacgagagccctaatgaggaggaggaagccccagttgaggatgacgccgattacgaacccccgccttctaacgatgaggaagccctgcagaacagcatcctgccagctaaaccctttcctaactctaactccatgtacatcgacagacctcccagcggcaaaacacctcagcagcctcctgttcctccacaacgtccaatggctgctctaccgcctccgcctgctggtagaaatcacagccccctgcctccaccacaaaccaaccatgaggagcccagcagaagccggaaccacaagacagccaagctgcctgccccttcaatcgacaggtccaccaagcctccgctggaccggtccctggcccctttcgaccgtgaaccttttaccctggggaaaaaacctcccttcagcgataagcctagcatccccgctggcagatccctgggcgagcacctgcctaagattcagaagccaccactgcctccgaccaccgaaagacacgagagatcttcacctctgcctggcaagaagcctccggtgcccaagcacggctggggccctgacagacgggaaaacgacgaggatgacgtgcatcagcggccactgccccaacctgccctcctgcccatgagctccaataccttccctagccgtagcaccaagccctcccctatgaaccctctgccttcttctcacatgcctggcgccttctctgagagcaacagctcttttcctcaaagcgccagcctccccccttatttcagccaaggccccagcaatagacctcctatcagagccgagggcagaaacttccctctgcctctgcccaacaagccaagacctccgtctcccgctgaagaggagaatagcctgaacgaggaatggtacgtgtcctatatcaccagacctgaagccgaggcagccctgagaaagatcaaccaggatgggaccttcctggtcagagatagctctaagaagaccaccacaaacccctatgtgctgatggtgctgtacaaggacaaagtgtacaacatccagatccggtaccagaaggaaagccaggtgtacctgctgggcaccggcctgagaggcaaagaggacttcctgtctgtgtcagatatcatcgactacttcagaaagatgcctctgctgctgatcgacggcaaaaaccggggcagcagataccagtgcaccctgactcacgccgctgggtaccctcctaattccagcgccagcaacgcctctggcgccgccgctcccaccctgccggctcacccgagcacactgacccaccctcagagacggatcgatacactgaactctgacggctacacccctgagcctgcccggatcacaagcccagataagccaagacccatgcccatggacaccagcgtgtacgagagcccttattccgaccccgaggagctgaaggacaagaagctgttcctgaagagagacaacctgctgatcgccgacatcgagctgggctgcggcaacttcggctctgtgcgccaaggcgtgtaccggatgaggaagaagcagatcgatgtggccatcaaggtgcttaaacagggcacagagaaggccgacaccgaagaaatgatgcgggaggcccagatcatgcaccaactggacaatccttacatcgtgcggctgatcggcgtgtgccaggccgaggccctgatgctggtgatggaaatggccggcggaggccctctgcacaagtttctggtgggcaagcgggaagagatccctgtgtccaacgtggcggaactgctccaccaggtcagcatgggcatgaaatacctggaagagaagaacttcgtgcaccgggacctggctgccagaaatgtgctgcttgtgaacagacactacgccaagatcagcgacttcggcctgtccaaggccctgggcgccgatgatagctactacaccgccagaagcgccggaaaatggcctctgaagtggtatgcccccgagtgcatcaacttcagaaagttcagttcccggtccgacgtgtggtcctatggcgtgaccatgtgggaagccctcagctacggccagaaaccttacaagaaaatgaagggccccgaggtgatggctttcatcgagcagggaaaaagaatggaatgccctccagaatgtcctcctgaactgtacgccctcatgtccgactgctggatctacaagtgggaggatagacctgatttcctgacggtggaacagcggatgagagcctgctactactcactt

SEQ ID NO: 74 - protein, 905 aa, Artificial Sequence
  8-CAP2-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 75 - DNA, 2715 bp, Artificial Sequence
  8-CAP2-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgttcctatgatagcacatccagcgattccttatacccgagagggatccagtttaagcgacctcacacggttgcaccgtggcccccagcctaccccccagtaacgagttatcctccactgagtcagcctgacctgctccctattccacgatctcctcaacctctgggtggatcccaccgcactccaagctccaggcgcgacagcgacggcgccaactccgtggcttcttatgagaacgaggaacccgcttgtgaggacgctgacgaagacgaggatgactaccataaccccgggtacctggtggtccttcccgactcaacaccagccacgtccactgccgctccttctgcccccgcactctccacccccggcattcgggatagcgcattctctatggagtccatcgacgattatgtcaacgtccccgagtctggcgaaagcgcggaagccagcctagatggcagccgcgaatacgtcaatgtgtcacaggaactgcatcccggcgctgccaaaaccgagccagcggccttaagctcccaggaggccgaagaggtggaagaggaaggtgcaccagactacgagaatttgcaggaactaaatggaggtgggggcagccctaactctagcgcttccaatgcatctggggcagctgcaccgacgttacccgcacacccgagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 76 - protein, 708 aa, Artificial Sequence
  8-CAP4-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 77 - DNA, 2124 bp, Artificial Sequence
  8-CAP4-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaacaactacccccgcccccagaccccctacaccggcaccaactattgcctctcagcctctgtccctgcgcccagaagcttgtcggccagccgccggaggcgcagtgcatacaaggggactggatttcgcttgcgatatctatatctgggctccactggccggcacttgtggggttttgctgctgtccctggtcattacgttgtattgtggcggcgggggctcaccaaattcttcagccagtaatgcctccggggcagctgcaccaaccctcccagctcacccatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtgtgccaggccgaggctctcatgctggtcatggagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcc

SEQ ID NO: 78 - protein, 905 aa, Artificial Sequence
  8-CAP2 2Ya-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSFDSTSSDSLFPRGIQFKRPHTVAPWPPAFPPVTSFPPLSQPDLLPIPRSPQPLGGSHRTPSSRRDSDGANSVASFENEEPACEDADEDEDDFHNPGYLVVLPDSTPATSTAAPSAPALSTPGIRDSAFSMESIDDYVNVPESGESAEASLDGSREFVNVSQELHPGAAKTEPAALSSQEAEEVEEEGAPDFENLQELNGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 79 - DNA, 2715 bp, Artificial Sequence
  8-CAP2 2Ya-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgttcctttgatagcacatccagcgattccttattcccgagagggatccagtttaagcgacctcacacggttgcaccgtggcccccagccttccccccagtaacgagttttcctccactgagtcagcctgacctgctccctattccacgatctcctcaacctctgggtggatcccaccgcactccaagctccaggcgcgacagcgacggcgccaactccgtggcttcttttgagaacgaggaacccgcttgtgaggacgctgacgaagacgaggatgacttccataaccccgggtacctggtggtccttcccgactcaacaccagccacgtccactgccgctccttctgcccccgcactctccacccccggcattcgggatagcgcattctctatggagtccatcgacgattatgtcaacgtccccgagtctggcgaaagcgcggaagccagcctagatggcagccgcgaattcgtcaatgtgtcacaggaactgcatcccggcgctgccaaaaccgagccagcggccttaagctcccaggaggccgaagaggtggaagaggaaggtgcaccagacttcgagaatttgcaggaactaaatggaggtgggggcagccctaactctagcgcttccaatgcatctggggcagctgcaccgacgttacccgcacacccgagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 80 - protein, 905 aa, Artificial Sequence
  8-CAP2 2Yb-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSFDSTSSDSLFPRGIQFKRPHTVAPWPPAFPPVTSFPPLSQPDLLPIPRSPQPLGGSHRTPSSRRDSDGANSVASFENEEPACEDADEDEDDFHNPGYLVVLPDSTPATSTAAPSAPALSTPGIRDSAFSMESIDDFVNVPESGESAEASLDGSREYVNVSQELHPGAAKTEPAALSSQEAEEVEEEGAPDFENLQELNGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 81 - DNA, 2715 bp, Artificial Sequence
  8-CAP2 2Yb-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgttcctttgatagcacatccagcgattccttattcccgagagggatccagtttaagcgacctcacacggttgcaccgtggcccccagccttccccccagtaacgagttttcctccactgagtcagcctgacctgctccctattccacgatctcctcaacctctgggtggatcccaccgcactccaagctccaggcgcgacagcgacggcgccaactccgtggcttcttttgagaacgaggaacccgcttgtgaggacgctgacgaagacgaggatgacttccataaccccgggtacctggtggtccttcccgactcaacaccagccacgtccactgccgctccttctgcccccgcactctccacccccggcattcgggatagcgcattctctatggagtccatcgacgattttgtcaacgtccccgagtctggcgaaagcgcggaagccagcctagatggcagccgcgaatacgtcaatgtgtcacaggaactgcatcccggcgctgccaaaaccgagccagcggccttaagctcccaggaggccgaagaggtggaagaggaaggtgcaccagacttcgagaatttgcaggaactaaatggaggtgggggcagccctaactctagcgcttccaatgcatctggggcagctgcaccgacgttacccgcacacccgagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 82 - protein, 905 aa, Artificial Sequence
  8-CAP2 3Ya-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSFDSTSSDSLFPRGIQFKRPHTVAPWPPAFPPVTSFPPLSQPDLLPIPRSPQPLGGSHRTPSSRRDSDGANSVASFENEEPACEDADEDEDDFHNPGYLVVLPDSTPATSTAAPSAPALSTPGIRDSAFSMESIDDYVNVPESGESAEASLDGSREYVNVSQELHPGAAKTEPAALSSQEAEEVEEEGAPDFENLQELNGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 83 - DNA, 2715 bp, Artificial Sequence
  8-CAP2 3Ya-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgttcctttgatagcacatccagcgattccttattcccgagagggatccagtttaagcgacctcacacggttgcaccgtggcccccagccttccccccagtaacgagttttcctccactgagtcagcctgacctgctccctattccacgatctcctcaacctctgggtggatcccaccgcactccaagctccaggcgcgacagcgacggcgccaactccgtggcttcttttgagaacgaggaacccgcttgtgaggacgctgacgaagacgaggatgacttccataaccccgggtacctggtggtccttcccgactcaacaccagccacgtccactgccgctccttctgcccccgcactctccacccccggcattcgggatagcgcattctctatggagtccatcgacgattatgtcaacgtccccgagtctggcgaaagcgcggaagccagcctagatggcagccgcgaatacgtcaatgtgtcacaggaactgcatcccggcgctgccaaaaccgagccagcggccttaagctcccaggaggccgaagaggtggaagaggaaggtgcaccagacttcgagaatttgcaggaactaaatggaggtgggggcagccctaactctagcgcttccaatgcatctggggcagctgcaccgacgttacccgcacacccgagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 84 - protein, 905 aa, Artificial Sequence
  8-CAP2 3Yb-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSFDSTSSDSLFPRGIQFKRPHTVAPWPPAFPPVTSFPPLSQPDLLPIPRSPQPLGGSHRTPSSRRDSDGANSVASFENEEPACEDADEDEDDFHNPGYLVVLPDSTPATSTAAPSAPALSTPGIRDSAFSMESIDDYVNVPESGESAEASLDGSREFVNVSQELHPGAAKTEPAALSSQEAEEVEEEGAPDYENLQELNGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 85 - DNA, 1695 bp, Artificial Sequence
  8-CAP2 3Yb-2
acatccagcgattccttattcccgagagggatccagtttaagcgacctcacacggttgcaccgtggcccccagccttccccccagtaacgagttttcctccactgagtcagcctgacctgctccctattccacgatctcctcaacctctgggtggatcccaccgcactccaagctccaggcgcgacagcgacggcgccaactccgtggcttcttttgagaacgaggaacccgcttgtgaggacgctgacgaagacgaggatgacttccataaccccgggtacctggtggtccttcccgactcaacaccagccacgtccactgccgctccttctgcccccgcactctccacccccggcattcgggatagcgcattctctatggagtccatcgacgattatgtcaacgtccccgagtctggcgaaagcgcggaagccagcctagatggcagccgcgaattcgtcaatgtgtcacaggaactgcatcccggcgctgccaaaaccgagccagcggccttaagctcccaggaggccgaagaggtggaagaggaaggtgcaccagactacgagaatttgcaggaactaaatggaggtgggggcagccctaactctagcgcttccaatgcatctggggcagctgcaccgacgttacccgcacacccgagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 86 - protein, 905 aa, Artificial Sequence
  8-CAP2 3Yc-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCSFDSTSSDSLFPRGIQFKRPHTVAPWPPAFPPVTSFPPLSQPDLLPIPRSPQPLGGSHRTPSSRRDSDGANSVASFENEEPACEDADEDEDDFHNPGYLVVLPDSTPATSTAAPSAPALSTPGIRDSAFSMESIDDFVNVPESGESAEASLDGSREYVNVSQELHPGAAKTEPAALSSQEAEEVEEEGAPDYENLQELNGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 87 - DNA, 2715 bp, Artificial Sequence
  8-CAP2 3Yc-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgttcctttgatagcacatccagcgattccttattcccgagagggatccagtttaagcgacctcacacggttgcaccgtggcccccagccttccccccagtaacgagttttcctccactgagtcagcctgacctgctccctattccacgatctcctcaacctctgggtggatcccaccgcactccaagctccaggcgcgacagcgacggcgccaactccgtggcttcttttgagaacgaggaacccgcttgtgaggacgctgacgaagacgaggatgacttccataaccccgggtacctggtggtccttcccgactcaacaccagccacgtccactgccgctccttctgcccccgcactctccacccccggcattcgggatagcgcattctctatggagtccatcgacgattttgtcaacgtccccgagtctggcgaaagcgcggaagccagcctagatggcagccgcgaatacgtcaatgtgtcacaggaactgcatcccggcgctgccaaaaccgagccagcggccttaagctcccaggaggccgaagaggtggaagaggaaggtgcaccagactacgagaatttgcaggaactaaatggaggtgggggcagccctaactctagcgcttccaatgcatctggggcagctgcaccgacgttacccgcacacccgagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 88 - protein, 708 aa, Artificial Sequence
  8-CAP4 ZAPAS1-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVAEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 89 - DNA, 2124 bp, Artificial Sequence
  8-CAP4 ZAPAS1-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaacaactacccccgcccccagaccccctacaccggcaccaactattgcctctcagcctctgtccctgcgcccagaagcttgtcggccagccgccggaggcgcagtgcatacaaggggactggatttcgcttgcgatatctatatctgggctccactggccggcacttgtggggttttgctgctgtccctggtcattacgttgtattgtggcggcgggggctcaccaaattcttcagccagtaatgcctccggggcagctgcaccaaccctcccagctcacccatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtgtgccaggccgaggctctcatgctggtcgccgagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcc

SEQ ID NO: 90 - protein, 708 aa, Artificial Sequence
  8-CAP4 ZAPAS2-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVVQAEALMLVAEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 91 - DNA, 2124 bp, Artificial Sequence
  8-CAP4 ZAPAS2-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaacaactacccccgcccccagaccccctacaccggcaccaactattgcctctcagcctctgtccctgcgcccagaagcttgtcggccagccgccggaggcgcagtgcatacaaggggactggatttcgcttgcgatatctatatctgggctccactggccggcacttgtggggttttgctgctgtccctggtcattacgttgtattgtggcggcgggggctcaccaaattcttcagccagtaatgcctccggggcagctgcaccaaccctcccagctcacccatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtggtgcaggccgaggctctcatgctggtcgccgagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcc

SEQ ID NO: 92 - protein, 947 aa, Artificial Sequence
  8-41BB CAP2-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 93 - DNA, 2841 bp, Artificial Sequence
  8-41BB CAP2-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgtaaacggggcagaaagaaactcctgtatatattcaaacaaccatttatgagaccagtacaaactactcaagaggaagatggctgtagctgccgatttccagaagaagaagaaggaggatgtgaactgtcctatgatagcacatccagcgattccttatacccgagagggatccagtttaagcgacctcacacggttgcaccgtggcccccagcctaccccccagtaacgagttatcctccactgagtcagcctgacctgctccctattccacgatctcctcaacctctgggtggatcccaccgcactccaagctccaggcgcgacagcgacggcgccaactccgtggcttcttatgagaacgaggaacccgcttgtgaggacgctgacgaagacgaggatgactaccataaccccgggtacctggtggtccttcccgactcaacaccagccacgtccactgccgctccttctgcccccgcactctccacccccggcattcgggatagcgcattctctatggagtccatcgacgattatgtcaacgtccccgagtctggcgaaagcgcggaagccagcctagatggcagccgcgaatacgtcaatgtgtcacaggaactgcatcccggcgctgccaaaaccgagccagcggccttaagctcccaggaggccgaagaggtggaagaggaaggtgcaccagactacgagaatttgcaggaactaaatggaggtgggggcagccctaactctagcgcttccaatgcatctggggcagctgcaccgacgttacccgcacacccgagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 94 - protein, 946 aa, Artificial Sequence
  8-28 CAP2-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 95 - DNA, 2838 bp, Artificial Sequence
  8-28 CAP2-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctacgaccacacccgctccacgtcctccgactcccgcccccaccatagcttcccaacccctctccttaagacccgaggcatgccgccccgcagcgggaggcgcagtccacactaggggcctcgacttcgcttgcgatatttacatttgggctccactggccggcacctgcggtgtgttgctgctttcactggtgattacactctactgtaggagcaagaggtccaggctgctgcacagcgactacatgaatatgacccccagaaggcccggccccaccagaaagcactatcagccctacgccccccccagggactttgccgcctacaggagctcctatgatagcacatccagcgattccttatacccgagagggatccagtttaagcgacctcacacggttgcaccgtggcccccagcctaccccccagtaacgagttatcctccactgagtcagcctgacctgctccctattccacgatctcctcaacctctgggtggatcccaccgcactccaagctccaggcgcgacagcgacggcgccaactccgtggcttcttatgagaacgaggaacccgcttgtgaggacgctgacgaagacgaggatgactaccataaccccgggtacctggtggtccttcccgactcaacaccagccacgtccactgccgctccttctgcccccgcactctccacccccggcattcgggatagcgcattctctatggagtccatcgacgattatgtcaacgtccccgagtctggcgaaagcgcggaagccagcctagatggcagccgcgaatacgtcaatgtgtcacaggaactgcatcccggcgctgccaaaaccgagccagcggccttaagctcccaggaggccgaagaggtggaagaggaaggtgcaccagactacgagaatttgcaggaactaaatggaggtgggggcagccctaactctagcgcttccaatgcatctggggcagctgcaccgacgttacccgcacacccgagcaccctgactcatccacaaaggagaattgacactctgaatagcgacggatacactccggagcctgcacgaattacatcacccgacaaacctcggcctatgcccatggacacttcagtctatgagtcaccttacagcgaccccgaggaactgaaggataaaaagctctttctaaagagagataacctcttaatcgccgacatcgagctggggtgcgggaatttcggttctgttagacaaggtgtttatcggatgcgcaagaaacagatcgatgtcgctattaaggtgttgaagcagggtaccgaaaaggcggacaccgaggaaatgatgcgcgaggcccagattatgcaccagctcgacaatccctacattgtgcggctcatcggtgtgtgccaggcagaagccctcatgctggtgatggaaatggccggagggggtcctctccacaagttcctggtgggtaagcgggaagagatccctgtctccaacgtggccgagctgttacatcaggtctcaatgggaatgaagtacctcgaggaaaagaactttgtccaccgcgacctagcagccaggaatgtacttctggttaacagacactatgcaaaaattagcgacttcggccttagcaaggccctgggggcagatgactcatactatacagctagatctgccgggaagtggcctcttaagtggtatgctcccgaatgtattaactttcggaagtttagctctagaagcgatgtctggtcctacggcgtgactatgtgggaagctttatcctatggtcaaaagccttataagaaaatgaaaggccccgaggtcatggccttcatagaacagggcaaaagaatggaatgtcccccagagtgtcctccagaactctacgctctgatgtccgactgctggatatacaaatgggaggaccgaccagacttcctgaccgtagagcagcggatgagagcctgctactattctctggctagcaaagttgaaggtccccctggcagcacccagaaagctgaggcagcctgtgcc

SEQ ID NO: 96 - protein, 750 aa, Artificial Sequence
  8-41BB CAP4-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 97 - DNA, 2250 bp, Artificial Sequence
  8-41BB CAP4-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaacaactacccccgcccccagaccccctacaccggcaccaactattgcctctcagcctctgtccctgcgcccagaagcttgtcggccagccgccggaggcgcagtgcatacaaggggactggatttcgcttgcgatatctatatctgggctccactggccggcacttgtggggttttgctgctgtccctggtcattacgttgtattgtaaacggggcagaaagaaactcctgtatatattcaaacaaccatttatgagaccagtacaaactactcaagaggaagatggctgtagctgccgatttccagaagaagaagaaggaggatgtgaactgggcggcgggggctcaccaaattcttcagccagtaatgcctccggggcagctgcaccaaccctcccagctcacccatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtgtgccaggccgaggctctcatgctggtcatggagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcc

SEQ ID NO: 98 - protein, 749 aa, Artificial Sequence
  8-28 CAP4-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSGGGGSPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 99 - DNA, 2256 bp, Artificial Sequence
  8-28 CAP4-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctcttccggaacaactacccccgcccccagaccccctacaccggcaccaactattgcctctcagcctctgtccctgcgcccagaagcttgtcggccagccgccggaggcgcagtgcatacaaggggactggatttcgcttgcgatatctatatctgggctccactggccggcacttgtggggttttgctgctgtccctggtcattacgttgtattgtaggagcaagaggtccaggctgctgcacagcgactacatgaatatgacccccagaaggcccggccccaccagaaagcactatcagccctacgccccccccagggactttgccgcctacaggagcggcggcgggggctcaccaaattcttcagccagtaatgcctccggggcagctgcaccaaccctcccagctcacccatctacacttacacatcctcagagaagaatcgatactctcaactctgacggctatacgcccgaacctgcccgaattacttcacctgacaaaccaaggcccatgccaatggacacatccgtgtacgagagcccatacagtgaccctgaggagctgaaggacaagaagcttttcctgaagagggacaaccttctcatcgccgacattgaactcggatgtggtaattttgggagcgttcgccagggtgtctatcgcatgaggaagaagcagatcgatgtagcaatcaaggtgctcaaacagggtacagaaaaggctgacacggaagagatgatgcgggaggctcagatcatgcaccagctcgataatccatacatcgtgcgattgatcggcgtgtgccaggccgaggctctcatgctggtcatggagatggccgggggcggcccccttcacaagtttctggtgggaaaacgggaagaaattcctgtgagcaatgtcgccgaacttctccaccaagtctccatgggcatgaagtatctggaagagaagaactttgtgcaccgggatcttgctgcccggaacgtgctgcttgtgaatagacactatgccaaaatcagtgatttcggcctgtctaaggctctgggggcagacgattcctattataccgcacgcagtgccggtaagtggcctctgaaatggtatgcccccgagtgtattaattttcggaaattttccagccggtccgacgtgtggagctacggcgtgacaatgtgggaagccctttcttacggccaaaagccttacaagaagatgaagggacctgaggtaatggcctttatcgagcagggtaaaaggatggaatgtccgcctgagtgcccgcccgagctctacgcattgatgtccgactgttggatctacaaatgggaagatcggcctgactttctgacagtggagcagagaatgagggcatgttattattccctcgcctcaaaagtcgaaggtcccccaggaagtacacagaaagccgaagccgcatgcgcctaagtcgac

SEQ ID NO: 100 - protein, 867 aa, Artificial Sequence
  LAT-CAP2-2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNGPYSDPEELKDKKLFLKSRPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 101 - DNA, 2601 bp, Artificial Sequence
  LAT-CAP2-2
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcagcacaagcggctctggcaagcctggatctggcgagggctctaccaagggcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctgcggctgcggaagaggcaatactcgtgccctgtgtgctcgggttgttgctcctcccaatccttgcgatgttgatggctttgtgcgtacattgccacagacttcctgggagctacgattctacctcaagtgactcactgtacccaagggggatccagtttaaacgccctcacacagtggcaccctggccaccagcgtatccgccagtcacctcatacccgccgcttagtcagcccgatttgctcccgatacccaggagtcctcagcctcttggggggagccatcggactccttcctctcgaagggattccgacggcgccaattcagtagcatcctatgagaacgaggagccagcttgcgaagacgcagacgaggacgaggatgattaccacaatccggggtacctcgtagttttgcccgactccactcctgcaacgtcaaccgcggcacctagtgcgcccgcactctcaacaccgggtataagagatagcgcattttctatggagagtattgatgactacgtgaatgttcccgaatcaggtgaaagcgctgaggcttcattggatgggtcacgggaatatgtgaacgtttctcaagaactccacccgggtgccgcaaaaacggagcccgcggcattgagcagccaggaagctgaagaggtggaagaggaaggcgcgccggactatgaaaatctgcaggagctcaatggcccttactctgacccggaagaacttaaggataaaaagctgttcctgaaatcaaggccaaatagctcagcctcaaatgcgagcggcgcagctgcaccgactcttccagctcacccatccaccttgacgcacccccagcgacgaatagatactctcaattcagatggctatacgccggaacccgctcgcatcacgtctccagataagcctcgacccatgcccatggacacctcagtgtacgaaagcccctactccgatccggaggaacttaaggacaaaaaactgtttttgaaaagagataatctgttgattgcggacattgaactcgggtgcggtaatttcggaagcgtcaggcaaggcgtttataggatgaggaaaaagcagatcgatgtcgctattaaggtattgaagcaagggacggagaaggcggacacggaggagatgatgcgagaggctcaaatcatgcatcaactcgacaacccctacattgttagattgattggagtttgtcaagcagaagccttgatgcttgtcatggagatggccgggggcggacccctccacaagtttttggtggggaagcgcgaagaaatcccggtgtctaacgtagcagagcttttgcatcaggttagcatgggtatgaagtatttggaggagaagaacttcgtacatagagatcttgcagctcgcaatgttctcctcgtaaacaggcactacgcaaaaataagtgattttggcctgagtaaggcgctcggcgccgatgattcttactacactgctagaagcgctgggaaatggcccttgaaatggtatgcacctgaatgcattaacttccgaaaatttagttcccggtccgatgtttggtcttacggcgtgactatgtgggaagctctctcctacggccaaaaaccctacaaaaaaatgaagggacctgaggtgatggcgttcattgaacagggtaaacggatggaatgcccacccgaatgcccgcctgaattgtacgcgcttatgtccgactgttggatctacaagtgggaagatcgaccggactttctcaccgtcgaacaacggatgagagcttgttactactctctt